(19) United States
(12) Patent Application Publication
(10) Pub. No.: US 2022/0089704 A1
(43) Pub. Date: Mar. 24, 2022

(54) HUMANIZED AND STABILIZED FC5 VARIANTS FOR ENHANCEMENT OF BLOOD BRAIN BARRIER TRANSPORT

(71) Applicants: Biogen MA Inc., Cambridge, MA (US); National Research Council of Canada, Ottawa (CA)

(72) Inventors: Benjamin A. Smith, Belmont, MA (US); Andreas Lehmann, Belmont, MA (US); Thomas O. Cameron, Cambridge, MA (US); R. Blake Pepinsky, Arlington, MA (US); Dingyi Wen, Waltham, MA (US); Graham K. Farrington, Acton, MA (US); Gopalan Raghunathan, San Francisco, CA (US); Nels E. Pederson, Mansfield, MA (US); Danica Stanimirovic, Ottawa (CA); Traian Sulea, Kirkland (CA); Arsalan S. Haqqani, Kanata (CA)

(21) Appl. No.: 17/288,445

(22) PCT Filed: Oct. 28, 2019
(86) PCT No.: PCT/US19/58286

§ 371 Date: Apr. 23, 2021

Related U.S. Application Data
(60) Provisional application No. 62/751,962, filed on Oct. 29, 2018.

(51) Int. Cl. C07K 16/18; A61P 25/28; C07K 16/28; C07K 14/47

(52) U.S. Cl. CPC C07K 16/18; A61P 25/28; A61K 2039/505; C07K 14/4711; C07K 16/2803

(57) ABSTRACT

Featured are compositions comprising humanized and engineered variants of an antibody variable domain (e.g., FC5), chimeric molecules comprising same, compositions comprising same, and uses thereof.

### CROSS-REFERENCE TO RELATED APPLICATIONS

This application is a U.S. National Stage of International Patent Application No. PCT/US2019/058286, filed Oct. 28, 2019, which claims the benefit of priority of U.S. Provisional Application No. 62/751,962, filed Oct. 29, 2018, the contents of which are incorporated by reference herein in their entireties.

### TECHNICAL FIELD

This disclosure relates generally to compositions for delivering a desired agent (e.g., a therapeutic, a diagnostic) across the blood brain barrier and methods of using same for treatment and/or prevention of neurological disorders.

### BACKGROUND

The delivery of drugs to the central nervous system (CNS) has been a challenge in the treatment of neurological diseases such as Alzheimer's disease and Parkinson's disease. For drugs to reach the nervous system, they first have to penetrate the blood brain barrier (BBB), which is a major challenge due to the selectivity of the BBB. The BBB acts as a semipermeable membrane, preventing most molecules from entering the nervous system from the blood and allows only low molecular weight (<400 Da) and lipophilic compounds to pass. Most small molecules and large molecules, such as monoclonal antibodies and antisense oligonucleotides, cannot pass through this barrier. Due to this challenging process of drug penetration across the BBB, a small fraction of therapeutic agents for neurological diseases make it to clinical trials.

There is a need in the art for improved compositions and methods for delivering a therapeutic agent to the CNS.

### SUMMARY

This application relates to molecules comprising an antibody variable domain (e.g., an FC5 variant) that can be used to transport a therapeutic agent across the blood brain barrier.

In one aspect, the disclosure features an antibody variable domain that transmigrates across the blood brain barrier. The antibody variable domain comprises an amino acid sequence that is at least 85% identical to the sequence set forth in SEQ ID NO:1. The amino acid sequence comprises: (i) amino acid substitutions, as compared to SEQ ID NO:1, at one or more of the positions corresponding to positions 5, 6, 14, 75, 87, 88, 93, 114, and 117 of SEQ ID NO:1; and (ii) complementarity determining region (CDR)1 comprising the sequence set forth in SEQ ID NO:47 or 57, CDR2 comprising the sequence set forth in SEQ ID NO:50, and CDR3 comprising the sequence set forth in SEQ ID NO:51.

In some instances, the amino acid sequence comprises amino acid substitutions, as compared to SEQ ID NO:1, at at least four of the positions corresponding to positions 5, 6, 14, 75, 87, 88, 93, 114, and 117 of SEQ ID NO:1. In other instances, the amino acid sequence comprises amino acid substitutions, as compared to SEQ ID NO:1, at at least five of the positions corresponding to positions 5, 6, 14, 75, 87, 88, 93, 114, and 117 of SEQ ID NO:1. In yet other instances, the amino acid sequence comprises amino acid substitutions, as compared to SEQ ID NO:1, at at least six of the positions corresponding to positions 5, 6, 14, 75, 87, 88, 93, 114, and 117 of SEQ ID NO:1. In one instance, the amino acid sequence comprises amino acid substitutions, as compared to SEQ ID NO:1, at each of the positions corresponding to positions 5, 6, 14, 75, 87, 88, 93, 114, and 117 of SEQ ID NO:1.

In certain instances, CDR1 comprises the sequence set forth in SEQ ID NO:47. In other instances, CDR1 comprises the sequence set forth in SEQ ID NO:57.

In some cases, the amino acid sequence comprises one or more (i.e., 1, 2, 3, 4, 5, 6, 7, 8, or 9) of: valine at the position corresponding to position 5 of SEQ ID NO:1; glutamic acid at the position corresponding to position 6 of SEQ ID NO:1; proline at the position corresponding to position 14 of SEQ ID NO:1; serine at the position corresponding to position 75 of SEQ ID NO:1; arginine at the position corresponding to position 87 of SEQ ID NO:1; alanine at the position corresponding to position 88 of SEQ ID NO:1; valine at the position corresponding to position 93 of SEQ ID NO:1; glutamine at the position corresponding to position 114 of SEQ ID NO:1; or leucine at the position corresponding to position 117 of SEQ ID NO:1.

In certain cases, the amino acid sequence comprises: valine at the position corresponding to position 5 of SEQ ID NO:1; glutamic acid at the position corresponding to position 6 of SEQ ID NO:1; proline at the position corresponding to position 14 of SEQ ID NO:1; serine at the position corresponding to position 75 of SEQ ID NO:1; arginine at the position corresponding to position 87 of SEQ ID NO:1; alanine at the position corresponding to position 88 of SEQ ID NO:1; valine at the position corresponding to position 93 of SEQ ID NO:1; glutamine at the position corresponding to position 114 of SEQ ID NO:1; and leucine at the position corresponding to position 117 of SEQ ID NO:1.

In some instances, the amino acid sequence comprises substitutions, as compared to SEQ ID NO:1, at one or more (i.e., 1, 2, 3, 4, 5, or 6) of the positions corresponding to positions 1, 37, 44, 45, 47, or 79 of SEQ ID NO:1. In some embodiments, the amino acid sequence comprises one or more (i.e., 1, 2, 3, 4, 5, or 6) of: glutamic acid at the position corresponding to position 1 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; or leucine at the position corresponding to position 79 of SEQ ID NO:1. In some cases, the amino acid sequence comprises, as compared to SEQ ID NO:1, substitutions at the positions corresponding to positions 44 and 45 of SEQ ID NO:1. In certain embodiments, the amino acid sequence comprises: glycine at the position corresponding to position 44 of SEQ ID NO:1; and leucine at the position corresponding to position 45 of SEQ ID NO:1. In other cases, the amino acid sequence comprises, as compared to SEQ ID NO:1, substitutions at the positions corresponding to positions 1, 44 and 45 of SEQ ID NO:1. In certain embodiments, the amino acid sequence comprises: glutamic acid at the position corresponding to position 1 of SEQ ID NO:1; glycine at the position corresponding to position 44 of SEQ ID NO:1; and leucine at the position corresponding to position 45 of SEQ ID NO:1. In yet other cases, the amino acid sequence comprises, as compared to SEQ ID NO:1, substitutions at the positions corresponding to positions 44, 45, and 47 of SEQ ID NO:1. In some embodiments, the amino acid sequence comprises: glycine at the position corresponding to position 44 of SEQ ID NO:1; leucine at the position corresponding to position 45 of SEQ ID NO:1; and tryptophan at the position corresponding to position 47 of SEQ ID NO:1. In some cases, the amino acid sequence comprises, as compared to SEQ ID NO:1, substitutions at the positions corresponding to positions 1, 44, 45, and 47 of SEQ ID NO:1. In certain embodiments, the amino acid sequence comprises: glutamic acid at the position corresponding to position 1 of SEQ ID NO:1; glycine at the position corresponding to position 44 of SEQ ID NO:1; leucine at the position corresponding to position 45 of SEQ ID NO:1; and tryptophan at the position corresponding to position 47 of SEQ ID NO:1. In certain cases, the amino acid sequence comprises, as compared to SEQ ID NO:1, substitutions at the positions corresponding to positions 37, 44, 45, and 47 of SEQ ID NO:1. In some embodiments, the amino acid sequence comprises: valine at the position corresponding to position 37 of SEQ ID NO:1; glycine at the position corresponding to position 44 of SEQ ID NO:1; leucine at the position corresponding to position 45 of SEQ ID NO:1; and tryptophan at the position corresponding to position 47 of SEQ ID NO:1. In other instances, the amino acid sequence comprises, as compared to SEQ ID NO:1, substitutions at the positions corresponding to positions 1, 37, 44, 45, and 47 of SEQ ID NO:1. In certain embodiments, the amino acid sequence comprises: glutamic acid at the position corresponding to position 1 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; and tryptophan at the position corresponding to position 47 of SEQ ID NO:1. In some instances, the amino acid sequence comprises, as compared to SEQ ID NO:1, substitutions at the positions corresponding to positions 37, 44, 45, 47, and 79 of SEQ ID NO:1. In certain embodiments, the amino acid sequence comprises: valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; and leucine at the position corresponding to position 79 of SEQ ID NO:1.

In certain instances, the amino acid sequence is the sequence set forth in any one of SEQ ID NO:10 to 17.

In another aspect, the disclosure relates to an antibody variable domain that transmigrates across the blood brain barrier, wherein the antibody variable domain comprises an amino acid sequence that is at least 85% identical to SEQ ID NO:1. The amino acid sequence comprises complementarity determining region (CDR)1, CDR2, and CDR3, wherein CDR2 comprises the sequence set forth in SEQ ID NO:50. In one embodiment, CDR1 comprises the sequence set forth in SEQ ID NO:47, CDR3 comprises the sequence set forth in SEQ ID NO:51, and the amino acid sequence comprises cysteines at the positions corresponding to positions 49 and 70 of SEQ ID NO:1. In another embodiment, CDR1 comprises the sequence set forth in SEQ ID NO:48, CDR3 comprises the sequence set forth in SEQ ID NO:51, and the amino acid sequence comprises cysteine at the position corresponding to position 79 of SEQ ID NO:1. In yet another embodiment, CDR1 comprises the sequence set forth in SEQ ID NO:49, and CDR3 comprises the sequence set forth in SEQ ID NO:52. In another embodiment, CDR1 comprises the sequence set forth in SEQ ID NO:58, and CDR3 comprises the sequence set forth in SEQ ID NO:52. In another embodiment, CDR1 comprises the sequence set forth in SEQ ID NO:49, and CDR3 comprises the sequence set forth in SEQ ID NO:53. In another embodiment, CDR1 comprises the sequence set forth in SEQ ID NO:58, and CDR3 comprises the sequence set forth in SEQ ID NO:53. In yet another embodiment, CDR1 comprises the sequence set forth in SEQ ID NO:49, and CDR3 comprises the sequence set forth in SEQ ID NO:54. In another embodiment, CDR1 comprises the sequence set forth in SEQ ID NO:58, and CDR3 comprises the sequence set forth in SEQ ID NO:54. In another embodiment, CDR1 comprises the sequence set forth in SEQ ID NO:49, and CDR3 comprises the sequence set forth in SEQ ID NO:55. In yet another embodiment, CDR1 comprises the sequence set forth in SEQ ID NO:58, and CDR3 comprises the sequence set forth in SEQ ID NO:55.

In some instances, the amino acid sequence comprises, as compared to SEQ ID NO:1, substitutions at one or more (e.g., 1, 2, 3, 4, 5, 6, 7, 8, 9, 10, 11, 12, 13, or 14) of the positions corresponding to positions 1, 5, 6, 14, 37, 44, 45, 47, 75, 87, 88, 93, 114, or 117 of SEQ ID NO:1. In some embodiments, the amino acid sequence comprises one or more (e.g., 1, 2, 3, 4, 5, 6, 7, 8, 9, 10, 11, 12, 13, or 14) of: glutamic acid at the position corresponding to position 1 of SEQ ID NO:1; valine at the position corresponding to position 5 of SEQ ID NO:1; glutamic acid at the position corresponding to position 6 of SEQ ID NO:1; proline at the position corresponding to position 14 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; serine at the position corresponding to position 75 of SEQ ID NO:1; arginine at the position corresponding to position 87 of SEQ ID NO:1; alanine at the position corresponding to position 88 of SEQ ID NO:1; valine at the position corresponding to position 93 of SEQ ID NO:1; glutamine at the position corresponding to position 114 of SEQ ID NO:1; or leucine at the position corresponding to position 117 of SEQ ID NO:1.

In certain instances, the amino acid sequence comprises, as compared to SEQ ID NO:1, substitutions at one or more (e.g., 1, 2, 3, 4, 5, 6, 7, 8, or 9) of the positions corresponding to positions 5, 6, 14, 75, 87, 88, 93, 114, or 117 of SEQ ID NO:1. In one embodiment, the amino acid sequence comprises one or more (e.g., 1, 2, 3, 4, 5, 6, 7, 8, or 9) of: valine at the position corresponding to position 5 of SEQ ID NO:1; glutamic acid at the position corresponding to position 6 of SEQ ID NO:1; proline at the position corresponding to position 14 of SEQ ID NO:1; serine at the position corresponding to position 75 of SEQ ID NO:1; arginine at the position corresponding to position 87 of SEQ ID NO:1; alanine at the position corresponding to position 88 of SEQ ID NO:1; valine at the position corresponding to position 93 of SEQ ID NO:1; glutamine at the position corresponding to position 114 of SEQ ID NO:1; or leucine at the position corresponding to position 117 of SEQ ID NO:1.

In other instances, the amino acid sequence comprises substitutions, as compared to SEQ ID NO:1, at the positions corresponding to positions 44 and 45 of SEQ ID NO:1. In some embodiments, the amino acid sequence comprises: glycine at the position corresponding to position 44 of SEQ ID NO:1; and leucine at the position corresponding to position 45 of SEQ ID NO:1.

In yet another instance, the amino acid sequence comprises substitutions, as compared to SEQ ID NO:1, at the positions corresponding to positions 1, 44 and 45 of SEQ ID NO:1. In one embodiment, the amino acid sequence comprises: glutamic acid at the position corresponding to position 1 of SEQ ID NO:1; glycine at the position corresponding to position 44 of SEQ ID NO:1; and leucine at the position corresponding to position 45 of SEQ ID NO:1.

In some instances, the amino acid sequence comprises substitutions, as compared to SEQ ID NO:1, at the positions corresponding to positions 44, 45, and 47 of SEQ ID NO:1. In one embodiment, the amino acid sequence comprises: glycine at the position corresponding to position 44 of SEQ ID NO:1; leucine at the position corresponding to position 45 of SEQ ID NO:1; and tryptophan at the position corresponding to position 47 of SEQ ID NO:1.

In another instance, the amino acid sequence comprises substitutions, as compared to SEQ ID NO:1, at the positions corresponding to positions 1, 44, 45, and 47 of SEQ ID NO:1. In one embodiment, the amino acid sequence comprises: glutamic acid at the position corresponding to position 1 of SEQ ID NO:1; glycine at the position corresponding to position 44 of SEQ ID NO:1; leucine at the position corresponding to position 45 of SEQ ID NO:1; and tryptophan at the position corresponding to position 47 of SEQ ID NO:1.

In certain instances, the amino acid sequence comprises substitutions, as compared to SEQ ID NO:1, at the positions corresponding to positions 37, 44, 45, and 47 of SEQ ID NO:1. In one embodiment, the amino acid sequence comprises: valine at the position corresponding to position 37 of SEQ ID NO:1; glycine at the position corresponding to position 44 of SEQ ID NO:1; leucine at the position corresponding to position 45 of SEQ ID NO:1; and tryptophan at the position corresponding to position 47 of SEQ ID NO:1.

In some instances, the amino acid sequence comprises substitutions, as compared to SEQ ID NO:1, at the positions corresponding to positions 1, 37, 44, 45, and 47 of SEQ ID NO:1. In one embodiment, the amino acid sequence comprises: glutamic acid at the position corresponding to position 1 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; and tryptophan at the position corresponding to position 47 of SEQ ID NO:1.

In another instance, the amino acid sequence comprises substitutions, as compared to SEQ ID NO:1, at the positions corresponding to positions 37, 44, 45, 47, and 79 of SEQ ID NO:1. In one embodiment, the amino acid sequence comprises: valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; and leucine at the position corresponding to position 79 of SEQ ID NO:1.

In certain instances, the amino acid sequence comprises substitutions, as compared to SEQ ID NO:1, at the positions corresponding to positions 1, 37, 44, 45, 47, and 79 of SEQ ID NO:1. In one embodiment, the amino acid sequence comprises: glutamic acid at the position corresponding to position 1 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; and leucine at the position corresponding to position 79 of SEQ ID NO:1.

In one embodiment, the amino acid sequence is the sequence set forth in SEQ ID NO: 20. In another embodiment, the amino acid sequence is the sequence set forth in SEQ ID NO: 21. In yet another embodiment, the amino acid sequence is the sequence set forth in SEQ ID NO: 22. In another embodiment, the amino acid sequence is the sequence set forth in SEQ ID NO: 23. In another embodiment, the amino acid sequence is the sequence set forth in SEQ ID NO: 24. In another embodiment, the amino acid sequence is the sequence set forth in SEQ ID NO: 25. In another embodiment, the amino acid sequence is the sequence set forth in SEQ ID NO: 26. In another embodiment, the amino acid sequence is the sequence set forth in SEQ ID NO: 27. In yet another embodiment, the amino acid sequence is the sequence set forth in SEQ ID NO:28.

In another aspect, the disclosure features a chimeric molecule comprising any of the antibody variable domains described above.

In certain instances, the chimeric molecule comprises an antibody Fc region. In some instances, the chimeric molecule comprises an antibody, an antigen-binding fragment of an antibody (e.g., a Fab), a single chain antibody, a diabody, a nanobody, an enzyme, a nucleic acid (e.g., an antisense oligonucleotide), a small molecule drug, or a liposome or a lipid nanoparticle encapsulating a nucleic acid (e.g., an antisense oligonucleotide), a small molecule drug, or a peptide. In some instances, the chimeric molecule comprises a display scaffold, such as ankyrin-repeats (Darpins), fibronectin (Adnectins), protein A (Affibodies), or Stefin A (Affimers).

In some instances, in the chimeric molecule, the antibody variable domain is linked directly or via an intervening amino acid sequence to the N-terminus of a hinge region of an antibody. The C-terminus of the hinge region is fused to an Fc moiety. In certain embodiments, the chimeric molecule is bivalent with respect to the antibody variable domain (i.e., it comprises two antibody variable domains, each linked directly or via an intervening amino acid sequence to the N-terminus of a hinge region of an antibody, and wherein the C-terminus of the hinge region is fused to an Fc moiety).

In certain cases, the hinge region comprises the sequence AEPKSCD (SEQ ID NO:56), AEPKSSD (SEQ ID NO:59), or KTHTCPPCP (SEQ ID NO:19).

In some cases, the intervening amino acid sequence comprises: a heavy chain variable region of an antibody; a light chain variable region of an antibody; an enzyme; a peptide, or a linker comprising the amino acid sequence 3X(G4S) (SEQ ID NO:60), G4S (SEQ ID NO:5), GG, or G.

In some cases, the Fc moiety is a hIgG1 Fc, a hIgG2 Fc, a hIgG3 Fc, a hIgG4 Fc, a hIgG1agly Fc, a hIgG2 SAA Fc, a hIgG4(S228P) Fc, a hIgG4(S228P)/G1 agly Fc (in this format—that minimizes effector function—the CH1 and CH2 domains are IgG4 with a ‘fixed’ hinge (S228P) and is aglycosylated. The CH3 domain is hIgG1, or a hIgG4(S228P) agly Fc.

In certain cases, the Fc moiety is linked directly or via a second intervening amino acid sequence to a polypeptide comprising a second antibody variable domain (e.g., the variable domain(s) of a therapeutic antibody); an Fab; an scFv; a single domain antibody; a nanobody, a diabody, a peptide; an enzyme; or to a nucleic acid (e.g., an antisense oligonucleotide).

In some cases, in the chimeric molecule, the hinge region is linked to a nucleic acid. In some cases, in the chimeric molecule, the hinge region is chemically conjugated to a nucleic acid. In certain embodiments, the nucleic acid is an antisense oligonucleotide.

In another aspect, the disclosure provides a chimeric molecule comprising an antibody variable domain described herein linked directly or via an intervening amino acid sequence to a liposome, polymeric nanocarrier, or lipid nanoparticle.

In some instances, the liposome, polymeric nanocarrier, or lipid nanoparticle encapsulates a small molecule or a nucleic acid. In some instances the liposome, polymeric nanocarrier, or lipid nanoparticle encapsulates a peptide. In some instances the liposome, polymeric nanocarrier, or lipid nanoparticle encapsulates an enzyme. In some instances the liposome, polymeric nanocarrier, or lipid nanoparticle encapsulates an antibody or antibody fragment (including Fab, Fab′2, scFv). In some instances the liposome, polymeric nanocarrier, or lipid nanoparticle encapsulates a nanobody. In some instances the liposome, polymeric nanocarrier, or lipid nanoparticle encapsulates an antisense oligonucleotide. In some embodiments, the antisense oligonucleotide is a gapmer. In other embodiments, the antisense oligonucleotide is a splice switching antisense oligonucleotide.

In another aspect, the disclosure provides a chimeric molecule comprising an antibody variable domain described herein, wherein the antibody variable domain is linked directly or via an intervening amino acid sequence to a whole antibody. In some embodiments, the whole antibody is an antibody that binds to human LINGO-1, human LINGO-2, human LINGO-3, human LINGO-4, human NOGO receptor human TREM2, a dipeptide repeat (DPR) of poly-glycine-alanine (GA) having at least 6 repeats (GA)₆ as translated from the human chromosome 9 open reading frame 72 (C9orf72) gene (a human C9orf72 DPR), human TWEAK, human TWEAK receptor, human beta amyloid, human tau, human alpha-synuclein, human TDP-43, human DR6, human SOD1, DR6, JC virus (or other infectious disease targets), or human ErbB2, VEGF, CD20, Her2 (or other human brain cancer targets). In some instances, the whole antibody is bevacizumab, rituximab, trastuzumab, an anti-PD1 antibody, or an anti-PDL-L1 antibody.

In another aspect, the disclosure provides a chimeric molecule comprising an antibody variable domain described herein and a human serum albumin, or human serum albumin binding moiety. In some instances, this chimeric molecule further comprises a therapeutic agent. In one embodiment, the therapeutic agent is an antibody variable domain (e.g., the variable domain(s) of a therapeutic antibody); an Fab; an scFv; a single domain antibody; a nanobody, a diabody, a peptide; an enzyme; a nucleic acid (e.g., an antisense oligonucleotide).

In another aspect, the disclosure provides a chimeric molecule that encompasses any of the molecules shown in FIG. 49. In some cases, the Fc region of the illustrated molecules is replaced by a human serum albumin and these chimeric molecules are also part of this disclosure. In some instances, the cargos is linked using chemical conjugation such as sulfhydryl (maleimide, iodo, vinyl sulfone).

In yet another aspect, the disclosure provides a chimeric molecule comprising an antibody variable domain described herein linked directly or via an intervening amino acid sequence to a viral capsid (e.g., AAV) containing a therapeutic vector. See. e.g., Naso et al., BioDrugs. 2017; 31(4): 317-334.

In another aspect the disclosure features a composition comprising: (1) a chimeric protein that comprises (i) an antibody variable domain described herein, and (ii) a first protein comprising a first hinge region of an antibody and a first Fc moiety, wherein the C-terminus of the first hinge region is linked to the N-terminus of the first Fc moiety, and wherein the C-terminus of the antibody variable domain is fused directly or via an intervening amino acid sequence to the N-terminus of the first hinge region; (2) a second protein comprising a second hinge region of an antibody and a second Fc moiety, wherein the C-terminus of the second hinge region is linked to the N-terminus of the second Fc moiety, wherein the second protein pairs with the first protein; and (3) a therapeutic agent. In some instances, a linker connects the first antibody variable to the first protein; and/or the chimeric protein to the therapeutic agent. In some cases, the linker is 3X(G4S) (SEQ ID NO:60), G4S (SEQ ID NO:5), GG, or G.

In another aspect, the disclosure provides a composition comprising: (1) a first chimeric protein that comprises (i) a first antibody variable domain of any one of the antibody variable domains disclosed herein; and (ii) a first protein comprising a first hinge region of an antibody and a first Fc moiety, wherein the C-terminus of the first hinge region is linked to the N-terminus of the first Fc moiety, and wherein the C-terminal of the first antibody variable domain is fused directly or via an intervening amino acid sequence to the N-terminus of the first hinge region; (2) a second chimeric protein that comprises (i) a second antibody variable domain of any one of the antibody variable domains disclosed herein; and (ii) a second protein comprising a second hinge region of an antibody and a second Fc moiety, wherein the C-terminus of the second hinge region is linked to the N-terminus of the second Fc moiety, wherein the C-terminal of the second antibody variable domain is fused directly or via an intervening amino acid sequence to the N-terminus of the second hinge region, and wherein the second protein pairs with the first protein; and (3) a therapeutic agent. In some instances, a linker connects the first antibody variable to the first protein; and/or the second antibody variable to the second protein; and/or the chimeric protein to the therapeutic agent. In some cases, the linker is 3X(G4S) (SEQ ID NO:60), G4S (SEQ ID NO:5), GG, or G.

In another aspect, the disclosure features a composition comprising: (1) a first chimeric protein that comprises (i) a first antibody variable domain of any one of the antibody variable domains disclosed herein; and (ii) a first protein comprising a first hinge region of an antibody and a first Fc moiety, wherein the C-terminus of the first hinge region is linked to the N-terminus of the first Fc moiety, and wherein the C-terminal of the first antibody variable domain is fused via a first intervening amino acid sequence to the N-terminus of the first hinge region; and (2) a second chimeric protein that comprises (i) a second antibody variable domain any one of the antibody variable domains disclosed herein; and (ii) a second protein comprising a second hinge region of an antibody and a second Fc moiety, wherein the C-terminus of the second hinge region is linked to the N-terminus of the second Fc moiety, wherein the C-terminal of the second antibody variable domain is fused via a second intervening amino acid sequence to the N-terminus of the second hinge region, and wherein the second protein pairs with the first protein; wherein the first and second intervening amino acid sequence comprise Fabs that are therapeutic agents. In some instances, a linker connects the first antibody variable to the first protein; and/or the second antibody variable to the second protein; and/or the chimeric protein to the therapeutic agent. In some cases, the linker is 3X(G4S) (SEQ ID NO:60), G4S (SEQ ID NO:5), GG, or G.

In some instances, the hinge region comprises the sequence AEPKSCD (SEQ ID NO:56) or AEPKSSD (SEQ ID NO:59). In other instances, the hinge region comprises the sequence KTHTCPPCP (SEQ ID NO:19).

In certain instances, the first and second Fc moieties have an identical amino acid sequence. In other instances, the first and second Fc moieties have different amino acid sequences.

In some instances, the first and/or second Fc moieties are aglycosylated.

In certain instances, the first and second Fc moieties are selected from the group consisting of hIgG1 Fc, hIgG2 Fc, hIgG3 Fc, hIgG4 Fc, hIgG1agly Fc, h IgG2 SAA Fc, hIgG4(S228P) Fc, hIgG4(S228P)/G1 agly Fc, and hIgG4(S228P) agly Fc.

In some instances, the compositions described above further comprise a moiety that extends half-life. In some cases, the first and/or second Fc moieties are mutated variants of the wild type Fc that serve to extend half-life. Fc variants, such as YTE and LS, have resulted in enhanced antibody half-life in humans. In some instances, the variant Fc region differs from a parent Fc region in that the variant Fc region comprises amino acid substitutions at positions 428 and 434, wherein said amino acid substitutions are M428L and N434S. See, e.g., U.S. Pat. Nos. 8,546,543 and 8,624,007; US20140056879A1; Brian J. Booth, et al. (2018) Extending human IgG half-life using structure-guided design, mAbs, DOI: 10.1080/19420862.2018.1490119; and Shen Y, et al. (2017) Increased half-life and enhanced potency of Fc-modified human PCSK9 monoclonal antibodies in primates. PLoS ONE 12(8): e0183326. doi.org/10.1371/journal.pone.0183326. In other cases, the composition further comprises a HSA mutant that extends half-life. See. e.g., U.S. Pat. No. 8,748,380; and WO2014179657A1. In some instances, the HSA variant is mutated at one or more residues selected from the group consisting of Y411, V415, V418, T422, L423, V426, L430, L453, L457, L460, V473, R485, F488, L491, F502, F507, F509, K525, A528, L529, L532, V547, M548, F551, K573, L575, V576, 5579, and L583. In yet other cases, the composition further comprises a XTEN. See, e.g., WO2013130683A2 and Podust et al., Journal of Controlled Release, Volume 240, 28 Oct. 2016, Pages 52-66.

In some instances, the first and second hinge regions have an identical amino acid sequence.

In certain instances, the therapeutic agent is a binding molecule that comprises an antibody variable domain. In one embodiment, the binding molecule binds to human LINGO-1, human LINGO-2, human LINGO-3, human LINGO-4, human TREM2, a human C9orf72 DPR), human TWEAK, human TWEAK receptor, human beta amyloid, human tau, human alpha-synuclein, or human TDP-43. In some cases, the binding molecule is an Fab and the N-terminus of the VH or VL domain of the Fab is linked to the C-terminus of the first Fc moiety.

In some instances, the therapeutic agent is an antisense oligonucleotide. In one embodiment, the antisense oligonucleotide is a gapmer. In other embodiments, the antisense oligonucleotide is a splice switching antisense oligonucleotide. In some cases, the antisense oligonucleotide is linked to the first and/or second hinge region.

In certain instances, the therapeutic agent is a small molecule drug, peptide, enzyme, nanobody, or a nucleic acid (e.g., antisense oligonucleotide) encapsulated in a liposome, polymeric nanocarrier, or lipid nanoparticle.

In another aspect, the disclosure provides a composition comprising: (1) a chimeric protein comprising (i) the antibody variable domain of any one of the antibody variable domains described herein, and (ii) a light chain of an antibody, wherein the C-terminus of the antibody variable domain is fused directly or via an intervening amino acid sequence to the N-terminus of the light chain of the antibody; and (2) a heavy chain of the antibody that pairs with the light chain of the antibody.

In another aspect, the disclosure features a composition comprising: (1) a chimeric protein comprising (i) the antibody variable domain of any one of the antibody variable domains described herein; and (ii) a heavy chain of an antibody, wherein the C-terminus of the antibody variable domain is fused directly or via an intervening amino acid sequence to the N-terminus of the heavy chain of the antibody; and (2) a light chain of the antibody that pairs with the heavy chain of the antibody.

In another aspect, the disclosure provides a composition comprising: (1) a first chimeric protein comprising (i) a first antibody variable domain of any one of the antibody variable domains described herein, and (ii) a light chain of an antibody, wherein the C-terminus of the first antibody variable domain is fused directly or via an intervening amino acid sequence to the N-terminus of the light chain of the antibody; and (2) a second chimeric protein comprising (i) a second antibody variable domain of any one of the antibody variable domains described herein, and (ii) a heavy chain of the antibody, wherein the C-terminus of the second antibody variable domain is fused directly or via an intervening amino acid sequence to the N-terminus of the heavy chain of the antibody.

In some instances, the intervening amino acid sequence is 3X(G4S) (SEQ ID NO:60), G4S (SEQ ID NO:5), GG, or G.

In certain instances, the antibody binds to human LINGO-1, human LINGO-2, human LINGO-3, human LINGO-4, human TREM2, human C9orf72 DPR, human TWEAK, human TWEAK receptor, human beta amyloid, human tau, human alpha-synuclein, or human TDP-43. In some embodiments, the antibody comprises the six CDRs of an antibody selected from the group consisting of Li81, Li113, huP2D10, 12F6A, 12F4, 21D11, 6C5, 40E8, BMS-986168, 1507, 5J10, 41D1, 14W3, and 51C1. In other embodiments, the antibody comprises the heavy chain variable region (VH) and light chain variable region (VL) of an antibody selected from the group consisting of Li81, Li113, huP2D10, 12F6A, 12F4, 21D11, 6C5, 40E8, BMS-986168, 1507, 5J10, 41D1, 14W3, and 51C1. In yet other embodiments, the antibody comprises the heavy chain and light chain of an antibody selected from the group consisting of Li81, Li113, huP2D10, 12F6A, 12F4, 21D11, 6C5, 40E8, BMS-986168, 1507, 5J10, 41D1, 14W3, and 51C1.

In another aspect, the disclosure features a composition comprising a chimeric protein comprising (i) the antibody variable domain of any one of the antibody variable domains described herein, (ii) a protein comprising a hinge region of an antibody and an Fc moiety, wherein the C-terminus of the antibody variable domain is fused directly or via an intervening amino acid sequence to the N-terminus of the hinge region of the protein, and (iii) a therapeutic agent. In some cases, the composition comprises a dimer of the chimeric protein. In some cases, the hinge region comprises the sequence AEPKSCD (SEQ ID NO:56) or AEPKSSD (SEQ ID NO:59). In other cases, the hinge region comprises the sequence KTHTCPPCP (SEQ ID NO:19). In certain cases, the Fc moiety is aglycosylated. In some cases, the Fc moiety is selected from the group consisting of hIgG1 Fc, hIgG2 Fc, hIgG3 Fc, hIgG4 Fc, hIgG1agly Fc, h IgG2 SAA Fc, hIgG4(S228P)/G1 agly Fc, hIgG4(S228P) Fc, and hIgG4(S228P) agly Fc. In some cases, the C-terminus of the antibody variable domain is fused directly to the N-terminus of the hinge region of the protein. In certain cases, the therapeutic agent is a binding molecule that comprises a second antibody variable domain. In some embodiments, the binding molecule binds to human LINGO-1, human LINGO-2, human LINGO-3, human LINGO-4, human TREM2, human C9orf72 DPRs, human TWEAK, human TWEAK receptor, human beta amyloid, human tau, human alpha-synuclein, or human TDP-43. In some instances, the binding molecule is an Fab and the N-terminus of the VH or VL domain of the Fab is linked to the C-terminus of the Fc moiety. In certain instances, the therapeutic agent is an antisense oligonucleotide. In one embodiment, the antisense oligonucleotide is a splice switching oligonucleotide. In another embodiment, the antisense oligonucleotide is a gapmer. In certain cases, the antisense oligonucleotide is linked to the hinge region. In some cases, the therapeutic agent is a small molecule, peptide, enzyme, single chain antibody, nanobody, or nucleic acid (e.g., ASO) encapsulated within a liposome, polymeric nanocarrier, or lipid nanoparticle.

In another aspect, the disclosure provides a pharmaceutical composition comprising an antibody variable domain described herein, a chimeric molecule described herein, or any of the compositions described herein, and a pharmaceutically acceptable carrier.

In another aspect, the disclosure provides a nucleic acid or nucleic acids encoding the antibody variable domain, chimeric molecules, or compositions described herein.

In another aspect, the disclosure provides a vector or vectors comprising such nucleic acids.

In yet another aspect, the disclosure features a host cell comprising the above vector or vectors.

In another aspect, the disclosure provides a method of producing an antibody variable domain, chimeric molecule, or composition. The method comprises culturing the host cell described above in a cell culture medium under conditions that result in the expression of the antibody variable domain, chimeric molecule, or composition, and isolating the antibody variable domain, chimeric molecule, or composition from the cell culture medium. In some instances, the host cell is a CHO cell, a COS cell, or a HEK293 cell. In other instances, the host cell is a bacterial cell. In yet other instances, the host cell is a yeast cell (e.g., Pichia pastoris, Saccharomyces cerevisiae, Schizosaccharomyces pombe). In certain instances, the host cell is a fungal cell (e.g., Aspergillus).

In another aspect, the disclosure provides a method of producing a fusion polypeptide comprising an antibody variable domain described herein. The method comprises culturing a host cell containing a vector(s) that encode the fusion polypeptide in a cell culture medium under conditions that result in the expression of the fusion polypeptide, and isolating the fusion polypeptide from the cell culture medium.

In another aspect, the disclosure provides a method of treating Alzheimer's disease in a human subject in need thereof. The method comprises administering to the subject a chimeric molecule comprising an antibody variable domain described herein linked directly or via an intervening amino acid sequence to an antibody or antigen-binding fragment thereof that specifically binds human beta-amyloid. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of 12F6A (see, U.S. Pat. No. 8,906,367). In some cases, the antibody or antigen-binding fragment comprises the VH and VL of 12F6A.

In another aspect, the disclosure provides a method of treating progressive supranuclear palsy (PSP) disease in a human subject in need thereof. The method comprises administering to the subject a chimeric molecule comprising an antibody variable domain described herein linked directly or via an intervening amino acid sequence to an antibody or antigen-binding fragment thereof that specifically binds human beta-amyloid. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of 12F6A (see, U.S. Pat. No. 8,906,367). In some cases, the antibody or antigen-binding fragment comprises the VH and VL of 12F6A.

In another aspect, the disclosure features a method of treating a synucleinopathy (e.g., Parkinson' disease) in a human subject in need thereof. The method comprises administering to the subject a chimeric molecule comprising an antibody variable domain described herein linked directly or via an intervening amino acid sequence to an antibody or antigen-binding fragment thereof that specifically binds human alpha synuclein. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of 12F4 (see, U.S. Pat. No. 8,940,276) or 21D11 (see, U.S. Pat. No. 9,580,493). In some cases, the antibody or antigen-binding fragment comprises the VH and VL of 12F4 or 21D11.

In another aspect, the disclosure features a method of treating a tauopathy (e.g., Alzheimer's disease) in a human subject in need thereof. The method comprises administering to the subject a chimeric molecule comprising an antibody variable domain described herein linked directly or via an intervening amino acid sequence to an antibody or antigen-binding fragment thereof that specifically binds human tau. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of 6C5, 40E8 (see, U.S. Pat. No. 9,598,484), 4E4 (see, U.S. Pat. No. 9,605,059) or BMS-986168/hIPN002 (see, U.S. Pat. No. 9,447,180 and US Appl Publ. No. US2017/0174755A1). In some cases, the antibody or antigen-binding fragment comprises the VH and VL of 6C5, 40E8, 4E4, or BMS-986168/hIPN002.

In another aspect, the disclosure features a method of treating progressive supranuclear palsy in a human subject in need thereof. The method comprises administering to the subject a chimeric molecule comprising an antibody variable domain described herein linked directly or via an intervening amino acid sequence to an antibody or antigen-binding fragment thereof that specifically binds human tau. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of 6C5, 40E8 (see, U.S. Pat. No. 9,598,484), 4E4 (see, U.S. Pat. No. 9,605,059) or BMS-986168/hIPN002 (see, U.S. Pat. No. 9,447,180 and US Appl Publ. No. US2017/0174755A1). In some cases, the antibody or antigen-binding fragment comprises the VH and VL of 6C5, 40E8, 4E4, or BMS-986168/hIPN002.

In another aspect, the disclosure features a method of treating frontotemporal dementia in a human subject in need thereof. The method comprises administering to the subject a chimeric molecule comprising an antibody variable domain described herein linked directly or via an intervening amino acid sequence to an antibody or antigen-binding fragment thereof that specifically binds human TDP-43. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of 41D1, 14W3, 51C1 (see, U.S. Pat. No. 9,587,014), or a C9orf72 DPR. In some cases, the antibody or antigen-binding fragment comprises the VH and VL of 41D1, 14W3, 51C1, or an anti-C9orf72 DPR antibody (e.g., those disclosed herein).

In another aspect, the disclosure features a method of treating amyotrophic lateral sclerosis (ALS) in a human subject in need thereof. The method comprises administering to the subject a chimeric molecule comprising an antibody variable domain described herein linked directly or via an intervening amino acid sequence to an antibody or antigen-binding fragment thereof that specifically binds human SOD1, DR6, or a C9orf72 DPR. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of an antibody described in any one of U.S. Pat. Nos. 9,283,271; 8,759,029; or Broering et al., doi.org/10.1371/journal.pone.0061210.

In another aspect, the disclosure provides a method of treating multiple sclerosis in a human subject in need thereof. The method comprises administering to the subject a chimeric molecule comprising an antibody variable domain described herein linked directly or via an intervening amino acid sequence to an antibody or antigen-binding fragment thereof that specifically binds human LINGO-1. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of Li81 (see, U.S. Pat. No. 8,128,926) or Li113 (see, U.S. Pat. No. 8,058,406). In some cases, the antibody or antigen-binding fragment comprises the VH and VL of Li81 or Li113.

In another aspect, the disclosure provides a method of treating optic neuritis (e.g., AON) in a human subject in need thereof. The method comprises administering to the subject a chimeric molecule comprising an antibody variable domain described herein linked directly or via an intervening amino acid sequence to an antibody or antigen-binding fragment thereof that specifically binds human LINGO-1. In certain instances, the intervening amino acid sequence is a 3X(G4S) (SEQ ID NO:60), G4S (SEQ ID NO:5), GG, or G linker. In other instances, the intervening amino acid sequence comprises a hinge region of an antibody fused to a Fc moiety. In some instances, the antigen-binding fragment is a Fab. In some cases, the antibody or antigen-binding fragment comprises the six CDRs of Li81 (see, U.S. Pat. No. 8,128,926) or Li113 (see, U.S. Pat. No. 8,058,406). In some cases, the antibody or antigen-binding fragment comprises the VH and VL of Li81 or Li113.

In yet another aspect, the disclosure features a method of treating spinal muscular atrophy in a human subject in need thereof. The method comprising administering to the subject a chimeric molecule comprising an antibody variable domain described herein linked directly or via an intervening amino acid sequence to nusinersen (CAS Registry Number 1258984-36-9; UNII: 5Z9SP3X666). In some cases, nusinersen is encapsulated in a liposome, polymeric nanocarrier, or lipid nanoparticle. In other cases, the intervening amino acid sequence comprises a hinge region fused to a Fc moiety and nusinersen is linked to the hinge region, an engineered cysteine (e.g. S442C) in the chimeric molecule (e.g., hinge region), or a non-targeted site (e.g., using amine chemistry).

In another aspect, the disclosure features a method of assessing the lability of an antibody variable domain. The method involves providing an antibody variable domain. The antibody variable domain is added to a serum sample to create a mixture. The mixture is incubated. The antibody variable domain is purified and peptide mapping is performed.

In another aspect, the disclosure features a method of screening for a stabilized form of FCS. The method involves providing an antibody variable domain comprising a FC5 variant that differs from SEQ ID NO:1 at one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) amino acids, or comprises a n amino acid sequence that is at least 60%, 65%, 70%, 75%, 80%, 85%, 90%, or 95% identical to SEQ ID NO:1. The antibody variable domain is added to a serum sample to create a mixture. The mixture is incubated. The antibody variable domain is purified and peptide mapping is performed. An antibody variable domain that exhibits increased peptide recovery is selected as a stabilized form of FC5.

In some instances of the above two aspects, the serum sample is rat serum. In other instances, the serum sample is human serum. In certain instances, the antibody variable domain is in the serum at a concentration of about 0.01 to 1 mg/mL. In certain instances, the antibody variable domain is in the serum at a concentration of about 0.05 to 0.5 mg/mL. In certain instances, the antibody variable domain is in the serum at a concentration of about 0.05 mg/mL. In certain instances, the antibody variable domain is in the serum at a concentration of about 0.1 mg/mL. In certain instances, the antibody variable domain is in the serum at a concentration of about 0.5 mg/mL. In some cases, the mixture is incubated at about 25 to 37° C. for about 10 to 100 hours. In one case, the mixture is incubated at about 37° C. for about 70 hours. In one embodiment, the antibody variable domain comprises an FC5 variant. In some cases, the FC5 variant is a humanized FC5. In another case, the FC5 variant comprises a disulfide-stabilized FC5. In yet another case, the FC5 variant comprises a humanized and disulfide-stabilized FC5.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 provides the amino acid sequence of the wild type FC5 single domain antibody (SEQ ID NO:1). The position of each amino acid in the FC5 amino acid sequence is also provided (numbering according to primary sequence). The CDRs are at positions 23-35 (CDR1), 50-66 (CDR2), and 97-111 (CDR3). CDRs 1 and 3 are defined according to North et al. (North, B., Lehmann, A. & Dunbrack, R. L., Jr, “A New Clustering of Antibody CDR Loop Conformations”, J. Mol. Biol., 406:228-256 (2011)) to account for structural variability, and CDR2 is defined as a union of the definitions of Kabat et al. (Kabat, E. A., Wu, T. T., Perry, H. M., Gottesman, K. S. & Foeller, C. (1991). Sequences of Proteins of Immunological Interest, 5th edit. National Institutes of Health, Bethesda, Md.) and North et al. to account for both structural and sequence variability.

FIG. 2 provides the amino acid sequences of the light chain of Li81 (SEQ ID NO:2), the heavy chain of FC5-3X(G4S)-HC Li81 (SEQ ID NO:3), and the light chain of FC5-3X(G4S)-LC Li81 (SEQ ID NO:4). These are the sequences of the constructs used to produce constructs 3014, 3015, and 3016. The signal peptide sequence is shown in lighter font.

FIG. 3 shows the characterization of FC5-(G₄S)3-Li81 samples by SDS-PAGE. Samples were subjected to SDS-PAGE on 4-20% Tris-glycine gradient gels from Invitrogen. Non-reduced samples were heated at 95° C. for 2 min prior to analysis (left panel). Reduced samples were treated with sample buffer containing 2% 2-mercaptoethanol and heated (right panel). Lane 1, FC5 Li81 HC, wt LC (4D #3014); lane 2, Li81 wt HC, FC5 Li81 LC (4D #3015); lane 3, FC5 Li81 HC, FC5 Li81 LC (4D #3016); lane S, high molecular weight markers.

FIG. 4 depicts the deconvoluted mass spectra of the light (top) and heavy (bottom) chains for the reduced samples of construct 3014.

FIG. 5 shows the deconvoluted mass spectra of the heavy and light chains for the reduced samples of construct 3015.

FIG. 6 depicts the deconvoluted mass spectra of the heavy and light chains for the reduced samples of construct 3016.

FIG. 7 illustrates the characterization of FC5-(G4S)1-Li81 samples by SDS-PAGE. Samples were subjected to SDS-PAGE on 4-20% Tris-glycine gradient gels from Invitrogen. Non-reduced samples were heated at 95° C. for 2 min prior to analysis. Left panel: lane 1, FC5 (G₄S)1-Li81 (ID #3438); lane 2, FC5-Fc (G₄S)1-Li81 long hinge (ID #3440); lane 3, FC5-Fc (G₄S)1-Li81 short hinge (ID #3441); lane 4 heterodimer ID #3442; lane 5 heterodimer ID #3443; lane 6 heterodimer ID #3444; lane 7 heterodimer ID #3443 (batch2); lane S, high molecular weight markers. Right panel, purification of heterodimer ID #3443 by SEC: lane 1, column load; lanes 2-11, sequential elution fractions; lane S, high molecular weight markers.

FIG. 8 provides the amino acid sequences of the constructs used to produce the FC5-(G₄S)1-Li81 constructs. The light chain of Li81 (SEQ ID NO:2), the heavy chain of FC5-(G₄S)1-Li81 huIgG1 agly (SEQ ID NO:7), the light chain of FC5-Fc-(G₄S)1-Li81 huIgG1 agly long hinge (SEQ ID NO:8), and FC5-Fc-(G₄S)1-Li81 huIgG1 agly short hinge (SEQ ID NO:9). The signal peptide sequence is shown in lighter font.

FIG. 9 depicts a deconvoluted mass spectra of heavy chains for the FC5-(G₄S)1-Li81 constructs. Li81/FC5 variant 1-FC5-(G₄S)1-Li81; Li81/FC5 Variant 3-FC5-Fc-(G₄S)1-Li81 long hinge; Li81/FC5 Variant 4 FC5-Fc-(G₄S)1-Li81 short hinge; Li81/FC5 Variant 5 dual-monovalent FC5-Fc-(G₄S)1-Li81 long hinge; Li81/FC5 Variant 6 dual-monovalent FC5-Fc-(G₄S)1-Li81 short hinge; and Li81/FC5 Variant 7 monoFab FC5-Fc-(G₄S)1-Li81 short hinge samples.

FIG. 10 shows an assessment of the potency of FC5-(G₄S)1-Li81 constructs. The apparent affinities of FC5-(G₄S)1-Li81 samples for LINGO-1 were measured by a direct binding ELISA. Data are plotted as absorbance at 405 nm versus concentration.

FIG. 11 depicts the characterization of FC5-Li81 samples in an in vitro BBB cell culture assay. The levels of antibodies in the bottom chamber (15, 30, 60 and 90 min) were determined by MRM-ILIS. Values are Means±SD of Pₐₚₚ.

FIG. 12 illustrates the effects of FC5-Li81 fusions cross-linked with dalargin (Dal) on thermal hyperalgesia in Hargreaves model of inflammatory pain. Top panel: Latency of withdrawal of control intact paw or inflamed paw to a thermal stimulus was measured after a single intravenous (i.v.) dose of 13 mg/kg of the test molecules, or PBS at 15 min intervals over 4 h. Data are shown as means (2 animals/group; error bars denote individual measurements). Bottom panel: Integrated area under the curve (AUC) data from top panel expressed as percentage maximum possible effect (MPE) of control paw.

FIG. 13A shows the effects of FC5-Li81 fusions cross-linked with Dal on thermal hyperalgesia in Hargreaves model of inflammatory pain. Analgesic effect achieved with a single i.v. injection of escalating doses of Li81 and FC5-Li81. Data are the integrated AUC over the duration of response (4 h) expressed as percentage MPE of control paw (means±standard deviations of 2 or 5 animals/group as noted).

FIG. 13B shows the data from FIG. 13A plotted as the analgesic effect of FC5-Li81 (3438) and Li81, crosslinked with dalargin, versus the i.v. dose.

FIG. 14 provides a serum and CSF PK assessment of FC5-Li81. Samples were administered IV via the tail vein at 20, 65, and 200 nmol/kg each, and antibody levels at indicated time points after injection were determined in serum (top panels) and CSF (middle panels) samples from the cisterna magna. Paired CSF/serum ratios (%) at each time point are plotted in bottom panel. Results are the mean of 2 separate animals.

FIG. 15 illustrates the analysis of the PK of FC5-(G₄S)1-Li81 in rats. Serum and CSF levels were quantified by mass spectrometry. Data are presented as a ratio of CSF and serum levels.

FIG. 16 depicts the characterization of FC5-(G₄S)1-Li81 samples by SDS-PAGE. Samples were subjected to SDS-PAGE on 4-20% tris-glycine gradient gels from Invitrogen. Non-reduced samples were treated with 5 mM N-ethyl maleimide for 5 min at room temperature, diluted with Laemmli non-reducing sample buffer and heated at 95° C. for 2 min prior to analysis. Reduced samples were treated with sample buffer containing 2% 2-mercaptoethanol and heated. Lane 1. High molecular weight markers Lane 2. FC5-(G₄S)1-Li81 (dosing preparation) Lane 3. FC5-(G₄S)1-Li81 purified from rat serum after 72 h Lane 4. FC5-(G₄S)1-Li81-HC purified from rat serum spike sample at 100 μg/mL.

FIG. 17 shows the characterization of FC5-(G₄S)1-Li81 samples by size exclusion chromatography. Samples (25 μg in 300 μL of column buffer) were subjected to SEC at room temperature on a Superdex 200 HR10/30 FPLC column (GE Healthcare) using 20 mM sodium phosphate, 150 mM NaCl, pH 7.2 as the mobile phase. The column was run at 0.3 mL/min. The column effluent was monitored by UV detection at 280 nm. Arrows denote the time of injection. SEC/light scattering was carried out on a BioSep-SEC-53000 column, 300×7.8 mm (Phenomenex) in 20 mM sodium phosphate, 150 mM NaCl, pH 7.2 at a flow rate of 0.6 mL/min on a Waters Alliance instrument (Waters 2790, MA). Static light scattering was synchronized with SEC and measured on-line using a Precision PD2100 Detector (Precision Detectors, MA). Molecular weights were calculated using Discovery 32 Light Scattering Analysis Software.

FIG. 18 displays the characterization of FC5-(G₄S)1-Li81 samples in an in vitro BBB cell culture assay. The levels of antibodies in the bottom chamber (15, 30, 60 and 90 min) were determined by MRM-ILIS. Values are Means±SD of Papp.

FIG. 19 shows the mass spectra of native of FC5-(G₄S)1-Li81 samples. Deconvoluted mass spectra of the native FC5(G₄S)1Li81 dosing solution, FC5-(G₄S)1-Li81 spike, and FC5-(G₄S)1-Li81 72 h samples.

FIG. 20 shows the mass spectra of FC5-Li81 HC samples. Deconvoluted mass spectra of the native FC5(G₄S)1Li81 dosing solution, FC5-(G₄S)1-Li81 spike, and FC5-(G₄S)1-Li81 72 h samples.

FIG. 21 shows the mass spectra of FC5-Li81 LC samples. Deconvoluted mass spectra of the native FC5(G₄S)1Li81 dosing solution, FC5-(G₄S)1-Li81 spike, and FC5-(G₄S)1-Li81 72 h samples.

FIG. 22 depicts EndoLysC/trypsin peptide maps of FC5-Li81 samples. The UV traces, monitored at 214 nm, of the non-reduced and reduced Lys-C/tryptic disulfide maps of FC5(G₄S)1Li81 (control), FC5-(G₄S)1-Li81-HC spike and FC5-(G₄S)1-Li81-HC 72 h.

FIG. 23 shows the recovery of the heavy chain peptides in the non-reduced and reduced peptide maps. % recovery (y-axis) was normalized to the corresponding peptides in the control sample. HC peptides that were characterized are indicated on the x-axis. Poor recovery peptide, HC 215-249; hydrophobic peptides, HC 171-192 and HC 276-338.

FIG. 24 depicts the recovery of the light chain peptides in the non-reduced and reduced peptide maps. % recovery (y-axis) was normalized to the corresponding peptides in the control sample. LC peptides that were characterized are indicated on the x-axis.

FIG. 25 illustrates the design of humanized FC5. Sequence alignment and a computational structure model comparing FC5 (SEQ ID NO:1) and humanization variants H12 (SEQ ID NO:11) and H62 (SEQ ID NO:15). Light shading of side chains in the models and amino acid one-letter codes in the alignment indicates camelid origin, and dark shading indicates human origin. H12 has a fully humanized patch (dashed outline) that corresponds to the VH/VL interface in a conventional antibody. This interface is covered by the light chain in a conventional antibody, but is exposed in single-domain antibodies. The M34T mutation is introduced to avoid methionine oxidation.

FIG. 26 depicts the characterization of FC5-hFc humanization variants by denaturing microfluidic capillary electrophoresis. 1) H11. 2) H12. 3) H31. 4) H32. 5) H61. 6) H62. 7) H71. 8) H72. Left: non-reduced samples. Right: reduced samples. Molecular weight markers are indicated in kDa on both sides. Using the instrument software, the electropherograms are displayed in 2D as a virtual gel using the lower marker (LM) for alignment. SP=system peak

FIG. 27 shows the deconvoluted intact mass spectra of reduced FC5-hFc humanization variant samples. H11: predicted mass 38553.7 Da, observed mass 38551. H12: predicted mass 38523.6 Da, observed mass 38521 Da. H31: predicted mass 38548.6 Da, observed mass 38544 Da. H32: predicted mass 38518.6 Da, observed mass 38516 Da. H61: predicted mass 38649.7 Da, observed mass 38645 Da. H62: predicted mass 38619.6 Da, observed mass 38617 Da. H71: predicted mass 38677.8 Da, observed mass 38674 Da. H72: predicted mass 38647.7 Da, observed mass 38645 Da.

FIG. 28 shows the results of analytical size exclusion chromatography of humanization variants of FC5-hFc. Variant H61, H62, and H71 eluted as homogeneous peaks at ˜17.3 min, whereas other variants show delayed elution times in the order listed. Data are normalized for constant peak height.

FIG. 29 is a characterization of humanization variants of FC5-hFc in an in vitro rat BBB cell culture transwell assay. The levels of antibodies in the bottom chamber (15, 30, and 60 min) were determined by MRM and used to calculate the permeability values. Values are Means±SD of P_{APP}.

FIG. 30 shows the effects of FC5-hFc humanization variants cross-linked with Dalargin on thermal hyperalgesia in Hargreaves model of inflammatory pain. Left panel: Latency of withdrawal of control intact paw or inflamed paw to a thermal stimulus was measured after a single i.v. dose of ˜5 mg/kg of the test molecules, or PBS at 15 min intervals over 4 h. Data are shown as means (sd for 2 animals/group). Right panel: Integrated response (AUC) data from left panel expressed as percentage MPE of control paw.

FIG. 31 shows the engineering of disulfide bonds within FC5 to stabilize its structure. Confirmation of proper distances between positions for cysteine mutations and minimal disruption of protein interior was achieved through computational modeling. The top panel shows the positions of the cysteines indicated and the bottom panel shows the models with the disulfides formed.

FIG. 32 depicts the characterization of FC5-(G₄S)1-Li81 samples by SDS-PAGE. Samples were subjected to SDS-PAGE on 4-20% Tris-glycine gradient gels from Invitrogen. Non-reduced samples were heated at 95° C. for 2 min prior to analysis. Reduced samples were treated with sample buffer containing 2% 2-mercaptoethanol and heated. Lane 1, 4D 4662; lane 2, 4D 4663; lane 3, 4D 4664; lane 4, 4D 4665; lane 5, 4D 4666; lane 6, 4D 4667; lane 7, 4D 4668; lane 8, 4D 4669; lane 9, 4D 4670; lane 10, high molecular weight markers.

FIG. 33 displays the characterization of disulfide stabilized FC5-(G₄S)1-Li81 samples in the in vitro BBB cell culture transwell assay. The levels of antibodies in the bottom chamber (15, 30, 60 and 90 min) were determined by MRM-ILIS. Values are Means±SD of P_{APP}.

FIG. 34 is an assessment of the susceptibility of FC5-(G₄S)1-Li81 samples to proteolysis by SDS-PAGE. Samples were subjected to SDS-PAGE on 4-20% Tris-glycine gradient gels from Invitrogen. Non-reduced samples were heated at 95° C. for 2 min prior to analysis. Reduced samples were treated with sample buffer containing 2% 2-mercaptoethanol and heated. Lane 1, H32(T34C/V79C)-G4S-Li81; lane 2, H32(T34C/V79C)-G4S-Li81+pepsin; lane 3, H32(T33C/T103C)-G4S-Li81; lane 4, H32(T33C/T103C)-G4S-Li81+pepsin; lane 5, Li81; lane 6, Li81+pepsin; lane 7, FC5-H32-G4S-Li81; lane 8, FC5-H32-G4S-Li81+pepsin; lane 9, high molecular weight markers.

FIG. 35 is an assessment of the susceptibility of FC5-(G₄S)1-Li81 samples to proteolysis by SDS-PAGE. Samples were subjected to SDS-PAGE on 4-20% Tris-glycine gradient gels from Invitrogen. Non-reduced samples were heated at 95° C. for 2 min prior to analysis. Reduced samples were treated with sample buffer containing 2% 2-mercaptoethanol and heated. Lane 1, FC5-H62(S49C/I70C)-G4S-Li81; lane 2, FC5-H62(S49C/I70C)-G4S-Li81+Pepsin; lane 3, FC5-H62(T34C/V79C)-G4S-Li81+pepsin; lane 4, FC5-H62(T33C/S102C)-G4S-Li81+pepsin; lane 5, FC5-H62(T33C/T103C)-G4S-Li81+pepsin; lane 6, FC5-H62(T33C/A104C)-G4S-Li81+pepsin; lane 7, FC5-H62(T33C/T105C)-G4S-Li81+pepsin; lane 8, FC5-H32-Li81-G4S-Li81+pepsin; lane 9, Li81+pepsin; lane 10, H62-FC5-Fc; lane 11, H62-FC5-Fc+pepsin; lane 12, H32-FC5-Fc; lane 13, H32-FC5-Fc+pepsin; lane 14, High molecular weight marker. To facilitate the analysis of the samples, the bottom panel shows an expanded view of lanes 1-9 from the 100-150 kDa region of the non-reduced SDS-PAGE.

FIG. 36 displays the deconvoluted mass spectra of the heavy chains for the reduced samples 4662, 4665, 4667, and 4668.

FIG. 37 displays the deconvoluted mass spectra of the light chains for the reduced samples 4662, 4665, 4667, and 4668.

FIG. 38 is a depiction of the recovery of the heavy chain peptides in the non-reduced and reduced peptide maps. % recovery (y-axis) was normalized to the corresponding peptides in the control sample. HC peptides that were characterized are indicated on the x-axis. Poor recovery peptide, HC 215-249; hydrophobic peptides, HC 171-192 and HC 276-338.

FIG. 39 is a depiction of the recovery of the light chain peptides in the non-reduced and reduced peptide maps. % recovery (y-axis) was normalized to the corresponding peptides in the control sample. LC peptides that were characterized are indicated on the x-axis.

FIG. 40 depicts a serum and CSF PK assessment of FC5-Li81. Samples were administered IV via the tail vein at 65 nmol/kg each, and antibody levels at indicated time points after injection were determined in serum (top panel) and CSF (middle panel) samples from the cisterna magna. Paired CSF/serum ratios (%) at each time point are plotted in bottom panel. Results are the mean of 2 separate animals.

FIG. 41 shows a serum PK assessment of the three disulfide stabilized FC5-Li81 variants showing greatest improvements in in vitro stability. Samples were administered i.v. via the tail vein at 65 nmol/kg each, and antibody levels at indicated time points after injection were determined in serum. Results are the mean of 2 separate animals.

FIG. 42A depicts the assessment of brain levels of the three disulfide-stabilized FC5-Li81 variants showing greatest improvements in in vitro stability. Samples were administered i.v. via the tail vein at 65 nmol/kg each, and antibody levels at indicated time points after injection were determined in serum samples.

FIG. 42B depicts the assessment of CSF levels of the three disulfide-stabilized FC5-Li81 variants showing greatest improvements in in vitro stability. Samples were administered i.v. via the tail vein at 65 nmol/kg each, and antibody levels at indicated time points after injection were determined in CSF samples from the cisterna magna.

FIG. 42C shows a plot of the paired CSF/serum ratios (%) of the three disulfide-stabilized FC5-Li81 variants at each time point. Results are the mean 2 separate animals.

FIG. 42D depicts the brain levels of the three disulfide-stabilized FC5-Li81 variants measured at the 24 h timepoint.

FIG. 43A shows a serum and CSF PK assessment of FC5-Li81 in rats. Samples were administered i.v. via the tail vein at 65 nmol/kg each, and antibody levels at indicated time points after injection were determined in serum samples by MRM. Results are the mean of 2-3 separate animals. Schematics of molecular designs are shown to the left.

FIG. 43B shows a serum and CSF PK assessment of FC5-Li81 in rats. Samples were administered IV via the tail vein at 65 nmol/kg each, and antibody levels at indicated time points after injection were determined in CSF samples from the cisterna magna. Results are the mean of 2-3 separate animals. Schematics of molecular designs are shown to the left.

FIG. 43C shows paired CSF/serum ratios (%) from the data plotted in FIGS. 43A and 43B. Results are the mean of 2-3 separate animals. Schematics of molecular designs are shown to the left.

FIG. 44 shows a serum and CSF PK assessment of FC5-H62(T33C/A104C)-Li81 (solid lines) versus Li81 (dashed lines) in cynomolgus monkeys (n=12). Test articles were administered via single IV bolus injection at 65 nmol/kg, and antibody levels at indicated time points after injection were determined by nanoLC-MRM in samples of serum (top panel) and CSF (middle panel). Paired CSF/serum ratios are also plotted (bottom panel).

FIG. 45A shows a graph of FC5-H62(T33C/A104C)-12F6A amyloid beta direct binding ELISA. Synthetic amyloid beta (aa 1-40) peptide was coated on ELISA plates, exposed to several dilutions of test reagents, and antibody binding at the indicated concentrations was detected with an HRP conjugated anti-hIgG secondary, measured by absorbance at 450 nm. EC50 values calculated from fitting the data to a sigmoidal curve are indicated.

FIG. 45B shows SV40-immortalized brain endothelial cell (svARBEC) transwell assay results recorded by measurement of bottom-well antibody concentrations by MRM (means±SD of apparent permeability, P_{APP}, for n=3 replicates). Control antibody in each well was anti-HEL murine IgG1. hFC5.2 is FC5-H62(T33C/A104C).

FIG. 45C shows human iPSC-derived brain endothelial cell transwell assay results recorded by measurement of bottom-well antibody concentrations by MRM (means±SD of apparent permeability, P_{APP}, for n=3 replicates). Control antibody in each well was anti-HEL murine IgG1. hFC5.2 is FC5-H62(T33C/A104C).

FIG. 46A is a graph showing alpha-synuclein direct binding ELISA. Human alpha-synuclein was coated on ELISA plates, treated with several dilutions of test reagents, and antibody binding at the indicated concentrations was detected with an HRP conjugated anti-hIgG secondary, measured by absorbance at 450 nm. EC50 values calculated from fitting the data to a sigmoidal curve are indicated. hFC5.2 is FC5-H62(T33C/A104C).

FIG. 46B is a bar graph of SVARBEC transwell assay results recorded by measurement of bottom-well antibody concentrations by MRM. Data are plotted as a ratio of P_{App} for test antibody over in-well anti-HEL mIgG1 control (means±SD for n=3 replicates); P_{APP} for anti-HEL was <1.5×10′ cm/min for all wells. hFC5.2 is FC5-H62(T33C/A104C).

FIG. 47A shows the results of Tau direct binding ELISA. Human tau was coated on ELISA plates, treated with several dilutions of test reagents, and bound antibody was detected with an HRP conjugated anti-hIgG secondary, measured by absorbance at 450 nm. EC50 values calculated from fitting the data to a sigmoidal curve are indicated. hFC5.2 is FC5-H62(T33C/A104C).

FIG. 47B is a bar graph of SVARBEC transwell assay results recorded by measurement of bottom-well antibody concentrations by MRM. Data are plotted as a ratio of P_{App} for test antibody over in-well anti-HEL mIgG1 control (means±SD for n=3 replicates); P_{App} for anti-HEL was <1.5×10′ cm/min for all wells. hFC5.2 is FC5-H62(T33C/A104C).

FIG. 48 is a validation of reduced susceptibility of disulfide engineered FC5-antibody fusions to pepsin digest. Left: SDS-PAGE analysis of FC5-H62(T33C/A104C) fused to Li81 hIgG1 agly, 12F4 hIgG1, and 12F4 hIgG1 agly, with and without pepsin digest for 1 h at 37° C. FC5-H32-Li81 hIgG1 agly shows increased digestion as indicated by more lower molecular weight fragments (arrows). Right: SDS-PAGE analysis of pepsin digests (2.5 h at 37° C.) of FC5-H62(T33C/A104C) or FC5-H32 fused to Li81 hIgG1 agly and 12F6A hIgG1.

FIG. 49 is a schematic depiction of non-limiting illustrative bivalent FC5 fusion formats.

### DETAILED DESCRIPTION

Delivery of drugs to the central nervous system (CNS) has been a central problem in the treatment of neurological diseases because the blood brain barrier (BBB) is a barricade preventing most therapeutic molecules from entering the brain. Without access to the brain, the therapeutic potential of these molecules can be compromised or eliminated.

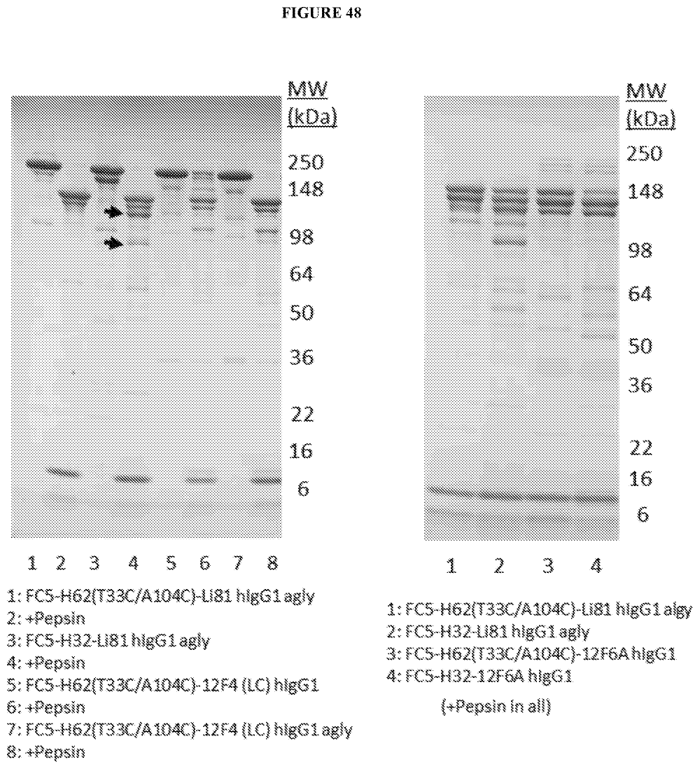

This disclosure is based, in part, on molecules comprising humanized FC5 as well as disulfide-engineered FC5. These molecules are useful to transport a “cargo” of interest such as an antibody, an antigen-binding fragment, a peptide, an enzyme, and a nucleic acid (e.g., an antisense oligonucleotide) to the CNS. In some embodiments, these molecules transcytose across the BBB. In some embodiments, these molecules can bind directly or indirectly to human TMEM30A (UniProtKB-Q9NV96). In certain embodiments, these molecules bind better to cells that express TMEM30A than to cells that do not express (or that are modified to not express) TMEM30A. In some embodiments, these molecules have reduced lability in human serum as compared to wild type FC5 (SEQ ID NO:1). Each of the FC5-therapeutic agent (e.g., antibody) formats including monovalent FC5 formats schematically illustrated in FIG. 48 are encompassed by the present disclosure

### Humanized FC5

FC5 is a camelid single domain antibody that can transcytose across the BBB and can deliver bioactive molecules to the CNS (see, e.g., Muruganandam et al., FASEB J., 16:240-242 (2002); Abulrob et al., J Neurochem., 95:1201-1214 (2005); Haqqani et al., Mol. Pharmaceutics, 10:1542-1556 (2013); Farrington et al., FASEB J., 28:4764-4778 (2014); U.S. Pat. No. 9,676,849). The amino acid sequence of the wild type FC5 is provided in SEQ ID NO:1.

FC5 is species cross-reactive and it can bind to rat, mouse, and human brain endothelial cells. This disclosure features, inter alia, humanized FC5 antibody variable domains. These humanized FC5 antibody variable domains reduce the potential risk of human immunogenicity of FC5, while maintaining solubility and stability. The FC5 sequence antibody variable domains have varying degrees of homology to the human VH3 consensus framework.

The camelid VHH domain framework is highly homologous to the human VH3 family of heavy chain variable domain frameworks. To construct a human VH3 consensus framework, a multiple sequence alignment (MSA) was obtained with Clustal Omega (www.ebi.ac.uk/Tools/msa/clustalo/), which summarized all human functional and ORF VH3 genes deposited in IMGT as of Aug. 25, 2014. The consensus framework was defined as the sequence of the highest-frequency amino acid at each position of the MSA, and combined with the first allele of the human JH4 gene (IGHJ4*01, IMGT nomenclature) to obtain the framework of a complete VH domain. In framework regions 1, 2 and 3, the consensus framework is identical with that of the fourth allele of the human VH3 gene IGHV3-23*04. FC5 has 15 framework residues that differ from the human VH3 consensus framework. Camelid residues 37, 44, 45, and 47 are on the surface that corresponds to the VH/VL interface in human IgG. These residues can strongly affect solubility, expression levels, or binding affinity of the single-domain antibody, but these effects also depend on the CDRs of each individual single-domain antibody.

The complementarity determining regions (CDRs) of wild type FC5 are located at positions 23-35 (CDR1), 50-66 (CDR2), and 97-111 (CDR3) of SEQ ID NO:1 and are provided below:

Wild type FC5 CDR1:
(SEQ ID NO: 47)
AASGFKITHYTMG
Wild type FC5 CDR2:
(SEQ ID NO: 50)
RITWGGDNTFYSNSVKG
Wild type FC5 CDR3:
(SEQ ID NO: 51)
AAGSTSTATPLRVDY.

In some embodiments, the FC5 polypeptides of this disclosure have a CDR1 selected from one of the amino acid sequences listed below.

(SEQ ID NO: 57)
AASGFKITHYTTG
(SEQ ID NO: 48)
AASGFKITHYTCG
(SEQ ID NO: 58)
AASGFKITHYCMG
(SEQ ID NO: 49)
AASGFKITHYCTG

In certain embodiments, the FC5 polypeptides of this disclosure have a CDR3 selected from one of the amino acid sequences listed below.

(SEQ ID NO: 52)
AAGSTCTATPLRVDY
(SEQ ID NO: 53)
AAGSTSCATPLRVDY
(SEQ ID NO: 54)
AAGSTSTCTPLRVDY
(SEQ ID NO: 55)
AAGSTSTACPLRVDY

In certain embodiments, the antibody variable domain of this disclosure comprises a polypeptide comprising a CDR1 comprising or consisting of the amino acid sequence set forth in SEQ ID NO:47 with two or fewer (e.g., 2, 1, 0) amino acid substitutions, a CDR2 comprising or consisting of the amino acid sequence set forth in SEQ ID NO:50 with two or fewer amino acid substitutions, and a CDR3 comprising or consisting of the amino acid sequence set forth in SEQ ID NO:51 with two or fewer amino acid substitutions. In some embodiments, the antibody variable domain of this disclosure comprises a polypeptide comprising a CDR1 comprising or consisting of the amino acid sequence set forth in SEQ ID NO:47 with two or fewer (e.g., 2, 1, 0) amino acid substitutions, a CDR2 comprising or consisting of the amino acid sequence set forth in SEQ ID NO:50, and a CDR3 comprising or consisting of the amino acid sequence set forth in SEQ ID NO:51 with two or fewer amino acid substitutions. In certain embodiments, the antibody variable domain of this disclosure comprises a polypeptide comprising a CDR1 comprising or consisting of the amino acid sequence set forth in SEQ ID NO:47 with one or no amino acid substitution, a CDR2 comprising or consisting of the amino acid sequence set forth in SEQ ID NO:50 with one or no amino acid substitution, and a CDR3 comprising or consisting of the amino acid sequence set forth in SEQ ID NO:51 with one or no amino acid substitution. In some embodiments, the antibody variable domain of this disclosure comprises a polypeptide comprising a CDR1 comprising or consisting of the amino acid sequence set forth in SEQ ID NO:47 with one or no amino acid substitution, a CDR2 comprising or consisting of the amino acid sequence set forth in SEQ ID NO:50, and a CDR3 comprising or consisting of the amino acid sequence set forth in SEQ ID NO:51 with one or no amino acid substitution. The above-described antibody variable domains can be humanized. In some instances, in all of the above embodiments, the amino acid substitutions occur at one or more (e.g., 1, 2, 3, 4, 5, 6) of positions 33, 34, 102, 103, 104, or 105 of SEQ ID NO:1. In some embodiments, these molecules transcytose across the BBB. In some embodiments, these molecules can bind directly or indirectly to human TMEM30A (UniProtKB-Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In certain embodiments, the antibody variable domain of this disclosure comprises a polypeptide comprising a CDR1 comprising or consisting of the amino acid sequence set forth in SEQ ID NO:57, a CDR2 comprising or consisting of the amino acid sequence set forth in SEQ ID NO:50, and a CDR3 comprising or consisting of the amino acid sequence set forth in SEQ ID NO:52. In other embodiments, the antibody variable domain of this disclosure comprises a polypeptide comprising a CDR1 comprising or consisting of the amino acid sequence set forth in SEQ ID NO:57, a CDR2 comprising or consisting of the amino acid sequence set forth in SEQ ID NO:50, and a CDR3 comprising or consisting of the amino acid sequence set forth in SEQ ID NO:53. In other embodiments, the antibody variable domain of this disclosure comprises a polypeptide comprising a CDR1 comprising or consisting of the amino acid sequence set forth in SEQ ID NO:57, a CDR2 comprising or consisting of the amino acid sequence set forth in SEQ ID NO:50, and a CDR3 comprising or consisting of the amino acid sequence set forth in SEQ ID NO:54. In other embodiments, the antibody variable domain of this disclosure comprises a polypeptide comprising a CDR1 comprising or consisting of the amino acid sequence set forth in SEQ ID NO:57, a CDR2 comprising or consisting of the amino acid sequence set forth in SEQ ID NO:50, and a CDR3 comprising or consisting of the amino acid sequence set forth in SEQ ID NO:55. In some embodiments, these molecules transcytose across the BBB. In some embodiments, these molecules can bind directly or indirectly to human TMEM30A (UniProtKB-Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In certain embodiments, the antibody variable domain of this disclosure comprises a polypeptide comprising a CDR1 comprising or consisting of the amino acid sequence set forth in SEQ ID NO:48, a CDR2 comprising or consisting of the amino acid sequence set forth in SEQ ID NO:50, and a CDR3 comprising or consisting of the amino acid sequence set forth in SEQ ID NO:52. In other embodiments, the antibody variable domain of this disclosure comprises a polypeptide comprising a CDR1 comprising or consisting of the amino acid sequence set forth in SEQ ID NO:48, a CDR2 comprising or consisting of the amino acid sequence set forth in SEQ ID NO:50, and a CDR3 comprising or consisting of the amino acid sequence set forth in SEQ ID NO:53. In other embodiments, the antibody variable domain of this disclosure comprises a polypeptide comprising a CDR1 comprising or consisting of the amino acid sequence set forth in SEQ ID NO:48, a CDR2 comprising or consisting of the amino acid sequence set forth in SEQ ID NO:50, and a CDR3 comprising or consisting of the amino acid sequence set forth in SEQ ID NO:54. In other embodiments, the antibody variable domain of this disclosure comprises a polypeptide comprising a CDR1 comprising or consisting of the amino acid sequence set forth in SEQ ID NO:48, a CDR2 comprising or consisting of the amino acid sequence set forth in SEQ ID NO:50, and a CDR3 comprising or consisting of the amino acid sequence set forth in SEQ ID NO:55. In some embodiments, these molecules transcytose across the BBB. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In certain embodiments, the antibody variable domain of this disclosure comprises a polypeptide comprising a CDR1 comprising or consisting of the amino acid sequence set forth in SEQ ID NO:58, a CDR2 comprising or consisting of the amino acid sequence set forth in SEQ ID NO:50, and a CDR3 comprising or consisting of the amino acid sequence set forth in SEQ ID NO:52. In other embodiments, the antibody variable domain of this disclosure comprises a polypeptide comprising a CDR1 comprising or consisting of the amino acid sequence set forth in SEQ ID NO:58, a CDR2 comprising or consisting of the amino acid sequence set forth in SEQ ID NO:50, and a CDR3 comprising or consisting of the amino acid sequence set forth in SEQ ID NO:53. In other embodiments, the antibody variable domain of this disclosure comprises a polypeptide comprising a CDR1 comprising or consisting of the amino acid sequence set forth in SEQ ID NO:58, a CDR2 comprising or consisting of the amino acid sequence set forth in SEQ ID NO:50, and a CDR3 comprising or consisting of the amino acid sequence set forth in SEQ ID NO:54. In other embodiments, the antibody variable domain of this disclosure comprises a polypeptide comprising a CDR1 comprising or consisting of the amino acid sequence set forth in SEQ ID NO:58, a CDR2 comprising or consisting of the amino acid sequence set forth in SEQ ID NO:50, and a CDR3 comprising or consisting of the amino acid sequence set forth in SEQ ID NO:55. In some embodiments, these molecules transcytose across the BBB. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In certain embodiments, the antibody variable domain of this disclosure comprises a polypeptide comprising a CDR1 comprising or consisting of the amino acid sequence set forth in SEQ ID NO:49, a CDR2 comprising or consisting of the amino acid sequence set forth in SEQ ID NO:50, and a CDR3 comprising or consisting of the amino acid sequence set forth in SEQ ID NO:52. In other embodiments, the antibody variable domain of this disclosure comprises a polypeptide comprising a CDR1 comprising or consisting of the amino acid sequence set forth in SEQ ID NO:49, a CDR2 comprising or consisting of the amino acid sequence set forth in SEQ ID NO:50, and a CDR3 comprising or consisting of the amino acid sequence set forth in SEQ ID NO:53. In other embodiments, the antibody variable domain of this disclosure comprises a polypeptide comprising a CDR1 comprising or consisting of the amino acid sequence set forth in SEQ ID NO:49, a CDR2 comprising or consisting of the amino acid sequence set forth in SEQ ID NO:50, and a CDR3 comprising or consisting of the amino acid sequence set forth in SEQ ID NO:54. In other embodiments, the antibody variable domain of this disclosure comprises a polypeptide comprising a CDR1 comprising or consisting of the amino acid sequence set forth in SEQ ID NO:49, a CDR2 comprising or consisting of the amino acid sequence set forth in SEQ ID NO:50, and a CDR3 comprising or consisting of the amino acid sequence set forth in SEQ ID NO:55. In some embodiments, these molecules transcytose across the BBB. In some embodiments, these molecules can bind directly or indirectly to human TMEM30A (UniProtKB—Q9NV96). In certain embodiments, these molecules bind better to cells that express TMEM30A than to cells that do not express (or which are modified to not express) TMEM30A. In some embodiments, these molecules have reduced lability in human serum than wild type FC5 (SEQ ID NO:1).

In some instances, the humanized FC5 polypeptide of this disclosure comprises an amino acid sequence that is at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%) identical to the sequence set forth in SEQ ID NO:1. In one instance, the humanized FC5 polypeptide comprises an amino acid sequence that is at least 85% identical to the sequence set forth in SEQ ID NO:1. These humanized FC5 polypeptides are able to transmigrate across the blood brain barrier and thus can ferry a “cargo” (e.g., a bio-active moiety such an antibody or antigen-binding fragment thereof, a small molecule drug, an antisense oligonucleotide, an enzyme, a peptide) to the CNS. In some embodiments, these molecules can bind directly or indirectly to human TMEM30A (UniProtKB—Q9NV96). In certain embodiments, these molecules bind better to cells that express TMEM30A than to cells that do not express (or which are modified to not express) TMEM30A. In some embodiments, these molecules have reduced lability in human serum than wild type FC5 (SEQ ID NO:1). In some embodiments, these FC5 polypeptides have amino acid substitutions, as compared to SEQ ID NO:1, at thirty or fewer (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) amino acids of SEQ ID NO:1. In certain embodiments, these FC5 polypeptides have amino acid substitutions, as compared to SEQ ID NO:1, at one or more (e.g., 1, 2, 3, 4, 5, 6, 7, 8, or 9) of the positions corresponding to positions 5, 6, 14, 75, 87, 88, 93, 114, and 117 of SEQ ID NO:1. The disclosure encompasses FC5 antibody variable domains with substitutions at all possible permutations of the above nine positions of SEQ ID NO:1. Non-limiting examples of the positions of SEQ ID NO:1 that may be substituted include: 5 and 6; 5, 6, and 14; 5, 6, 14, and 75; 5, 6, 14, 75, and 87; 5, 6, 14, 75, 87, and 88; 5, 6, 14, 75, 87, 88, and 93; 5, 6, 14, 75, 87, 88, 93, and 114; 5, 6, 14, 75, 87, 88, 93, 114, and 117; 5 and 14; 5, 14, and 75; 5, 14, 75, and 87; 5, 14, 75, 87, and 88; 5, 14, 75, 87, 88, and 93; 5, 14, 75, 87, 88, 93, and 114; 5, 14, 75, 87, 88, 93, 114, and 117; 5 and 75; 5, 75, and 87; 5, 75, 87, and 88; 5, 75, 87, 88, and 93; 5, 75, 87, 88, 93, and 114; 5, 75, 87, 88, 93, 114, and 117; 5 and 87; 5, 87, and 88; 5, 87, 88, and 93; 5, 87, 88, 93, and 114; 5, 87, 88, 93, 114, and 117; 5 and 88; 5, 88, and 93; 5, 88, 93, and 114; 5, 88, 93, 114, and 117; 5 and 93; 5, 93, and 114; 5, 93, 114, and 117; 5 and 114; 5, 114, and 117; and 5 and 117. In some embodiments, the above-described antibody variable domains further include a CDR1 comprising the sequence set forth in SEQ ID NO:47, a CDR2 comprising the sequence set forth in SEQ ID NO:50, and a CDR3 comprising the sequence set forth in SEQ ID NO:51. In other embodiments, these antibody variable domains further include a CDR1 comprising the sequence set forth in SEQ ID NO:57, a CDR2 comprising the sequence set forth in SEQ ID NO:50, and a CDR3 comprising the sequence set forth in SEQ ID NO:51. In some embodiments, the antibody variable domains comprises one or more (e.g., 1, 2, 3, 4, 5, 6, 7, 8, or 9) of: valine at the position corresponding to position 5 of SEQ ID NO:1; glutamic acid at the position corresponding to position 6 of SEQ ID NO:1; proline at the position corresponding to position 14 of SEQ ID NO:1; serine at the position corresponding to position 75 of SEQ ID NO:1; arginine at the position corresponding to position 87 of SEQ ID NO:1; alanine at the position corresponding to position 88 of SEQ ID NO:1; valine at the position corresponding to position 93 of SEQ ID NO:1; glutamine at the position corresponding to position 114 of SEQ ID NO:1; or leucine at the position corresponding to position 117 of SEQ ID NO:1. The above-described antibody variable domains may comprise further amino acid substitutions such as those described below.

In certain embodiments, the antibody variable domains have amino acid substitutions, as compared to SEQ ID NO:1, at one or more (e.g., 1, 2, 3, 4, 5, or 6) of the positions corresponding to positions 1, 37, 44, 45, 47, and 79 of SEQ ID NO:1. The disclosure encompasses antibody variable domains with substitutions at all possible permutations of the above six positions of SEQ ID NO:1.

In some instances, the above-described antibody variable domains further comprises amino acid substitutions, as compared to SEQ ID NO:1, at one or more of the positions corresponding to positions 44 and 45 of SEQ ID NO:1. In some cases, the amino acid sequence comprises glycine at the position corresponding to position 44 of SEQ ID NO:1 and/or leucine at the position corresponding to position 45 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In certain instances, the antibody variable domains of this disclosure further comprises amino acid substitutions, as compared to SEQ ID NO:1, at one or more of the positions corresponding to positions 1, 44 and 45 of SEQ ID NO:1. In some cases, the amino acid sequence comprises one or more (e.g., 1, 2, or 3) of: glutamic acid at the position corresponding to position 1 of SEQ ID NO:1; glycine at the position corresponding to position 44 of SEQ ID NO:1; or leucine at the position corresponding to position 45 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In other instances, the antibody variable domains of this disclosure further comprises amino acid substitutions, as compared to SEQ ID NO:1, at one or more of the positions corresponding to positions 44, 45, and 47 of SEQ ID NO:1. In some cases, the amino acid sequence comprises one or more (e.g., 1, 2, or 3) of: glycine at the position corresponding to position 44 of SEQ ID NO:1; leucine at the position corresponding to position 45 of SEQ ID NO:1; or tryptophan at the position corresponding to position 47 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In some instances, the antibody variable domains of this disclosure additionally comprises amino acid substitutions, as compared to SEQ ID NO:1, at one or more of the positions corresponding to positions 1, 44, 45, and 47 of SEQ ID NO:1. In some cases, the amino acid sequence comprises one or more (e.g., 1, 2, 3 or 4) of: glutamic acid at the position corresponding to position 1 of SEQ ID NO:1; glycine at the position corresponding to position 44 of SEQ ID NO:1; leucine at the position corresponding to position 45 of SEQ ID NO:1; or tryptophan at the position corresponding to position 47 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In certain instances, the antibody variable domains of this disclosure further comprises amino acid substitutions, as compared to SEQ ID NO:1, at one or more of the positions corresponding to positions 37, 44, 45, and 47 of SEQ ID NO:1. In some cases, the amino acid sequence comprises one or more (e.g., 1, 2, 3 or 4) of: valine at the position corresponding to position 37 of SEQ ID NO:1; glycine at the position corresponding to position 44 of SEQ ID NO:1; leucine at the position corresponding to position 45 of SEQ ID NO:1; or tryptophan at the position corresponding to position 47 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In some instances, the antibody variable domains of this disclosure additionally comprises amino acid substitutions, as compared to SEQ ID NO:1, at one or more of the positions corresponding to positions 1, 37, 44, 45, and 47 of SEQ ID NO:1. In some cases, the amino acid sequence comprises one or more (e.g., 1, 2, 3, 4, or 5) of: glutamic acid at the position corresponding to position 1 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; or tryptophan at the position corresponding to position 47 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In other instances, the antibody variable domains of this disclosure further comprises amino acid substitutions, as compared to SEQ ID NO:1, at one or more of the positions corresponding to positions 37, 44, 45, 47, and 79 of SEQ ID NO:1. In some cases, the amino acid sequence comprises one or more (e.g., 1, 2, 3, 4, or 5) of: valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; or leucine at the position corresponding to position 79 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

In some instances, the above-described antibody variable domains further comprises amino acid substitutions, as compared to SEQ ID NO:1, at one or more of the positions corresponding to positions 1, 37, 44, 45, 47, or 79 of SEQ ID NO:1. In some cases, the amino acid sequence comprises one or more (e.g., 1, 2, 3, 4, 5, or 6) of: glutamic acid at the position corresponding to position 1 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; or leucine at the position corresponding to position 79 of SEQ ID NO:1. These antibody variable domains are able to transmigrate across the blood brain barrier. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

Illustrative, non-limiting examples of humanized FC5 molecules are described in Example 4 of this disclosure. In certain instances, a humanized FC5 polypeptide comprises an amino acid sequence that is at least 80%, 85%, 90%, 91%, 92%, 93%, 94%, 95%, 96%, 97%, 98%, or 99% identical to an amino acid sequence set forth in any one of SEQ ID NOs.:10-17, wherein the humanized FC5 molecule can transcytose the BBB. In one embodiment, the humanized FC5 polypeptide comprises the amino acid sequence set forth in SEQ ID NO:10. In another embodiment, the humanized FC5 polypeptide comprises the amino acid sequence set forth in SEQ ID NO:11. In another embodiment, the humanized FC5 polypeptide comprises the amino acid sequence set forth in SEQ ID NO:12. In yet another embodiment, the humanized FC5 polypeptide comprises the amino acid sequence set forth in SEQ ID NO:13. In a different embodiment, the humanized FC5 polypeptide comprises the amino acid sequence set forth in SEQ ID NO:14. In another embodiment, the humanized FC5 polypeptide comprises the amino acid sequence set forth in SEQ ID NO:15. In yet another embodiment, the humanized FC5 polypeptide comprises the amino acid sequence set forth in SEQ ID NO:16. In some embodiments, the humanized FC5 polypeptide comprises the amino acid sequence set forth in SEQ ID NO:17. The biological activity of humanized FC5 variants can be assessed using, e.g., the in vitro BBB transwell cell culture assay using SV-ARBEC cells as described in Example 2 of this disclosure.

### Stabilization of FC5

As shown in Example 3 of the present disclosure, FC5 can be labile in serum. Proteolysis may occur at one or more sites within FC5 when exposed to serum. Stabilizing FC5 can reduce its susceptibility for proteolytic degradation so that the FC5 can be developed for clinical use. In one embodiment, the FC5 molecules of this disclosure are stabilized by introducing a second disulfide bond within the FC5 molecule.

FC5 isolated from a naive llama single-domain antibody library contains a single disulfide bond. About half of the VHH germlines of all camelid species contain a third cysteine, thus providing for the acquisition of a fourth cysteine during somatic hypermutation, and many camelid VHHs have two disulfides.

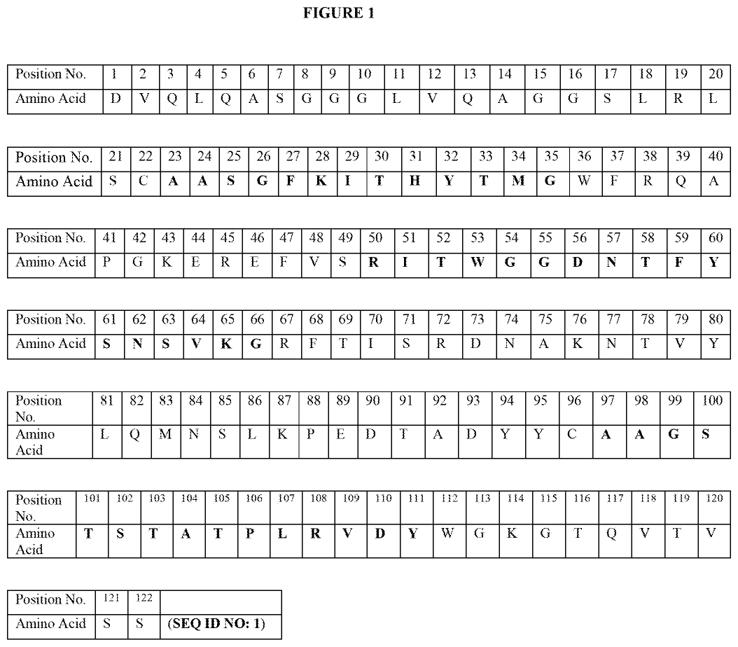

Several strategies were pursued to add a second disulfide bond to FCS, in addition to the cysteine formed by Cys22 and Cys96 that is common to all immunoglobulin variable domains. In a first strategy, FC5 was engineered to include a second disulfide formed by cysteines at positions 34 and 79 (the position numbering is based on FIG. 1), connecting β-strands C and E within framework regions 2 and 3, respectively. C34 is at the end of CDR-H1, at the beginning of β-strand C. This disulfide is located in the interior of the protein globule and can improve thermal melting temperatures (Tm) between 4 and 18° C. A second strategy involved combining spatially close interior positions that can accommodate disulfide-forming cysteines. This strategy relies on positions 49 and 70 (the position numbering is based on FIG. 1), connecting strands C′ and E, again in framework regions 2 and 3. For this strategy, Tm can be improved between 6 and 19° C. A third disulfide stabilization strategy was to connect exterior-facing position 33 in CDR-H1 with various positions in CDR-H3 (positions 102, 103, 104, and 105), which in VHH is often folded over the flank corresponding to the VH/VL interface in conventional antibodies. Therefore, this interloop disulfide bond is partially solvent-exposed.

The disclosure features an antibody variable domain that transmigrates across the blood brain barrier and that is at least 80% identical (e.g., at least 85%, at least 86%, at least 87%, at least 88%, at least 89%, at least 90%, at least 91%, at least 92%, at least 95%, at least 97%, or 100% identical) to SEQ ID NO:1. In some instances, the antibody variable domain comprises a CDR1 comprising the sequence set forth in SEQ ID NO:47, CDR2 comprising the sequence set forth in SEQ ID NO:50, CDR3 comprising the sequence set forth in SEQ ID NO:51, and the amino acid sequence comprises cysteines at the positions corresponding to positions 49 and 70 of SEQ ID NO:1. In other instances, the antibody variable domain comprises a CDR1 comprising the sequence set forth in SEQ ID NO:48, CDR2 comprising the sequence set forth in SEQ ID NO:50, CDR3 comprising the sequence set forth in SEQ ID NO:51, and the amino acid sequence comprises cysteine at the position corresponding to position 79 of SEQ ID NO:1. In certain instances, the antibody variable domain comprises a CDR1 comprising the sequence set forth in SEQ ID NO:49, CDR2 comprising the sequence set forth in SEQ ID NO:50, and CDR3 comprises the sequence set forth in SEQ ID NO:52. In some instances, the antibody variable domain comprises a CDR1 comprises the sequence set forth in SEQ ID NO:58, CDR2 comprising the sequence set forth in SEQ ID NO:50, and CDR3 comprises the sequence set forth in SEQ ID NO:52. In certain instances, the antibody variable domain comprises a CDR1 comprising the sequence set forth in SEQ ID NO:49, CDR2 comprising the sequence set forth in SEQ ID NO:50, and CDR3 comprising the sequence set forth in SEQ ID NO:53. In certain instances, the antibody variable domain comprises a CDR1 comprising the sequence set forth in SEQ ID NO:58, CDR2 comprising the sequence set forth in SEQ ID NO:50, and CDR3 comprises the sequence set forth in SEQ ID NO:53. In some instances, the antibody variable domain comprises a CDR1 comprises the sequence set forth in SEQ ID NO:49, CDR2 comprising the sequence set forth in SEQ ID NO:50, and CDR3 comprises the sequence set forth in SEQ ID NO:54. In certain instances, the antibody variable domain comprises a CDR1 comprising the sequence set forth in SEQ ID NO:58, CDR2 comprising the sequence set forth in SEQ ID NO:50, and CDR3 comprising the sequence set forth in SEQ ID NO:54. In certain instances, the antibody variable domain comprises a CDR1 comprising the sequence set forth in SEQ ID NO:49, CDR2 comprising the sequence set forth in SEQ ID NO:50, and CDR3 comprising the sequence set forth in SEQ ID NO:55. In yet another case, the antibody variable domain comprises a CDR1 comprising the sequence set forth in SEQ ID NO:58, CDR2 comprising the sequence set forth in SEQ ID NO:50, and CDR3 comprising the sequence set forth in SEQ ID NO:55. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

The above-described antibody variable domains may include thirty or fewer (e.g., 30, 29, 28, 27, 26, 25, 24, 23, 22, 21, 20, 19, 18, 17, 16, 15, 14, 13, 12, 10, 9, 8, 7, 6, 5, 4, 3, 2, or 1) amino acid substitutions relative to the amino acid sequence set forth in SEQ ID NO:1. In certain cases, the amino acid substitutions are conservative amino acid substitutions. In some instances, the antibody variable domain comprises, as compared to SEQ ID NO:1, substitutions at one or more (e.g., 1, 2, 3, 4, 5, 6, 7, 8, 9, 10, 11, 12, 13, 14) of the positions corresponding to positions 1, 5, 6, 14, 37, 44, 45, 47, 75, 87, 88, 93, 114, or 117 of SEQ ID NO:1. In certain instances, the amino acid sequence comprises one or more (e.g., 1, 2, 3, 4, 5, 6, 7, 8, 9, 10, 11, 12, 13, 14) of: glutamic acid at the position corresponding to position 1 of SEQ ID NO:1; valine at the position corresponding to position 5 of SEQ ID NO:1; glutamic acid at the position corresponding to position 6 of SEQ ID NO:1; proline at the position corresponding to position 14 of SEQ ID NO:1; valine at the position corresponding to position 37 of SEQ ID NO:1; glycine at the position corresponding to position 44 of SEQ ID NO:1; leucine at the position corresponding to position 45 of SEQ ID NO:1; tryptophan at the position corresponding to position 47 of SEQ ID NO:1; serine at the position corresponding to position 75 of SEQ ID NO:1; arginine at the position corresponding to position 87 of SEQ ID NO:1; alanine at the position corresponding to position 88 of SEQ ID NO:1; valine at the position corresponding to position 93 of SEQ ID NO:1; glutamine at the position corresponding to position 114 of SEQ ID NO:1; or leucine at the position corresponding to position 117 of SEQ ID NO:1. In some embodiments, these molecules can bind directly or indirectly to human TMEM30A (UniProtKB—Q9NV96). In certain embodiments, these molecules bind better to cells that express TMEM30A than to cells that do not express or which are modified to not express TMEM30A. In some embodiments, these molecules have reduced lability in human serum than wild type FC5 (SEQ ID NO:1).

The disulfide stabilization strategy may be employed with wild type FC5 or a humanized FC5 molecule. Example 5 of this disclosure provides non-limiting examples of disulfide stabilized antibody variable domains. This disclosure encompasses an antibody variable domain comprising an amino acid sequence that is at least 80%, 85%, 90%, 91%, 92%, 93%, 94%, 95%, 96%, 97%, 98%, 99% identical to any one of SEQ ID NOs.:20-28, wherein the antibody variable domain can transcytose the BBB. In one embodiment, the disulfide stabilized antibody variable domain has an amino acid sequence set forth in SEQ ID NO:20. In another embodiment, the disulfide stabilized antibody variable domain has an amino acid sequence set forth in SEQ ID NO:21. In another embodiment, the disulfide stabilized antibody variable domain has an amino acid sequence set forth in SEQ ID NO:22. In yet another embodiment, the disulfide stabilized antibody variable domain has an amino acid sequence set forth in SEQ ID NO:23. In another embodiment, the disulfide stabilized antibody variable domain has an amino acid sequence set forth in SEQ ID NO:24. In some embodiments, the disulfide stabilized antibody variable domain has an amino acid sequence set forth in SEQ ID NO:25. In another embodiment, the disulfide stabilized antibody variable domain has an amino acid sequence set forth in SEQ ID NO:26. In another embodiment, the disulfide stabilized antibody variable domain has an amino acid sequence set forth in SEQ ID NO:27. In certain embodiments, the disulfide stabilized antibody variable domain has an amino acid sequence set forth in SEQ ID NO:28.

### Chimeric Molecules

This disclosure also features chimeric molecules comprising any of the antibody variable domains disclosed herein (e.g., humanized, disulfide-stabilized, or humanized and disulfide-stabilized antibody variable domains). In certain embodiments, the chimeric molecule comprises a dimeric moiety (e.g., Fc) or other fusion protein (e.g. human serum albumin (HSA)—it is noted that albumin is not dimeric, but can be fused at its N- and/or C-terminus). In some instances, the C-terminal of an antibody variable domain disclosed herein is directly or indirectly linked to the N-terminal of each member of the dimeric moiety. In certain cases, the chimeric molecule comprises an antibody, an antigen-binding fragment of an antibody, a single chain antibody, a peptide, an enzyme, a nucleic acid (e.g., an antisense oligonucleotide), a small molecule drug, or a liposome or a lipid nanoparticle encapsulating a bio-active moiety such as a nucleic acid, small molecule drug, or peptide. Non-limiting examples of chimeric fusions of this disclosure are provided in schematic form in FIG. 48.

### Fc Region

In some instances, the chimeric molecule includes an antibody Fc region. As used herein, the term “Fc region” is defined as the portion of an immunoglobulin formed by the dimeric association of the respective Fc domains (or Fc moieties) of its two heavy chains. A native Fc region is homodimeric and comprises two polypeptide chains. The Fc regions of this disclosure can be a homodimer (identical Fc domains) or a heterodimer (non-identical Fc domains). As used herein, the term “Fc domain” or “Fc moiety” refers to a single polypeptide chain of an immunoglobulin comprising a CH2 domain and a CH3 domain, or a variant thereof. In some embodiments, the Fc region is from human IgG1, human IgG2, human IgG3, or human IgG4. In certain embodiments, the Fc region is aglycosylated (e.g., N297Q, or T299A). In certain embodiments, the Fc region is a human IgG1 agly Fc region. In certain embodiments, the Fc region contains a human IgG4P agly CH2 domain and a human IgG1 CH3 domain. In certain instances, the Fc region or Fc domain has reduced effector function relative to its wild type counterpart. IgG antibodies exist in various allotypes and isoallotypes and each of these are encompassed by the present disclosure. In certain embodiments, an Fc region of the present disclosure includes an IgG1 heavy chain Fc having an allotype of G1m1; nG1m2; G1m3; G1m17,1; G1m17,1,2; G1m3,1; or G1m17. These allotypes or isoallotypes are characterized by the following amino acid residues at the indicated positions within the IgG1 heavy chain constant region (Fc) (EU numbering): G1m1: D356, L358; nG1m1: E356, M358; G1m3: R214, E356, M358, A431; G1m17,1: K214, D356, L358, A431; G1m17,1,2: K214, D356, L358, G431; G1m3,1: R214, D356, L358, A431; and G1m17: K214, E356, M358, A431. Exemplary Fc domain sequences are provided below:

Fc of IgG1 (IgG1m3):
(SEQ ID NO: 61)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPG
Fc of IgG1 agly (it is to be understood that
other agly versions such as T299A can be
employed):
(SEQ ID NO: 62)
APELLGGPSVFLFPPKPKDTLMISRTPEVTCVVVDVSHEDPEVKFNWYV
DGVEVHNAKTKPREEQYQSTYRVVSVLTVLHQDWLNGKEYKCKVSNKAL
PAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPG
Fc of IgG2:
(SEQ ID NO: 63)
APPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSV
MHEALHNHYTQKSLSLSPG
Fc of IgG2 SAA:
(SEQ ID NO: 64)
APPAAAPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSV
MHEALHNHYTQKSLSLSPG

It is noted that the “SAA” version also includes a C222S mutation in the hinge:

(SEQ ID NO: 95)
ERKSCVECPPCP
Fc of IgG3:
(SEQ ID NO: 65)
APELLGGPSVFLFPPKPKDTLMISRTPEVTCVVVDVSHEDPEVQFKWYV
DGVEVHNAKTKPREEQYNSTFRVVSVLTVLHQDWLNGKEYKCKVSNKAL
PAPIEKTISKTKGQPREPQVYTLPPSREEMTKNQVSLTCLVKGFYPSDI
AVEWESSGQPENNYNTTPPMLDSDGSFFLYSKLTVDKSRWQQGNIFSCS
VMHEALHNRFTQKSLSLSPG
Fc of IgG4:
(SEQ ID NO: 66)
APEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCS
VMHEALHNHYTQKSLSLSLG
Fc of IgG4 agly/G1:
(SEQ ID NO: 67)
APEFLGGPSVFLFPPKPKDTLMISRTPEVTCVVVDVSQEDPEVQFNWYV
DGVEVHNAKTKPREEQFQSTYRVVSVLTVLHQDWLNGKEYKCKVSNKGL
PSSIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCS
VMHEALHNHYTQKSLSLSPG

In certain instances, the Fc domains may include one or mutations relative to the wild type Fc domain. For example, the one or more mutations may be made to: lower effector function, increase stability, improve pharmacokinetics, and/or increase half-life. In certain cases, the very C-terminal residue of each IgG1 Fc domain is deleted (i.e., the final lysine is removed) because there can be splicing and/or enzymatic cleavage at this C-terminal lysine which can lead to product heterogeneity. In certain instances, the very C-terminal residue of the IgG1 Fc domain is a lysine residue. In certain instances, the very C-terminal residue of the IgG1 Fc domain is changed from a lysine to a cysteine residue. This allows for C-terminal site conjugation. In some instances, the Fc domain comprises the S442C mutation (numbering according to EU index). In some cases, the mutation(s) may be introduced to promote heterodimerization (e.g., comprising knob-into-hole mutations, electrosteering mutations, DuoBody mutations, etc.). Such mutations are well known in the art. See, e.g., Ridgway et al., Protein Engineering 9(7):617-21 (1996); U.S. Pat. Nos. 5,807,706; 9,862,778; WO2017/106462; Labrijn et al, PNAS, 110(13):5145-5150 (2013); Gramer et al. mAbs, 5(6): 962-973 (2013); Labrijn et al. Nature Protocols, 9(10):2450-63 (2014).
Fc Domains Comprising Knob into Hole Mutations:

(SEQ ID NO: 68)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLSCAVKGFYPSDI
AVEWESNGQPENNYKTTPPVLDSDGSFFLVSKLTVDKSRWQQGNVFSCS
VMHEALHNHYTQKSLSLSPG
(SEQ ID NO: 69)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLWCLVKGFYPSDI
AVEWESNGQPENNYKTTPPVLDSDGSFFLYSKLTVDKSRWQQGNVFSCS
VMHEALHNHYTQKSLSLSPG

### Fc Domains Comprising Electrosteering Mutations:

(SEQ ID NO: 70)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVEWESNGQPENNYKTTPPVLDSDGSFFLYSDLTVDKSRWQQGNVFSCS
VMHEALHNHYTQKSLSLSPG
(SEQ ID NO: 71)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVEWESNGQPENNYKTTPPVLKSDGSFFLYSKLTVDKSRWQQGNVFSCS
VMHEALHNHYTQKSLSLSPG

### Fc Domains Comprising DuoBody Mutations:

### Pair 1:

Kuhlman Demarest 20.8.34: ″a″ side
(SEQ ID NO: 72)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVSTLPPSRDELTKNQVSLMCLVYGFYPSDI
AVEWESNGQPENNYKTTPPVLDSDGSFFLYSVLTVDKSRWQQGNVFSCS
VMHEALHNHYTQKSLSLSPG
Kuhlman Demarest 20.8.34: ″b″ side
(SEQ ID NO: 73)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRGDLTKNQVQLTCLVKGFYPSDI
AVEWESNGQPENNYKTTPPVLDSDGSFFLASKLTVDKSRWQQGNVFSCS
VMHEALHNHYTQKSLSLSPG

### Pair 2:

Kuhlman Demarest 7.8.60: ″a″ side
(SEQ ID NO: 96)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVDGFYPSDI
AVEWESNGQPENNYKTTPPVLMSDGSFFLASKLTVDKSRWQQGNVFSCS
VMHEALHNHYTQKSLSLSPG
Kuhlman Demarest 7.8.60: ″b″ side
(SEQ ID NO: 97)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPRRPRVYTLPPSRDELTKNQVSLVCLVKGFYPSDI
AVEWESNGQPENNYKTTPPVLDSDGSFFLYSVLTVDKSRWQQGNVFSCS
VMHEALHNHYTQKSLSLSPG

### Pair 3:

MP43a: S364K/K409L
(SEQ ID NO: 98)
APELLGGPSVFLFPPKPKDTLMISRTPEVTCVVVDVSHEDP
EVKFNWYVDGVEVHNAKTKPREEQYNSTYRWSVLTVLHQDW
LNGKEYKCKVSNKALPAPIEKTISKAKGQPREPQVYTLPPS
RDELTKNQVKLTCLVKGFYPSDIAVEWESNGQPENNYKTTP
PVLDSDGSFFLYSLLTVDKSRWQQGNVFSCSVMHEALHNHY
TQKSLSLSPG
MP43b: K370S/F405K
(SEQ ID NO: 99)
APELLGGPSVFLFPPKPKDTLMISRTPEVTCVVVDVSHED
PEVKFNWYVDGVEVHNAKTKPREEQYNSTYRWSVLTVLHQ
DWLNGKEYKCKVSNKALPAPIEKTISKAKGQPREPQVYTL
PPSRDELTKNQVSLTCLVSGFYPSDIAVEWESNGQPENNY
KTTPPVLDSDGSFKLYSKLTVDKSRWQQGNVFSCSVMHEA
LHNHYTQKSLSLSPG

### Pair 4:

ZW1a: T350V/L351Y/F405A/Y407V
(SEQ ID NO: 100)
APELLGGPSVFLFPPKPKDTLMISRTPEVTCVVVDVSHED
PEVKFNWYVDGVEVHNAKTKPREEQYNSTYRWSVLTVLHQ
DWLNGKEYKCKVSNKALPAPIEKTISKAKGQPREPQVYVY
PPSRDELTKNQVSLTCLVKGFYPSDIAVEWESNGQPENNY
KTTPPVLDSDGSFALVSKLTVDKSRWQQGNVFSCSVMHEA
LHNHYTQKSLSLSPG
ZW1b: T350V/T366L/K392L/T394W
(SEQ ID NO: 101)
APELLGGPSVFLFPPKPKDTLMISRTPEVTCVWDVSHEDP
EVKFNWYVDGVEVHNAKTKPREEQYNSTYRVVSVLTVLHQ
DWLNGKEYKCKVSNKALPAPIEKTISKAKGQPREPQVYVL
PPSRDELTKNQVSLLCLVKGFYPSDIAVEWESNGQPENNY
LTWPPVLDSDGSFFLYSKLTVDKSRWQQGNVFSCSVMHEA
LHNHYTQKSLSLSPG

In certain instances, an Fc region has Fc domains that are at least 80%, 85%, 90%, 91%, 92%, 93%, 94%, 95%, 96%, 97%, 98%, 99% identical to any one of SEQ ID NOs.:61-101. In certain embodiments, the Fc region has lower effector function than the corresponding wild type Fc region.

### Hinge Region

In certain instances, the chimeric molecule can include an antibody hinge region. In some instances, the chimeric molecule includes an antibody hinge region and an antibody Fc region. The hinge region can be fused directly or via a linker to an Fc moiety. For example, the hinge connects the C-terminus of an antibody variable domain described above and the N-terminus of a CH2 domain of an Fc domain. The hinge region can be any flexible peptide sequence of one to 30 amino acids (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) in length. In some instances, the hinge region is one to 20 amino acids in length. In other instances, the hinge region is one to 18 amino acids in length. In some cases, the hinge region is the hinge from human IgG1, human IgG2, human IgG3, or human IgG4. In certain cases, these hinge regions may be engineered to include one or more amino acid substitutions (e.g., S228P in IgG4). In certain instances, the hinge region comprises the amino acid sequence AEPKSCD (SEQ ID NO:56). In other instances, the hinge region comprises the amino acid sequence AEPKSSD (SEQ ID NO:59). In yet other instances, the hinge region comprises the amino acid sequence KTHTCPPCP (SEQ ID NO:19). In certain instances, the hinge region comprises the amino acid sequence AEPKSCDKTHTCPPCP (SEQ ID NO:74). In other instances, the hinge region comprises the amino acid sequence AEPKSSDKTHTCPPCP (SEQ ID NO:75). In other instances, the hinge region comprises the amino acid sequence GGGGSDKTHTCPPCP (SEQ ID NO:76). In some instances, the hinge region comprises the amino acid sequence VERKCCVECPPCP (SEQ ID NO:102). In other instances, the hinge region comprises the amino acid sequence VESKYGPPCPSCP (SEQ ID NO:103). In other instances, the hinge region comprises the amino acid sequence ELKTPLGDTTHTCPRCP (SEQ ID NO:104). In yet other instances, the hinge region comprises the amino acid sequence EPKSCDTPPPCPRCP (SEQ ID NO:105).

In certain embodiments, the chimeric molecule homo- or hetero-dimerizes with another chimeric molecule. For example, if the Fc domains of the chimeric molecule are identical they homodimerize to form a homodimeric Fc region. If however, the Fc domains of the chimeric molecule are not identical (e.g., they contain knob-into-hole, electrosteering or DuoBody mutations) they heterodimerize to form a heterodimeric Fc region. The C-terminus of the antibody variable domains of the disclosure can be linked directly or via an intervening amino acid sequence to the N-terminus of a hinge region which in turn is linked via its C-terminus to the N-terminus of a CH2 domain of an Fc domain.

### Linkers

In some instances, the chimeric molecule includes one or more linkers. There is no particular limitation on the linkers that link different regions of the chimeric molecule (e.g., link the antibody variable domain to the N-terminus of the VH or VL domain of a therapeutic antibody; link the antibody variable domain to a therapeutic moiety; link the C-terminus of an Fc domain of an Fc region to the N-terminus of a therapeutic antibody or fragment thereof; link the C-terminus of an Fc domain of an Fc region to a therapeutic moiety; link the antibody variable domain to a hinge region). In some embodiments, the linker is a peptide linker. Any arbitrary single-chain peptide comprising about one to 30 amino acid residues (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 aa residues) can be used as a linker. Examples of such peptide linkers include: Gly; Ser; Gly Ser; Gly Gly Ser; Ser Gly Gly; Gly Gly Gly Ser (SEQ ID NO:77); Ser Gly Gly Gly (SEQ ID NO:78); Gly Gly Gly Gly Ser (SEQ ID NO:5); Ser Gly Gly Gly Gly (SEQ ID NO:79); Gly Gly Gly Gly Gly Ser (SEQ ID NO:80); Ser Gly Gly Gly Gly Gly (SEQ ID NO:81); Gly Gly Gly Gly Gly Gly Ser (SEQ ID NO:82); Ser Gly Gly Gly Gly Gly Gly (SEQ ID NO:83); (Gly Gly Gly Gly Ser)ₙ(SEQ ID NO:5)ₙ, wherein n is an integer of one or more; and (Ser Gly Gly Gly Gly)ₙ(SEQ ID NO:79)ₙ, wherein n is an integer of one or more.

In other embodiments, the linker peptides are modified such that the amino acid sequence GSG (that occurs at the junction of traditional Gly/Ser linker peptide repeats) is not present. For example, the peptide linker comprise an amino acid sequence selected from the group consisting of: (GGGXX)ₙGGGGS (SEQ ID NO:84) and GGGGS(XGGGS)ₙ(SEQ ID NO:85), where X is any amino acid that can be inserted into the sequence and not result in a polypeptide comprising the sequence GSG, and n is 0 to 4. In one embodiment, the sequence of a linker peptide is (GGGX₁X₂)ₙGGGGS and X₁ is P and X₂ is S and n is 0 to 4 (SEQ ID NO:86). In another embodiment, the sequence of a linker peptide is (GGGX₁X₂)ₙGGGGS and X₁ is G and X₂ is Q and n is 0 to 4 (SEQ ID NO:87). In another embodiment, the sequence of a linker peptide is (GGGX₁X₂)ₙGGGGS and X₁ is G and X₂ is A and n is 0 to 4 (SEQ ID NO:88). In yet another embodiment, the sequence of a linker peptide is GGGGS(XGGGS)ₙ, and X is P and n is 0 to 4 (SEQ ID NO:89). In one embodiment, a linker peptide of the invention comprises or consists of the amino acid sequence (GGGGA)2GGGGS (SEQ ID NO:90). In another embodiment, a linker peptide comprises or consists of the amino acid sequence (GGGGQ)2GGGGS (SEQ ID NO:91). In yet another embodiment, a linker peptide comprises or consists of the amino acid sequence (GGGPS)2GGGGS (SEQ ID NO:92). In a further embodiment, a linker peptide comprises or consists of the amino acid sequence GGGGS(PGGGS)2 (SEQ ID NO:93).

In certain embodiments, the linker is a synthetic compound linker (chemical cross-linking agent). Examples of cross-linking agents include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), bis[2-(sulfosuccinimidooxycarbonyloxy)ethyl]sulfone (sulfo-BSOCOES), sulfo-SMCC series and bismaleimidohexane (BMH) series crosslinkers.

### XTEN

In some instances, the chimeric molecules include an XTEN sequence. “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a chimeric molecule partner, XTENs can serve as a carrier, conferring certain desirable pharmacokinetic, physicochemical and pharmaceutical properties when linked to a clotting factor, a heavy chain of a clotting factor, a light chain or a clotting factor, a targeting moiety, or any other sequences or molecules on the chimeric molecule. Such desirable properties include but are not limited to enhanced pharmacokinetic parameters and solubility characteristics.

In some embodiments, the XTEN sequence of the invention is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues.

The table below provides the amino acid sequences of non-limiting XTENs.

### Exemplary XTEN Sequences

| XTEN | Amino Acid Sequence |
|---|---|
| AE42 | GAPGSPAGSPTSTEEGTSES |
| SEQ ID NO: 106 | ATPESGPGSEPATSGSETPA |
| | SS |
| AE42_2 | TGGGSPAGSPTSTEEGTSES |
| SEQ ID NO: 107 | ATPESGPGSEPATSGSETPA |
| | SS |
| AE42_3 | GTSESATPESGPGSEPATSG |
| SEQ ID NO: 108 | SETPGTSESATPESGPGSEP |
| | AT |
| AE72 | GAPTSESATPESGPGSEPAT |
| SEQ ID NO: 109 | SGSETPGTSESATPESGPGS |
| | EPATSGSETPGTSESATPES |
| | GPGTSTEPSEGSAPGASS |
| AE72_2 | GTSESATPESGPGSEPATSG |
| SEQ ID NO: 110 | SETPGTSESATPESGPGSEP |
| | ATSGSETPGTSESATPESGP |
| | GTSTEPSEGSAP |
| AE72_3 | SPAGSPTSTEEGTSESATPE |
| SEQ ID NO: 111 | SGPGSEPATSGSETPGTSES |
| | ATPESGPGTSTEPSEGSAPG |
| | TSTEPSEGSAPG |
| AE144 | GSEPATSGSETPGTSESATP |
| SEQ ID NO: 112 | ESGPGSEPATSGSETPGSPA |
| | GSPTSTEEGTSTEPSEGSAP |
| | GSEPATSGSETPGSEPATSG |
| | SETPGSEPATSGSETPGTST |
| | EPSEGSAPGTSESAPESGPG |
| | SEPATSGSETPGTSTEPSEG |
| | SAP |
| AE144_2 | GTSESATPESGPGSEPATSG |
| SEQ ID NO: 113 | SETPGTSESATPESGPGSEP |
| | ATSGSETPGTSESATPESGP |
| | GTSTEPSEGSAPGSPAGSPT |
| | STEEGTSESATPESGPGSEP |
| | ATSGSETPGTSESATPESGP |
| | GSPAGSPTSTEEGSPAGSPT |
| | STEE |
| AE144_3 | GSPAGSPTSTEEGTSESATP |
| SEQ ID NO: 114 | ESGPGTSTEPSEGSAPGSPA |
| | GSPTSTEEGTSTEPSEGSAP |
| | GTSTEPSEGSAPGTSESATP |
| | ESGPGSEPATSGSETPGSEP |
| | ATSGSETPGSPAGSPTSTEE |
| | GTSESATPESGPGTSTEPSE |
| | GSAP |
| AG144 | GTPGSGTASSSPGSSTPSGA |
| SEQ ID NO: 115 | TGSPGSSPSASTGTGPGSSP |
| | SASTGTGPGASPGTSSTGSP |
| | GASPGTSSTGSPGSSTPSGA |
| | TGSPGSSPSASTGTGPGASP |
| | GTSSTGSPGSSPSASTGTGP |
| | GTPGSGTASSSPGSSTPSGA |
| | TGSP |
| AE288 | GTSESATPESGPGSEPATSG |
| SEQ ID NO: 116 | SETPGTSESATPESGPGSEP |
| | ATSGSETPGTSESATPESGP |
| | GTSTEPSEGSAPGSPAGSPT |
| | STEEGTSESATPESGPGSEP |
| | ATSGSETPGTSESATPESGP |
| | GSPAGSPTSTEEGSPAGSPT |
| | STEEGTSTEPSEGSAPGTSE |
| | SATPESGPGTSESATPESGP |
| | GTSESATPESGPGSEPATSG |
| | SETPGSEPATSGSETPGSPA |
| | GSPTSTEEGTSTEPSEGSAP |
| | GTSTEPSEGSAPGSEPATSG |
| | SETPGTSESATPESGPGTST |
| | EPSEGSAP |
| AG288 | PGASPGTSSTGSPGASPGTS |
| SEQ ID NO: 117 | STGSPGTPGSGTASSSPGSS |
| | TPSGATGSPGTPGSGTASSS |
| | PGSSTPSGATGSPGTPGSGT |
| | ASSSPGSSTPSGATGSPGSS |
| | TPSGATGSPGSSPSASTGTG |
| | PGSSPSASTGTGPGASPGTS |
| | STGSPGTPGSGTASSSPGSS |
| | TPSGATGSPGSSPSASTGTG |
| | PGSSPSASTGTGPGASPGTS |
| | STGSPGASPGTSSTGSPGSS |
| | TPSGATGSPGSSPSASTGTG |
| | PGASPGTSSTGSPGSSPSAS |
| | TGTGPGTPGSGTASSSPGSS |
| | TPSGATGS |
| AES76 | GSPAGSPTSTEEGTSESATP |
| SEQ ID NO: 118 | ESGPGTSTEPSEGSAPGSPA |
| | GSPTSTEEGTSTEPSEGSAP |
| | GTSTEPSEGSAPGTSESATP |
| | ESGPGSEPATSGSETPGSEP |
| | ATSGSETPGSPAGSPTSTEE |
| | GTSESATPESGPGTSTEPSE |
| | GSAPGTSTEPSEGSAPGSPA |
| | GSPTSTEEGTSTEPSEGSAP |
| | GTSTEPSEGSAPGTSESATP |
| | ESGPGTSTEPSEGSAPGTSE |
| | SATPESGPGSEPATSGSETP |
| | GTSTEPSEGSAPGTSTEPSE |
| | GSAPGTSESATPESGPGTSE |
| | SATPESGPGSPAGSPTSTEE |
| | GTSESATPESGPGSEPATSG |
| | SETPGTSESATPESGPGTST |
| | EPSEGSAPGTSTEPSEGSAP |
| | GTSTEPSEGSAPGTSTEPSE |
| | GSAPGTSTEPSEGSAPGTST |
| | EPSEGSAPGSPAGSPTSTEE |
| | GTSTEPSEGSAPGTSESATP |
| | ESGPGSEPATSGSETPGTSE |
| | SATPESGPGSEPATSGSETP |
| | GTSESATPESGPGTSTEPSE |
| | GSAPGTSESATPESGPGSPA |
| | GSPTSTEEGSPAGSPTSTEE |
| | GSPAGSPTSTEEGTSESATP |
| | ESGPGTSTEPSEGSAP |
| AGS76 | PGTPGSGTASSSPGSSTPSG |
| SEQ ID NO: 119 | ATGSPGSSPSASTGTGPGSS |
| | PSASTGTGPGSSTPSGATGS |
| | PGSSTPSGATGSPGASPGTS |
| | STGSPGASPGTSSTGSPGAS |
| | PGTSSTGSPGTPGSGTASSS |
| | PGASPGTSSTGSPGASPGTS |
| | STGSPGASPGTSSTGSPGSS |
| | PSASTGTGPGTPGSGTASSS |
| | PGASPGTSSTGSPGASPGTS |
| | STGSPGASPGTSSTGSPGSS |
| | TPSGATGSPGSSTPSGATGS |
| | PGASPGTSSTGSPGTPGSGT |
| | ASSSPGSSTPSGATGSPGSS |
| | TPSGATGSPGSSTPSGATGS |
| | PGSSPSASTGTGPGASPGTS |
| | STGSPGASPGTSSTGSPGTP |
| | GSGTASSSPGASPGTSSTGS |
| | PGASPGTSSTGSPGASPGTS |
| | STGSPGASPGTSSTGSPGTP |
| | GSGTASSSPGSSTPSGATGS |
| | PGTPGSGTASSSPGSSTPSG |
| | ATGSPGTPGSGTASSSPGSS |
| | TPSGATGSPGSSTPSGATGS |
| | PGSSPSASTGTGPGSSPSAS |
| | TGTGPGASPGTSSTGSPGTP |
| | GSGTASSSPGSSTPSGATGS |
| | PGSSPSASTGTGPGSSPSAS |
| | TGTGPGASPGTSSTGS |
| AE864 | GSPAGSPTSTEEGTSESATP |
| SEQ ID NO: 120 | ESGPGTSTEPSEGSAPGSPA |
| | GSPTSTEEGTSTEPSEGSAP |
| | GTSTEPSEGSAPGTSESATP |
| | ESGPGSEPATSGSETPGSEP |
| | ATSGSETPGSPAGSPTSTEE |
| | GTSESATPESGPGTSTEPSE |
| | GSAPGTSTEPSEGSAPGSPA |
| | GSPTSTEEGTSTEPSEGSAP |
| | GTSTEPSEGSAPGTSESATP |
| | ESGPGTSTEPSEGSAPGTSE |
| | SATPESGPGSEPATSGSETP |
| | GTSTEPSEGSAPGTSTEPSE |
| | GSAPGTSESATPESGPGTSE |
| | SATPESGPGSPAGSPTSTEE |
| | GTSESATPESGPGSEPATSG |
| | SETPGTSESATPESGPGTST |
| | EPSEGSAPGTSTEPSEGSAP |
| | GTSTEPSEGSAPGTSTEPSE |
| | GSAPGTSTEPSEGSAPGTST |
| | EPSEGSAPGSPAGSPTSTEE |
| | GTSTEPSEGSAPGTSESATP |
| | ESGPGSEPATSGSETPGTSE |
| | SATPESGPGSEPATSGSETP |
| | GTSESATPESGPGTSTEPSE |
| | GSAPGTSESATPESGPGSPA |
| | GSPTSTEEGSPAGSPTSTEE |
| | GSPAGSPTSTEEGTSESATP |
| | ESGPGTSTEPSEGSAPGTSE |
| | SATPESGPGSEPATSGSETP |
| | GTSESATPESGPGSEPATSG |
| | SETPGTSESATPESGPGTST |
| | EPSEGSAPGSPAGSPTSTEE |
| | GTSESATPESGPGSEPATSG |
| | SETPGTSESATPESGPGSPA |
| | GSPTSTEEGSPAGSPTSTEE |
| | GTSTEPSEGSAPGTSESATP |
| | ESGPGTSESATPESGPGTSE |
| | SATPESGPGSEPATSGSETP |
| | GSEPATSGSETPGSPAGSPT |
| | STEEGTSTEPSEGSAPGTST |
| | EPSEGSAPGSEPATSGSETP |
| | GTSESATPESGPGTSTEPSE |
| | GSAP |
| AG864 | GASPGTSSTGSPGSSPSAST |
| SEQ ID NO: 121 | GTGPGSSPSASTGTGPGTPG |
| | SGTASSSPGSSTPSGATGSP |
| | GSSPSASTGTGPGASPGTSS |
| | TGSPGTPGSGTASSSPGSST |
| | PSGATGSPGTPGSGTASSSP |
| | GASPGTSSTGSPGASPGTSS |
| | TGSPGTPGSGTASSSPGSST |
| | PSGATGSPGASPGTSSTGSP |
| | GTPGSGTASSSPGSSTPSGA |
| | TGSPGSSPSASTGTGPGSSP |
| | SASTGTGPGSSTPSGATGSP |
| | GSSTPSGATGSPGASPGTSS |
| | TGSPGASPGTSSTGSPGASP |
| | GTSSTGSPGTPGSGTASSSP |
| | GASPGTSSTGSPGASPGTSS |
| | TGSPGASPGTSSTGSPGSSP |
| | SASTGTGPGTPGSGTASSSP |
| | GASPGTSSTGSPGASPGTSS |
| | TGSPGASPGTSSTGSPGSST |
| | PSGATGSPGSSTPSGATGSP |
| | GASPGTSSTGSPGTPGSGTA |
| | SSSPGSSTPSGATGSPGSST |
| | PSGATGSPGSSTPSGATGSP |
| | GSSPSASTGTGPGASPGTSS |
| | TGSPGASPGTSSTGSPGTPG |
| | SGTASSSPGASPGTSSTGSP |
| | GASPGTSSTGSPGASPGTSS |
| | TGSPGASPGTSSTGSPGTPG |
| | SGTASSSPGSSTPSGATGSP |
| | GTPGSGTASSSPGSSTPSGA |
| | TGSPGTPGSGTASSSPGSST |
| | PSGATGSPGSSTPSGATGSP |
| | GSSPSASTGTGPGSSPSAST |
| | GTGPGASPGTSSTGSPGTPG |
| | SGTASSSPGSSTPSGATGSP |
| | GSSPSASTGTGPGSSPSAST |
| | GTGPGASPGTSSTGSPGASP |
| | GTSSTGSPGSSTPSGATGSP |
| | GSSPSASTGTGPGASPGTSS |
| | TGSPGSSPSASTGTGPGTPG |
| | SGTASSSPGSSTPSGATGSP |
| | GSSTPSGATGSPGASPGTSS |
| | TGSP |

In some instances, the XTEN is selected from the group consisting of AE42, AG42, AE42 2, AE42 3, AE48, AM48, AE72, AE72 2, AE72 3, AG72, AE108, AG108, AE144, AF144, AE144 2, AE144 3, AG144, AE180, AG180, AE216, AG216, AE252, AG252, AE288, AG288, AE295, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AE872, AE884, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, and AG2004. In some instances, the XTEN is selected from the group consisting of AE42, AE864, AE576, AE288, AE144, AE288, AG864, AG576, AG288, and AG144. In some instances, the XTEN is selected AE144. In some instances, the XTEN is AE288.

Additional examples of XTEN polypeptides that can be used according to the present disclosure are disclosed in U.S. Pat. Nos. 7,855,279 and 7,846,445, US Patent Publication Nos. 2009/0092582, 2010/0239554, 2010/0323956, 2011/0046060, 2011/0046061, 2011/0077199 A1, 2011/0172146, 2012/0178691; 2012/0263701; or 2013/0017997, International Patent Publication Nos. WO 2010091122, WO 2010144502, WO 2010144508, WO 2011028228, WO 2011028229, WO 2011028344; and WO2013130683.

### Human Serum Albumin

In some instances, the chimeric molecules include human serum albumin (HSA) as the fusion moiety instead of an Fc region. It is noted that HSA is not dimerizing moiety, but N- and/or C-terminus fusions are possible. In some embodiments, the C-terminus of an antibody variable domain of this disclosure is linked directly or indirectly (e.g., via a linker or therapeutic agent (e.g., antibody, antibody fragment, peptide, enzyme, antisense oligonucleotide)) to the N-terminus of an HSA polypeptide.

HSA has many desirable pharmaceutical properties. These include: a serum half-life of 19-20 days; solubility of about 300 mg/mL; good stability; ease of expression; no effector function; low immunogenicity; and circulating serum levels of about 45 mg/mL. The crystal structure of HSA without and with ligands, including biologically important molecules such as fatty acids and drugs, or complexed with other proteins is well-known in the art. See, e.g., Universal Protein Resource Knowledgebase P02768; He et al., Nature, 358:209-215 (1992); Sugio et al., Protein Eng., 12:439-446 (1999). According to X-ray crystallographic studies of HSA, this polypeptide forms a heart-shaped protein with approximate dimensions of 80×80×80 Å and a thickness of 30 Å. It has about 67% α-helix but no β-sheet and can be divided into three homologous domains (I-III). Each of these three domains is comprised of two subdomains (A and B). The A and B subdomains have six and four α-helices, respectively, connected by flexible loops. The principal regions of ligand binding to human serum albumin are located in cavities in subdomains IIA and IIIA, which are formed mostly of hydrophobic and positively charged residues and exhibit similar chemistry. All but one of the 35 cysteine residues in the molecule are involved in the formation of 17 stabilizing disulfide bonds. The amino acid sequence as well as the structures of bovine, horse, rabbit, equine and leporine albumins are known. See, e.g., Majorek et al., Mol. Immunol., 52:174-182 (2012); Bujacz, Acta Crystallogr. D Biol. Crystallogr., 68:1278-1289 (2012). Numerous genetic variants of human serum albumin are well-known in the art. See, e.g., The Albumin Website maintained by the University of Aarhus, Denmark and the University of Pavia, Italy at albumin.org/genetic-variants-of-human-serum-albumin and albumin.org/genetic-variants-of-human-serum-albumin-reference-list.

In one embodiment, a human serum albumin used in the chimeric molecule comprises or consists of the amino acid sequence set forth below:

(SEQ ID NO: 94)
DAHKSEVAHRFKDLGEENFKALVLIAFAQYLQQCPF
EDHVKLVNEVTEFAKTCVADESAENCDKSLHTLFG
DKLCTVATLRETYGEMADCCAKQEPERNECFLQHK
DDNPNLPRLVRPEVDVMCTAFHDNEETFLKKYLYE
IARRHPYFYAPELLFFAKRYKAAFTECCQAADKAA
CLLPKLDELRDEGKASSAKQRLKCASLQKFGERAF
KAWAVARLSQRFPKAEFAEVSKLVTDLTKVHTECC
HGDLLECADDRADLAKYICENQDSISSKLKECCEK
PLLEKSHCIAEVENDEMPADLPSLAADFVESKDVC
KNYAEAKDVFLGMFLYEYARRHPDYSVVLLLRLAK
TYETTLEKCCAAADPHECYAKVFDEFKPLVEEPQN
LIKQNCELFEQLGEYKFQNALLVRYTKKVPQVSTP
TLVEVSRNLGKVGSKCCKHPEAKRMPCAEDYLSVV
LNQLCVLHEKTPVSDRVTKCCTESLVNRRPCFSAL
EVDETYVPKEFNAETFTFHADICTLSEKERQIKKQ
TALVELVKHKPKATKEQLKAVMDDFAAFVEKCCKA
DDKETCFAEEGKKLVAASQAALGL

In another embodiment, a human serum albumin used in the chimeric molecule is a HSA variant has an amino acid sequence that is at least 85%, at least 90%, at least 91%, at least 92%, at least 93%, at least 94%, at least 95%, at least 96%, at least 97%, at least 98%, or at least 99% identical to the amino acid sequence set forth in SEQ ID NO:94. Percent identity between amino acid sequences can be determined using the BLAST 2.0 program. Sequence comparison can be performed using an ungapped alignment and using the default parameters (Blossom 62 matrix, gap existence cost of 11, per residue gap cost of 1, and a lambda ratio of 0.85). The mathematical algorithm used in BLAST programs is described in Altschul et al., 1997, Nucleic Acids Research 25:3389-3402.

In certain embodiments, the human serum albumin used in the chimeric molecule is a HSA variant that may have N and/or C-terminal deletions in the sequence of SEQ ID NO:94 (e.g., 1-10, 1-9, 1-8, 1-7, 1-6, 1-5, 1-4, 1-3, 1-2, 1, 2, 3, 4, 5, 6, 7, 8, 9, or 10 consecutive amino acids at the N- and/or C-terminal may be deleted). In some instances, the HSA variant has the same or substantially the same desirable pharmaceutical properties of HSA having the amino acid sequence of SEQ ID NO:94 (e.g., a serum half-life of 19-20 days; solubility of about 300 mg/mL; good stability; ease of expression; no effector function; low immunogenicity; and/or circulating serum levels of about 45 mg/mL). In some instances, the HSA used in the chimeric molecule is a genetic variant of HSA. In some instances, the HSA variant is any one of the 77 variants disclosed in Otagiri et al, 2009, Biol. Pharm. Bull. 32(4), 527-534 (2009). In certain embodiments, the HSA used in the chimeric molecule is a mutated version of HSA that has improved affinity for the neonatal Fc receptor (FcRn) relative to the HSA of SEQ ID NO:94 (see e.g., U.S. Pat. Nos. 9,120,875; 9,045,564; 8,822,417; 8,748,380; Sand et al., Front. Immunol., 5:682 (2014); Andersen et al., J. Biol. Chem., 289(19):13492-502 (2014); Oganesyan et al., J. Biol. Chem., 289(11):7812-24 (2014); Schmidt et al., Structure, 21(11):1966-78 (2013); WO 2014/125082A1; WO 2011/051489, WO2011/124718, WO 2012/059486, WO 2012/150319; WO 2011/103076; and WO 2012/112188, all of which are incorporated by reference herein). In certain instances, the HSA mutant is the E505G/V547A mutant. In certain instances, the HSA mutant is the K573P mutant. Such HSA mutants that HSA that have improved affinity for FcRn can be used to increase the half-life of the chimeric molecule.

### Therapeutic Agent

The chimeric molecule can include a therapeutic agent(s). Examples of therapeutic agents include an antibody, an antigen-binding fragment of an antibody, a single chain antibody (e.g., scFv, sc(Fv)2), a diabody, a nanobody, an antibody fragment, a nucleic acid (e.g., an antisense oligonucleotide), a peptide, or an enzyme, used alone or in combination therapy with standard of care treatments. The chimeric molecules of this disclosure can transport a therapeutic agent across the BBB to the CNS. Thus, the chimeric molecules can be used to treat and/or prevent a neurological disorder. Exemplary neurological disorders include Alzheimer's disease, Parkinson's disease, frontotemporal dementia, ALS, Huntington's disease, multiple sclerosis, spinal muscular atrophy, muscular dystrophy, spinal cord injury, stroke, ophthalmological conditions, acute or chronic optic neuritis, psychiatric disorders, Tourette's disease brain injury, brain tumors, and epilepsy.

Exemplary therapeutic agents for the treatment of Alzheimer's disease include caprylic triglyceride, anti-tau antibody, anti-beta amyloid antibody, anti-DKK1 antibody, APOE antagonist antibody, donepezil, quinidine, a serotonin 6 receptor antagonist, a beta-secretase inhibitor, a RAGE antagonist, a BACE inhibitor, an amyloid beta-protein inhibitor, a phosphodiesterase 9A inhibitor, bisnorcymserine, bryostatin-1, an alpha-7 potentiator, a purinoceptor P2Y6 agonist, a tau protein aggregation/TDP-43 aggregation inhibitor, N3pG-Aβ mAb, an mGlu2 agonist, quinazolinone, a mitochondrial protein stimulant, an amyloid precursor protein secretase inhibitor, a 5HT6 antagonist, R-phenserine, an amyloid beta/tau protein inhibitor, a MAO-B inhibitor, an Lp-PLA2 inhibitor, a 5-HT6 receptor antagonist, a BET protein inhibitor, an anti-protofibrillar AB mAb, nomethiazole, a histamine H3 receptor antagonist, a PPAR-delta/gamma agonist, abeotaxane, and a p38 mitogen-activated protein kinase inhibitor.

Exemplary therapeutic agents for the treatment of ALS include an anti-SOD1 antibody, anti-DR6 antibody, anti-DPR antibody, dexpramipexole, arimoclomal, GM6, ibudilast, a macrophage modulator, a NOGO-A inhibitor, and a troponin complex stimulant.

Exemplary therapeutic agents for the treatment of brain injury include apomorphine, a cytokine inhibitor/neuropeptide receptor modulator, and a progesterone receptor agonist.

Exemplary therapeutic agents for the treatment of brain tumors include an IDH1 inhibitor, doxorubicin, paclitaxel, an anti-EGFRvIII antibody-drug conjugate, bevacizumab, a FGF-R kinase inhibitor, a PI3K inhibitor, cabozantinib, iodine I 131 derlotuximab biotin, a PDGFR inhibitor, carboxyamidotriazole orotate, a non-neurotoxic derivative of penclomidine, golvatinib, dexanabinol, a TGF-beta 1 kinase inhibitor, afatinib, an IDO inhibitor, cabazitaxel, a Src kinase/pre-tubulin inhibitor, a SMO protein inhibitor, an endothelin A/B receptor antagonist, a proteasome inhibitor, a T-type calcium channel antagonist, a thapsigargin analogue, irinotecan, nivolumab, a CSF-1R inhibitor, pelareorep, an EGFR antagonist, an exportin-1 protein inhibitor/nuclear protein inhibitor, a BIRC5 protein inhibitor, evofosfamide, abeotaxane, ENG protein inhibitor, trans-sodium crocetinate, an N7-alkylating agent, a targeted anti-angiogenic agent, and veliparib.

Exemplary therapeutic agents for the treatment of epilepsy include everolimus, eslicarbazepine acetate, alprazolam, brivaracetam, carbamazepine, cannabidiol, a 4-aminobutyrate transaminase inhibitor, perampanel, a GABA-A receptor agonist, synthetic huperzine, pregabalin, clobazam, diazepam, a GABAA synaptic and extra-synaptic receptor modulator, topiramate IV, lacosamide, and a serotonin receptor agonist.

Exemplary therapeutic agents for the treatment of genetic disorders (e.g., Friedrich's ataxia, late infantile neuronal ceroid, spinal and bulbar muscular atrophy, ataxia telangiectasia, pantothenate kinase-associated neurodegeneration, spinal muscular atrophy, familial amyloid polyneuropathy, Rett syndrome, Leigh syndrome, Wilson's disease) include a NF/E2 related factor 2 stimulant, interferon gamma-1b, rhTPP1 enzyme replacement therapy, vatiquinone, deferiprone, Spinraza®, ISIS-TTRRX, a serotonin 1A receptor agonist, cytokine inhibitors/neuropeptide receptor modulator, an siRNA inhibitor targeting TTR, phosphopantothenate replacement, DcpS inhibitor, cysteamine bitartrate, indolepropionic acid, a transthyretin dissociation inhibitor, and bis-choline tetrathiomolybdate.

Exemplary therapeutic agents for the treatment of headache include an anti-CGRP mAb, a CGRP receptor antagonist mAb, sumatriptan, dextromethorphan/quinidine, onabotulinumtoxinA, a serotonin-1F receptor agonist, a nNOS inhibitor/5HT, dihydroergotamine, cyclobenzaprine, and aspirin/sumatriptan combination.

Exemplary therapeutic agents for the treatment of Huntington's disease include laquinimod, a PDE10 inhibitor, pridopidine, cysteamine bitartrate, and aVMAT2 inhibitor.

Exemplary therapeutic agents for the treatment of multiple sclerosis include natalizumab, monomethyl fumarate prodrug, anti-LINGO-1 antibody, a Nck protein modulator, a S1PR-1/5 receptor agonist, fingolimod, an anti-CD52 mAb, idebenone, a PPAR-gamma agonist/modulator, laquinimod, a tyrosine kinase inhibitor, an anti-CD19 mAb, ibudilast, guanabenz, an anti-CD20 mAb, interferon beta-1b, an IL-7 receptor inhibitor, a S1P1 receptor agonist, a myelin protein stimulant, estriol succinate, imilecleucel-T, an anti-VLA 2 mAb, a BAFF-R modulator, a CD100 antigen inhibitor, an anti-DR6 antibody, and an NF-kappa B inhibitor.

Exemplary therapeutic agents for the treatment of muscular dystrophy include a myostatin inhibitor, drisapersen, eteplirsen, halofuginone, idebenone, ISIS-DMPKRx, a (steroid receptor agonist, a GAPDH inhibitor, a genetic transcription inhibitor, tadalafil, ataluren, and a glucocorticoid receptor agonist.

Exemplary therapeutic agents for the treatment of pain include a neublastin, P2X3 purinoreceptor antagonist, a SNARE protein antagonist, oxycodone-naltrexone core (abuse resistant), amitriptyline/ketamine, rintatolimod, a cannabinoid receptor CB2 agonist, a non-eryhropoietic peptide, a PPAR-gamma agonist, a glycogen phosphorylase inhibitor, a NMDA receptor antagonist, zoledronic acid, an early growth response protein 1 inhibitor, a (histamine-3 receptor antagonist, buprenorphine, a cytokine inhibitor, cebranopadol, celecoxib, an arachidonic acid analog, a synthetic capsaicin, a Nav1.7 sodium channel inhibitor, an opioid kappa receptor agonist, duloxetine, a nerve growth factor stimulant, dexmedetomidine, a voltage-gated sodium channel inhibitor, bupivacaine, an angiotensin type 2 receptor antagonist, a nerve growth factor inhibitor, a p38 inhibitor, rapastinel, levorphanol, a CGRP mAb, pregabalin, an mGlu2/3 receptor agonist, a CACNA2D1 protein modulator, a bone resorption factor inhibitor, neublastin, a mu-opioid analgesic, an nNOS inhibitor, O-desmethyltramadol, palmitoylethanolamide, a GABA A agonist, a TRPV-1 receptor agonist, nabiximols, a cyclo-oxygenase 2 inhibitor, a nerve growth factor modulator, cyclobenzaprine, flurbiprofen, a fatty acid amide hydrolase inhibitor, and ibuprofen/phosphatidylcholine.

Exemplary therapeutic agents for the treatment of Parkinson's disease include amantadine, apomorphine, an alpha7 nicotine acetylcholine receptor partial agonist, an anti-alpha-synuclein antibody, alpha-synuclein inhibitor, levodopa, a D1 potentiator, dipraglurant, a serotonin 1A/1B partial agonist, fipamezole, GM6, a retinoid X receptor agonist, istradefylline, rotigotine, pramipexole/rasagiline, R-phenserine, a serotonin 2A/6 receptor antagonist, an adenosine A2A receptor antagonist, safinamide, and a dopamine receptor agonist.

Exemplary therapeutic agents for the treatment of spasticity include baclofen, onabotulinumtoxinA, abobotulinumtoxinA, arbaclofen, nabiximols, and incobotulinumtoxinA.

Exemplary therapeutic agents for the treatment of Spinal Cord Injury include an anti-Lingo-1 antibody, anti-NgR1 antibody, neublastin, a nervous system modulator, a Rho GTP-binding protein-inhibitor, and fibroblast growth factor receptor.

Exemplary therapeutic agents for the treatment of stroke include natalizumab, recombinant mutant form of human wild-type activated protein C, ticagrelor, dalfampridine, aspirin, nimodipine microparticles, GM6, a PARP inhibitor, a PDZ domain inhibitor, a beta amyloid inhibitor, dabigatran, and sodium nitrite.

Exemplary therapeutic agents for the treatment of Tourette's Syndrome include a histamine-3 receptor antagonist, a 4-aminobutyrate transaminase inhibitor, abobotulinumtoxinA, ecopipam, a VMAT2 inhibitor, acamprosate, and vigabatrin.

Other exemplary therapeutic agents for the treatment of other neurological disorders include a myostatin inhibitor, NF/E2 related factor 2 stimulant, anti-tau antibody, a myeloperoxidase inhibitor, a mitochondrial permeability transition pore inhibitor, belimumab, type II-B activin receptor modulator mAb, a C1 esterase inhibitor, ferric carboxymaltose, amifampridine, fingolimod, a monoamine oxidase B inhibitor, a neurotransmitter modulator, a dopamine receptor agonist, an anti-CD19 mAb, a VMAT2 inhibitor, a CD20 mAb, thymosin beta-4, an anti-IL-6 receptor mAb, eculizumab, an AMPA receptor modulator, a steroid hydroxylase inhibitor, pyridoxal phosphate, abeotaxane, aceneuramic acid, and sodium oxybate.

In one instance, the therapeutic agent is an antibody. Non-limiting examples of the CDRs of exemplary therapeutic antibodies are provided below.

| Antibody | VH-CDR1 | VH-CDR2 | VH-CDR3 |
|---|---|---|---|
| LINGO-1 | AYEMK | VTGPS | EGDND |
| | (SEQ ID | GGFTF | AFDI |
| | NO: 122) | YADSV | (SEQ ID |
| | | KG | NO: 124) |
| | | (SEQ ID | |
| | | NO: 123) | |
| TWEAK | GFTFS | EISSG | VLYYD |
| | RYAMS | GSYPY | YDGDR |
| | (SEQ ID | YPDTV | IEVMD |
| | NO: 128) | TG | Y |
| | | (SEQ ID | (SEQ ID |
| | | NO: 129) | NO: 130) |
| Beta- | SYGMH | VIWFD | DRGIG |
| amyloid | (SEQ ID | GTKKY | ARRGP |
| | NO: 134) | YTDSV | YYMDV |
| | | KG | (SEQ ID |
| | | (SEQ ID | NO: 136) |
| | | NO: 135) | |
| tau | GFTFS | VIWFD | DLGAS |
| | SYDMH | GSNEF | VTTSN |
| | (SEQ ID | YADSV | AENFH |
| | NO: 140) | KG | H |
| | | (SEQ ID | (SEQ ID |
| | | NO: 141) | NO: 142) |
| tau | GFTFP | SGISG | GSGGI |
| | NYVMT | SGGST | (SEQ ID |
| | (SEQ ID | DYADS | NO: 151) |
| | NO:149) | VKG | |
| | | (SEQ ID | |
| | | NO: 150) | |
| tau | KYGMS | TISSS | SWDGA |
| | (SEQ ID | GSRTY | MDY |
| | NO: 155) | YPDSV | (SEQ ID |
| | | KG | NO: 157) |
| | | (SEQ ID | |
| | | NO: 156) | |
| Alpha- | KAWMS | RIKST | AH |
| synuclein | (SEQ | ADGGT | (SEQ ID |
| | ID | TSYAA | NO: 164) |
| | NO: | PVEG | |
| | 161) | (SEQ ID | |
| | or | NO: 163) | |
| | GFDFE | | |
| | KAWMS | | |
| | (SEQ ID | | |
| | NO: 162) | | |
| TDP-43 | GFTFS | NIKQD | PPGW |
| | TYYMS | GSEKY | (SEQ ID |
| | (SEQ ID | YVDSV | NO: 170) |
| | NO: 168) | KG | |
| | | (SEQ ID | |
| | | NO: 169) | |
| C9orf72 | GFTFS | VISYD | GGRRG |
| DPR | NHAMH | GENTY | HFTSY |
| mAbl | (SEQ ID | YADSI | YLDY |
| | NO: 201) | EG | (SEQ ID |
| | | (SEQ ID | NO: 203) |
| | | NO: 202) | |
| C9orf72 | GGSVS | RTYTN | WGAVT |
| DPR | D | GKTTY | GDYYY |
| mAb2 | (SEQ ID | TYNPS | GMDV |
| | NO: 174) | LES | (SEQ ID |
| | | (SEQ ID | NO: 176) |
| | | NO: 175) | |
| Antibody | VL-CDR1 | VL-CDR2 | VL-CDR3 |
| LINGO-1 | RASQS | DASNR | QQRSN |
| | VSSYL | AT | WPMYT |
| | A | (SEQ ID | (SEQ ID |
| | (SEQ ID | NO: 126) | NO: 127) |
| | NO: 125) | | |
| TWEAK | RSSQS | KVSNR | SQSTH |
| | LVSSK | FS | FPRT |
| | GNTYL | (SEQ ID | (SEQ ID |
| | H | NO: 132) | NO: 133) |
| | (SEQ ID | | |
| | NO: 131) | | |
| Beta- | RASQS | AASSL | QQSYS |
| amyloid | ISSYL | QS | TPLT |
| | N | (SEQ ID | (SEQ ID |
| | (SEQ ID | NO: 138) | NO: 139) |
| | NO: 137) | | |
| tau | SGDAL | EDSKR | YSTDS |
| | PKRYV | PS | NGHHW |
| | Y | (SEQ ID | V |
| | (SEQ ID | NO: 144) | (SEQ ID |
| | NO: 143) | | NO: 145) |
| tau | TGTSS | DVTKR | HSYVG |
| | DVGGY | PS | SYTLV |
| | NYVS | (SEQ ID | (SEQ ID |
| | (SEQ ID | NO: 153) | NO: 154) |
| | NO: 152) | | |
| tau | KSSQS | KVSNR | FQGSL |
| | IVHSN | FS | VPWA |
| | GNTYL | (SEQ ID | (SEQ ID |
| | E | NO: 159) | NO: 160) |
| | (SEQ ID | | |
| | NO: 158) | | |
| Alpha- | SGEAL | KDSER | QSPDS |
| synuclein | PMQFA | PS | TNTYE |
| | H | (SEQ ID | V |
| | (SEQ ID | NO: 166) | (SEQ ID |
| | NO: 165) | | NO: 167) |
| TDP-43 | KSSQS | EVSNR | MQSIQ |
| | LLHSD | FS | LPVT |
| | GKTYL | (SEQ ID | (SEQ ID |
| | Y | NO: 172) | NO: 173) |
| | (SEQ ID | | |
| | NO: 171) | | |
| C9orf72 | RASQN | AASSL | QQSYS |
| DPR | IDKYL | HS | SFRT |
| mAbl | N | (SEQ ID | (SEQ ID |
| | (SEQ ID | NO: 178) | NO: 179) |
| | NO: 177) | | |
| C9orf72 | RSPRS | LASNR | MQGLQ |
| DPR | LLHTN | AS | PSWT |
| mAb2 | GYT LD | (SEQ ID | (SEQ ID |
| | (SEQ ID | NO:181) | NO: 182) |
| | NO: 180) | | |

Non-limiting examples of the VH and VL of exemplary therapeutic antibodies are provided below.

Anti-LINGO-1:
VH:
(SEQ ID NO: 183)
EVQLLESGGG LVQPGGSLRL SCAASGFTFS AYEMKWVRQA
PGKGLEWVSV IGPSGGFTFY ADSVKGRFTI SRDNSKNTLY
LQMNSLRAED TAVYYCATEG DNDAFDIWGQ GTTVTVSS
VL:
(SEQ ID NO: 184)
DIQMTQSPAT LSLSPGERAT LSCRASQSVS SYLAWYQQKP
GQAPRLLIYD ASNRATGIPA RFSGSGSGTD FTLTISSLEP
EDFAVYYCQQ RSNWPMYTFG QGTKLEIK
Anti-TWEAK:
VH:
(SEQ ID NO: 185)
EVQLVESGGG LVQPGGSLRL SCAASGFTFS RYAMSWVRQA
PGKGLEWVAE ISSGGSYPYY PDTVTGRFTI SRDNAKNSLY
LQMNSLRAED TAVYYCARVL YYDYDGDRIE VMDYWGQGTL
VTVSS
VL:
(SEQ ID NO: 186)
DVVMTQSPLS LPVTPGEPAS ISCRSSQSLV SSKGNTYLHW
YLQKPGQSPQ LLIYKVSNRF SGVPDRFSGS GSGTDFTLKI
SRVEAEDVGV YYCSQSTHFP RTFGGGTKVE IK
Anti-Beta-Amyloid:
VH:
(SEQ ID NO:187)
QVQLVESGGGVVQPGRSLRLSCAASGFAFSSYGMHWVRQA
PGKGLEWVAVIWFDGTKKYYTDSVKGRFTISRDNSKNTLY
LQMNTLRAEDTAVYYCARDRGIGARRGPYYMDVWGKGTTV
TVSS
VL:
(SEQ ID NO: 188)
DIQMTQSPSSLSASVGDRVTITCRASQSISSYLNWYQQKP
GKAPKLLIYAASSLQSGVPSRFSGSGSGTDFTLTISSLQP
EDFATYYCQQSYSTPLTFGGGTKVEIKR
Anti-Tau:
VH:
(SEQ ID NO: 189)
QVQLVESGGGVVQPGRSLRVSCAASGFTFSSYDMHWVRQA
PGKGLEWVAVIWFDGSNEFYADSVKGRFTISRDNSKNTLF
LQMNSLRAEDTAVYYCARDLGASVTTSNAENFH
HWGQGTLVTVSS
VL:
(SEQ ID NO: 190)
SYELTQPPSVSVSPGQTARITCSGDALPKRYVYWYQQKS
GQAPVLVIYEDSKRPSGIPETFSGSSSGTMATLTISGAQ
VEDEADYYCYSTDSNGHHWVFGGGTKLTVL
Anti-Tau:
VH:
(SEQ ID NO: 191)
EVQLVESGGGLVQPGGSLRLSCAASGFTFPNYVMTWVRQ
APGKGLEWVSGISGSGGSTDYADSVKGRFTISRDNSKNT
LYLQMNSLRVEDTALYYCAKGSGGIRGQGTMVTVSS
VL:
(SEQ ID NO: 192)
QSALTQPRSVSGSPGQSVTISCTGTSSDVGGYNYVSWHQ
QHPGKAPKLLIYDVTKRPSGVPDRFSGSKSGNTAALTIS
GLQAEDEADYYCHSYVGSYTLVFGGGTKLTVL
Anti-Tau:
VH:
(SEQ ID NO: 193)
EVHLVESGGA LVKPGGSLRL SCAASGFSFS KYGMSWVRQA
PGKGLEWVAT ISSSGSRTYY PDSVKGRFTI SRDNAKNTLY
LQMNSLRAED TAMYYCSISW DGAMDYWGQG TTVTVSS
VL:
(SEQ ID NO: 194)
DVVMTQSPLS LPVTLGQPAS ISCKSSQSIV HSNGNTYLEW
YLQKPGQSPQ LLVYKVSNRFSGVPDRFSGS GSGTDFTLKI
SRVEAEDVGT YYCFQGSLVP WAFGGGTKVE IK
Anti-Alpha-Synuclein:
VH:
(SEQ ID NO: 195)
EVQLVESGGG LVEPGGSLRL SCAVSGFDFE KAWMSWVRQA
PGQGLQWVAR IKSTADGGTT SYAAPVEGRF IISRDDSRNM
LYLQMNSLKT EDTAVYYCTS AHWGQGTLVT VSS
VL:
(SEQ ID NO: 196)
SYELTQPPSV SVSPGQTARI TCSGEALPMQ FAHWYQQRPG
KAPVIVVYKD SERPSGVPER FSGSSSGTTA TLTITGVQAE
DEADYYCQSP DSTNTYEVFG GGTKLTVL
Anti-C9orf72 DPR:
VH1:
(SEQ ID NO: 197)
QVQLVESGGGVVQPGRSLRLSCAASGFTFSNHAMHWVR
QAPGKGLEWVAVISYDGENTYYADSIEGRFTISRDNFK
NTLFLQMYSLTADDTAMYFCARGGRRGHFTSYYLDY
WGQGTLVTVSS
VL1:
(SEQ ID NO: 198)
DIQMTQSPSSLSASVGDRVTITCRASQNIDKYLNWYQQ
IPGKAPKLLIYAASSLHSGVPSRFSGSGSGTDFSLTIS
SLQPEDFAIYYCQQSYSSFRTFGQGTKLEIK
VH2:
(SEQ ID NO: 199)
QVQLQESGPGLVKPSETLSLTYTVLGGSVSDYYWSCIR
QPAGKGLEWIGRTYTNGKTTYTYNPSLESRLSLSIDTS
MNQFSLKLTSVTAADTAVYYCARWGAVTGDYYYGMDVW
GPGTLVTVSS
VL2:
(SEQ ID NO: 200)
EIVLTQSPLSLSVTPGEPASISCRSPRSLLHTNGYTYL
DWYLQRPGQSPQLLIFLASNRASGVPDRFSGSGSGTNF
TLRISGVEADDVGVYYCMQGLQPSWTFGQGTKVEIK

In some embodiments, the chimeric molecule is a fusion polypeptide comprising an antibody variable domain described above and a whole antibody (the therapeutic agent) (see, FIG. 48). The C-terminus of an antibody variable domain described herein is linked directly or via a linker (e.g., G, GG, G4S (SEQ ID NO:5), 3X G4S (SEQ ID NO:60) to the N-terminus of both the VH or both the VL domains of the antibody. In certain embodiments, the whole antibody is an anti-beta amyloid antibody, an anti-tau antibody, an anti-alpha synuclein antibody, an anti-TDP-43 antibody, an anti-LINGO-1 antibody, an anti-LINGO-2 antibody, an anti-LINGO-3 antibody, an anti-LINGO-4 antibody, an anti-TREM2 antibody, an anti-C9orf72 dipeptide repeat poly-GA antibody (i.e., antibody capable of binding a dipeptide repeat (DPR) of poly-glycine-alanine (GA) having at least 6 repeats (GA)6 as translated from the chromosome 9 open reading frame 72 (C9orf72) gene, an anti-TWEAK antibody, or an anti-TWEAK-R antibody.

In some embodiments, the chimeric molecule comprises an antibody variable domain described above, a hinge region, an Fc region, and an Fab (therapeutic agent) (see, FIG. 48). The C-terminus of the antibody variable domain is linked directly or indirectly to the N-terminus of each hinge region which in turn is linked directly or indirectly to the Fc region. The C-terminus of each Fc domain of the Fc region is linked via a linker (e.g., G, GG, G₄S (SEQ ID NO:5), 3X G₄S (SEQ ID NO:60) to the N-terminus of the VH or VL domain of the Fab (therapeutic agent). In certain embodiments, the Fab is an anti-beta amyloid Fab, an anti-tau Fab, an anti-alpha synuclein Fab, an anti-TDP-43 Fab, an anti-LINGO-1 Fab, an anti-LINGO-2 Fab, an anti-LINGO-3 Fab, an anti-LINGO-4 Fab, an anti-TREM2 Fab, an anti-C9orf72 dipeptide repeat poly-GA Fab, an anti-TWEAK Fab, or an anti-TWEAK-R Fab.

In certain embodiments, the chimeric molecule comprises an antibody variable domain described herein, a therapeutic agent, a hinge region, and an Fc region. The C-terminus of the antibody variable domain is linked directly or via a linker (e.g., G, GG, G₄S (SEQ ID NO:5), 3X G4S (SEQ ID NO:60) to the N-terminus of the therapeutic agent (e.g., an antibody fragment (e.g., Fab)), an enzyme, a peptide). The C-terminus of the therapeutic agent is linked directly or via a linker to the N-terminus of a hinge region. The C-terminus of the hinge region is linked to the N-terminus of each Fc domain of the Fc region. In some cases, the

In some embodiments, the chimeric molecule comprises an antibody variable domain described herein, a therapeutic agent, a hinge region, and an Fc region. The C-terminus of the antibody variable domain is linked directly or via a linker to the N-terminus of a hinge region. The C-terminus of the hinge region is linked to the N-terminus of each Fc domain of the Fc region. The N-terminus of the therapeutic agent (e.g., an antibody fragment (e.g., Fab)), an enzyme, a peptide) is linked directly or via a linker (e.g., G, GG, G₄S (SEQ ID NO:5), 3X G₄S (SEQ ID NO:60) to the C-terminus of one or both Fc domains of the Fc region.

In certain embodiments, the chimeric molecule comprises an antibody variable domain described herein, an ASO (therapeutic agent), a hinge region, and an Fc region. The C-terminus of the antibody variable domain is linked directly or via a linker to the N-terminus of a hinge region. The C-terminus of the hinge region is linked to the N-terminus of each Fc domain of the Fc region. The ASO is linked to the hinge region. In certain cases, the ASO is a splice switching oligonucleotide. In other instances, the ASO is a gapmer.

In some cases, the chimeric molecule comprises an antibody variable domain described herein, a hinge region, an Fc region, and a lipid nanoparticle, a liposome, or a polymeric nanocarrier which encapsulates a therapeutic agent (e,g., an ASO (e.g., a splice switching oligonucleotide or a gapmer), a small molecule drug, a nucleic acid, a peptide). The C-terminus of the antibody variable domain is linked directly or via a linker to the N-terminus of a hinge region. The C-terminus of the hinge region is linked to the N-terminus of each Fc domain of the Fc region. The liposome may be linked to the C-terminus of one or both Fc domains of the Fc region. Methods of nanoscale delivery are well known in the art. See. e.g., Juliano et al., Nucl. Acids Res., 44(14) (2016) and the references cited therein, which are all incorporated by reference in their entireties herein.

It should be understood that the chimeric molecules described above can include a HSA moiety instead of an Fc region.

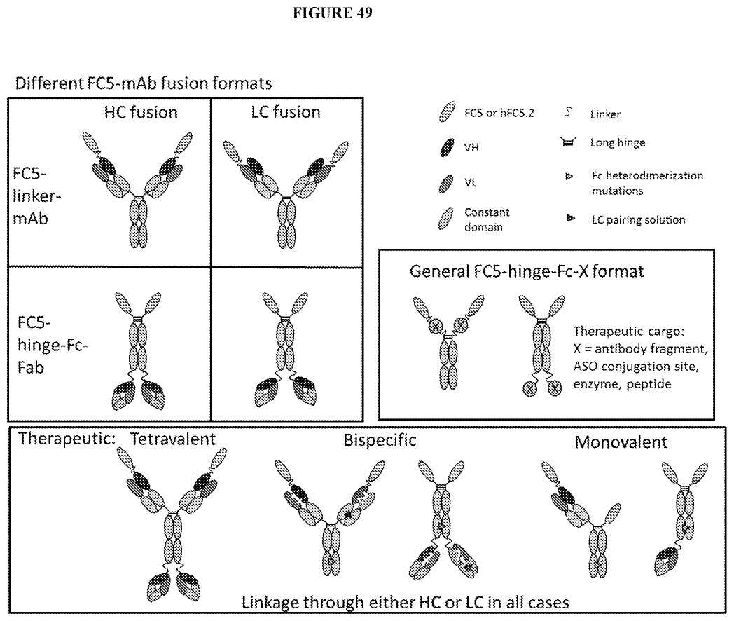

In addition, the disclosure encompasses monovalent, bispecific, and tetravalent designs as shown in FIG. 49.

### Nucleic Acids, Vectors, Host Cells, and Methods of Making the Polypeptides

This disclosure also encompasses nucleic acid encoding the antibody variable domains disclosed above. In addition, encompassed are nucleic acid or nucleic acids encoding the fusion polypeptides described herein. The nucleic acid or nucleic acids can be inserted into a vector or vectors (e.g., expression vectors).

The nucleic acids encoding the antibody variable domains and fusion polypeptides comprising same that are described above can be expressed in any desired host cell (e.g., bacterial cells, yeast cells, mammalian cells). In certain embodiments, the polypeptide is secreted from the host cell. In one embodiment, the host cell is a mammalian cell. In a specific embodiment, the host cell is a CHO cell or cell line. In some instances, an antibody variable domain polypeptide coding sequence (e.g., humanized FC5 or a disulfide stabilized and humanized FC5) is fused to an Fc (e.g., IgG1, agly IgG1, IgG2, IgG3, IgG4) coding sequence via an intervening amino acid sequence. In some instances, the intervening amino acid sequence comprises a hinge sequence (e.g., a hinge from IgG1, IgG4, etc . . . ). In some instances, the intervening amino acid sequence comprises a Fab and a hinge sequence

If the polypeptide is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., Nature, 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for secretion. For production into the periplasm of E. coli, the pelB signal sequence (Lei et al., J. Bacteriol., 169:4379 (1987)) may be used as the signal sequence for secretion. For bacterial expression, calcium chloride methods or electroporation methods may be used to introduce the expression vector into the bacterial cell.

If the polypeptide is to be expressed in yeast cells (e.g., Saccharomyces cerevisiae, Saccharomyces italicus, Saccharomyces rouxii, Pichia pastoris, Pichia angusta, Pichia anomala, Pichia capsulate, Kluyveromyces lactis, or yeasts of the genera Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Borryoascus, Sporidiobolus, or Endomycopsis), the expression vector includes a promoter that drives expression of the polypeptide in the yeast cells and/or signal sequences effective for directing secretion in yeast. Suitable promoters for Saccharomyces include those associated with the PGK1 gene, GAL1 or GAL10 genes, CYC1, PHOS, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRB1 promoter, the GUT2 promoter, the GPD1 promoter, and hybrid promoters involving hybrids of parts of 5′ regulatory regions with parts of 5′ regulatory regions of other promoters or with upstream activation sites (e.g. the promoter described in EP-A-258 067). Suitable promoters for Pichia include AOX1, AOX2, MOX1 and FMD1. In some instances, the signal sequence is a yeast-derived signal sequence (e.g., one which is homologous to the yeast host).

If the polypeptide is to be expressed in cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the polypeptide, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

The polypeptide can also be expressed in human cells such as HEK-293 cells.

Variant FC5 polypeptides (and fusions thereof), can be constructed using any of several methods known in the art. One such method is site-directed mutagenesis, in which a specific nucleotide (or, if desired a small number of specific nucleotides) is changed in order to change a single amino acid (or, if desired, a small number of predetermined amino acid residues) in the encoded variant FC5 polypeptide. Many site-directed mutagenesis kits are commercially available. One such kit is the “Transformer Site Directed Mutagenesis Kit” sold by Clontech Laboratories (Palo Alto, Calif.).

FC5 polypeptides and fusions thereof can be produced and isolated using methods well-known in the art. In some embodiments, such polypeptides are produced by recombinant DNA techniques. For example, a nucleic acid molecule(s) encoding a FC5 polypeptide or fusion thereof can be inserted into a vector(s), e.g., an expression vector, and the nucleic acid can be introduced into a cell. Suitable cells include, e.g., mammalian cells (such as human cells or CHO cells), fungal cells, yeast cells, insect cells, and bacterial cells. When expressed in a recombinant cell, the cell is preferably cultured under conditions allowing for expression of the polypeptide. The polypeptide can be recovered from a cell suspension if desired. As used herein, “recovered” means that the mutated polypeptide is removed from those components of a cell or culture medium in which it is present prior to the recovery process. The recovery process may include one or more refolding or purification steps. Methods for isolation and purification commonly used for protein purification may be used for the isolation and purification of the polypeptides described herein, and are not limited to any particular method. Polypeptides may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, metal-chelating chromatography, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column, Capture Select HSA, and Heparin Sepharose. Examples of columns using protein A column include Hyper D, POROS, and Sepharose FF (GE Healthcare Biosciences). The present disclosure also includes polypeptides that are highly purified using these purification methods.

### Methods of Treatment

The antibody variable domains described herein can be used to transport a cargo (e.g., a therapeutic agent) that is useful to treat and/or prevent a disease or disorder. The therapeutic agent may be, e.g., an antibody, an antibody fragment (e.g., an Fab), a nanobody, a diabody, a single chain antibody, a small molecule drug, a peptide, an enzyme, or a nucleic acid (e.g., an ASO). In certain instances, the therapeutic agent is linked directly or indirectly to the antibody variable domain. In other instances, the therapeutic agent is encapsulated in a liposome, lipid nanoparticle, or polymeric nanocarrier that is then linked directly or indirectly to the antibody variable domain. By linking the therapeutic agent to be targeted to the central nervous system to an antibody variable domain described herein, such a fusion can transport the therapeutic agent across the BBB.

In certain embodiments, the disease or disorder is a neurological disorder. In some instances, the disease or disorder is a tauopathy. In some instances, the disease or disorder is a synucleinopathy.

In some instances, the disease or disorder is selected from the group consisting of Alzheimer's disease, Parkinson's disease, multiple sclerosis, frontotemporal dementia (FTD), amyotrophic lateral sclerosis (ALS), progressive supranuclear palsy (PSP), spinal cord injury (SCI), spinal muscular atrophy (SMA), neurological cancers, acute or chronic optic neuritis, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, subacute sclerosing panencephalitis, Tangle only dementia, or multi-infarct dementia.

In other instances, the disease or disorder is selected from the group consisting of stroke, chronic traumatic encephalopathy, traumatic brain injury (TBI), concussion, seizure, epilepsy, or acute lead encephalopathy.

Exemplary therapeutic antibodies that can be employed are disclosed in U.S. Pat. Nos. 8,906,367, 9,605,059; 9,598,484; 9,587,014; 9,777,058; 9,567,395; 8,128,926; 8,058,406; 8,048,422; and U.S. Appl. No. 2017/0247471-A1, all of which are incorporated by reference herein in their entireties.

Exemplary therapeutic ASOs that can be employed are disclosed in U.S. Pat. Nos. 8,361,977 and 8,980,853, both of which are incorporated by reference herein in their entireties.

Therapeutically effective amounts of the composition may be administered to a human subject in need thereof in a dosage regimen ascertainable by one of skill in the art. The frequency of dosing for the composition is within the skill and clinical judgement of physicians. Typically, the administration regime is established by clinical trials which may establish optimal administration parameters. However, the practitioner may vary such administration regimes according to the subject's age, health, weight, sex and medical status. The frequency of dosing may also vary between acute and chronic treatments for the disease or disorder. In addition, the frequency of dosing may be varied depending on whether the treatment is prophylactic or therapeutic.

### Assay

The disclosure also features an assay for assessing the lability of an antibody variable domain. In certain instances, the antibody variable domain is a single domain antibody. In one instance, the single domain antibody is FC5 or a variant thereof (e.g., humanized, disulfide-stabilized, or humanized and disulfide-stabilized).

The method involves providing adding an antibody variable domain to a serum sample to create a mixture; incubating the mixture; purifying the antibody variable domain; and performing peptide mapping. In some instances, the serum sample is rat serum. In other instances, the serum sample is human serum.

In certain embodiments, the antibody variable domain is in the serum at a concentration of 0.01 to 0.5 mg/mL. In some embodiments, the antibody variable domain is in the serum at a concentration of about 0.1 mg/mL. In some embodiments, the antibody variable domain is in the serum at a concentration of 0.05 mg/mL. In some embodiments, the antibody variable domain is in the serum at a concentration of 0.1 mg/mL. In some embodiments, the antibody variable domain is in the serum at a concentration of 0.2 mg/mL. In some embodiments, the antibody variable domain is in the serum at a concentration of 0.3 mg/mL. In some embodiments, the antibody variable domain is in the serum at a concentration of 0.4 mg/mL. In some embodiments, the antibody variable domain is in the serum at a concentration of 0.5 mg/mL.

In certain cases, the mixture is incubated at 25 to 50° C. for 10 to 100 hours. In certain cases, the mixture is incubated at about 37° C. for about 70 hours. In one embodiment, the mixture is incubated at 37° C. for 70 hours.

Example 3 of the disclosure provides a non-limiting way of performing this assay.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: Production of FC5-Anti-LINGO-1 Antibody (Li81) Fusion Proteins

To assess if an FC5 Li81 bispecific antibody could be generated that contained FC5 transport activity and Li81 function we designed, expressed, purified, and characterized the 9 different versions listed in Table 1. Schematics of the designs are shown in the column labeled structures and the ratios of FC5 Vhhs and Li81 valency in each construct are listed in the column labeled FC5:Li81. The constructs contained 1, 2, or 4 FC5 Vhh moieties, and 1 or 2 Li81 Fabs. In the first 4 constructs listed, FC5 was attached to the N-terminus of Li81 heavy chain, light chain, or both, using 1 or 3 G₄S (SEQ ID NO:5) linkers to connect the domains as noted. In the 5th and 6th constructs FC5 was directly attached to the hinge-Fc of the Li81 mAb and the Li81 Fab was fused to the C-terminus of the Fc through its heavy chain. Two different version of the hinge were used to connect the FC5Vhh and the Fc domains noted as long and short. The four other versions were heterodimers as noted where each arm carried a different functionality.

TABLE 1
List of Constructs

| | Name used in | | MW | | |
|---|---|---|---|---|---|
| Li81 Constructs | next slide | Structures | (kDa) | FC5:Li81 | ID# |
| Li81wt(H)FC5(L) | FC5-3x-Li81 LC | 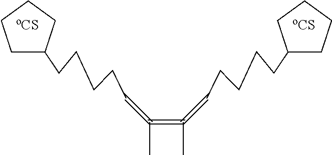 | 175 | 2:2 | 3014 |
| Li81 FC5 (H)wt(L) | FC5-3x-Li81 HC | 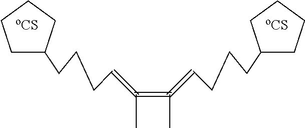 | 175 | 2:2 | 3015 |
| Li81 FC5 (H)FC5(L) | FC5-3x-Li81 HC/LC | 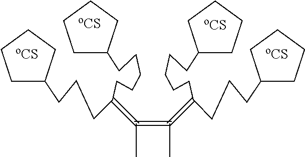 | 205 | 4:2 | 3016 |
| FC5-(G4S)1-L181 hIgG1 agly | FC5-1x-Li81 HC | 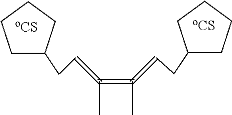 | 175 | 2:2 | 3438 |
| FC5-Fc-(G4S)1-Li81 hIgG1 agly long hinge | FC5(Ih)Fc-1x-Li81 | 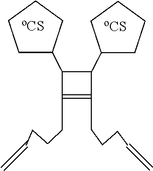 [SMILES: C=CCCCC1=C(CCCC=C)C(C2CCCC2)C1C1CCCC1] | 175 | 2:2 | 3440 |
| FC5-Fc-(G4S)1-Li81 hIgG1 agly short hinge | FC5(sh)Fc-1x-Li81 | 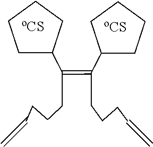 [SMILES: C=CCCC/C(=C(\CCCC=C)C1CCCC1)C1CCCC1] | 175 | 2:2 | 3441 |
| dual-monovalent FC5 Fc-(G4S)1-Li81 hIgG1 agly long hinge | Heterodimer FC5(lh)Fc-1x- Li81 + (lh)Fc | 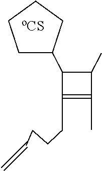 [SMILES: C=CCCCC1=C(C)C(C)C1C1CCCC1] | 115 | 1:1 | 3442 |
| dual-monovalent FC5 Fc-(G4S)1-Li81 hIgG1 agly short hinge | Heterodimer FC5(sh)Fc-1x- Li81 + (sh)Fc | 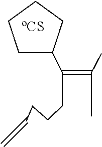 | 115 | 1:1 | 3443 |
| monoFab FC5 Fc- (G4S)1-Li81 hIgG1 agly short hinge | Heterodimer FC5(sh)Fc-1x- Li81 + FC5(sh)Fc | 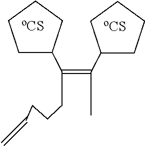 [SMILES: C=CCCC/C(=C(\C)C1CCCC1)C1CCCC1] | 130 | 2:1 | 3444 |

The FC5 Li81 constructs were expressed in CHO cells. For preparation of the conditioned medium, transfected CHO cells were expanded in serum-free media, grown to high density, fed with supplements, and shifted to a reduced temperature. Cultures were held at this reduced temperature for 11-14 days and then harvested by centrifugation and clarified by 0.45-micron filtration.

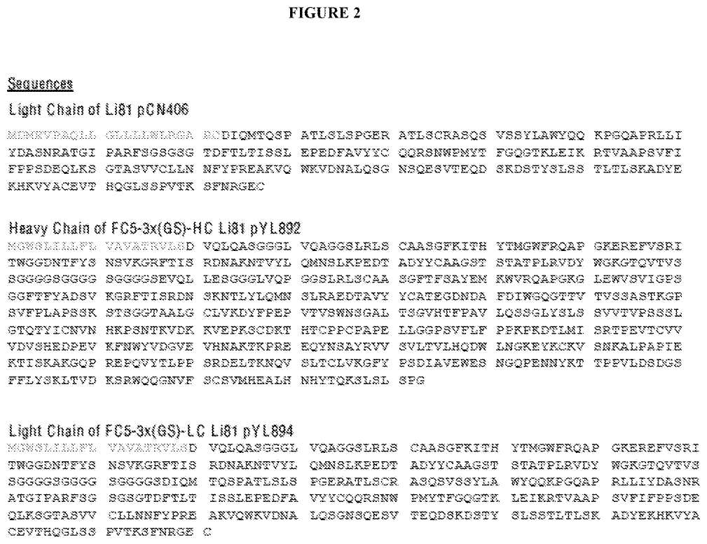

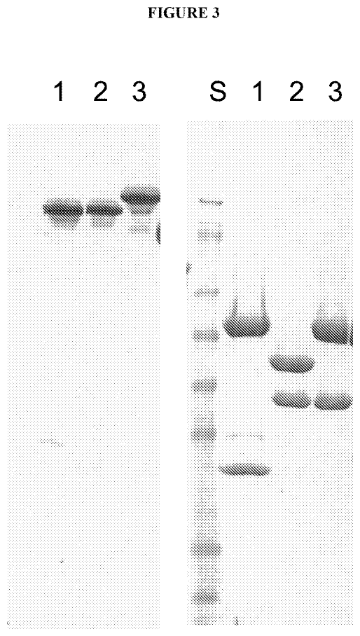

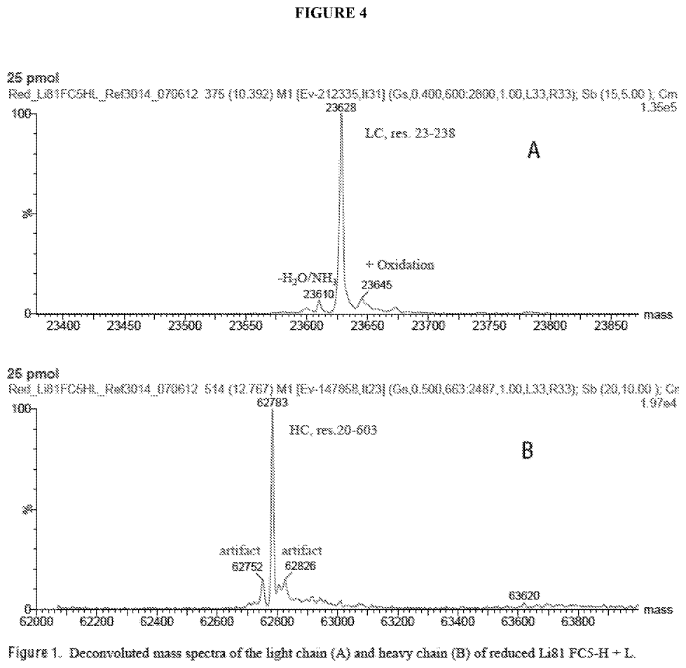

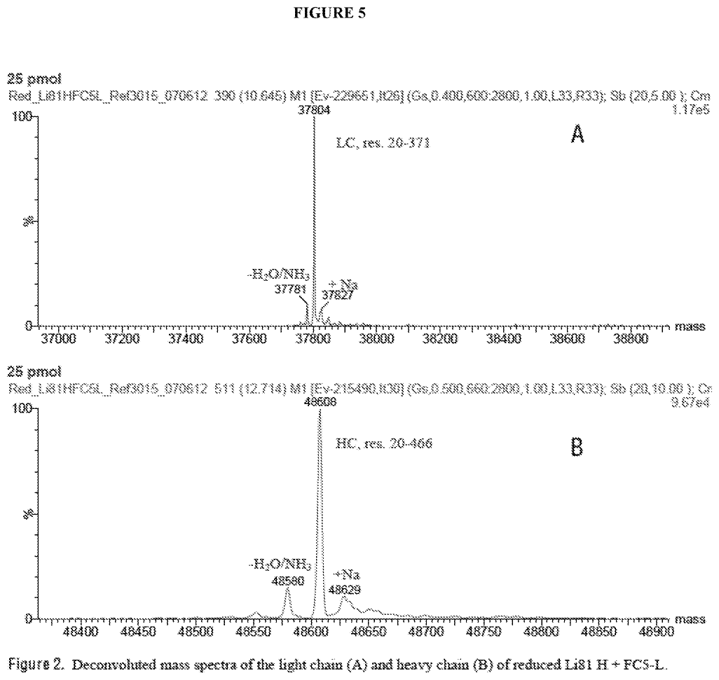

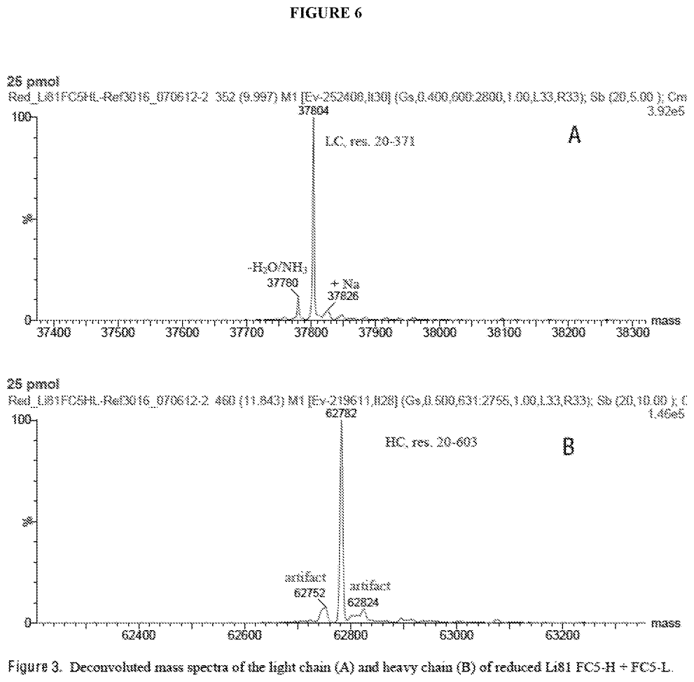

For purification, clarified and filtered culture media were loaded onto protein A columns, washed with 20 mM NaH₂PO₄ pH 7.4, 100 mM NaCl, and then with 25 mM NaH₂PO₄ pH 5.5, 100 mM NaCl. FC5-Li81 was eluted from the columns with 25 mM Na₂HPO₄ pH 2.8, 100 mM NaCl pH 2.8 and neutralized with 25 mM Na₂HPO₄ pH 8.6 diluted from a 0.5 M stock solution. The protein content of the eluted samples was estimated from the absorbance at 280 nm using an extinction coefficient of 1.4 for a 1 mg/mL solution. FIG. 3 shows an SDS-PAGE analysis of the first 3 constructs in which FC5 was fused to the N-terminus of Li81 mAb with 3×G₄S (SEQ ID NO:6) linkers connecting FC5 to the N-terminus of the HC (ID #3014), LC (ID #3015) or both (ID #3016). This analysis confirms the expression of the HC and LC and assembly into the characteristic HC2LC2 tetrameric complex under non-reducing conditions. The slightly larger size of the protein in lane 3 is consistent with the presence of 4 copies of FC5 versus 2 copies in the other samples. FIG. 2 shows amino acid sequences of heavy and light chains used in the three constructs. Intact mass analysis of the reduced heavy chain 3014 (predicted mass 62784. 5 Da, observed mass 62783 Da), reduced light chain 3014 (predicted mass 23628.4 Da, observed mass 23628 Da), reduced heavy chain 3015 (predicted mass 48608.9 Da, observed mass 48608 Da), reduced light chain 3015 (predicted mass 37804.0 Da observed mass 37804 Da) reduced heavy chain 3016 (predicted mass 62784. 5 Da, observed mass 62782 Da), and reduced light chain 3016 (predicted mass 37804.0 Da, observed mass 37804 Da) showed that the major components in all samples were the predicted light chain and heavy chain. The corresponding deconvoluted spectra are shown in FIGS. 4, 5, and 6. No unexpected modifications on the major component were observed.

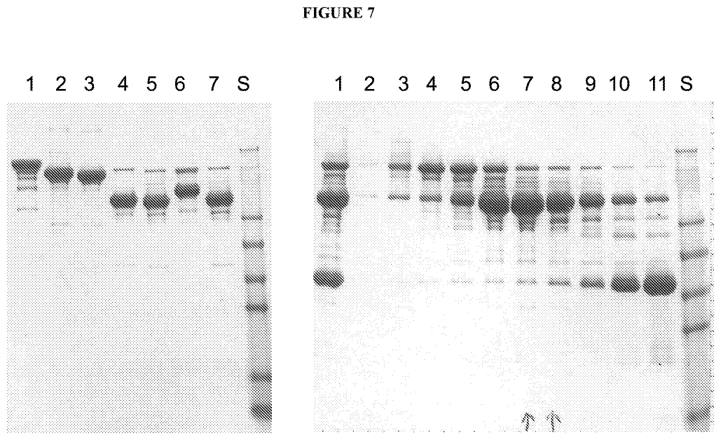

FIG. 7 (left panel) shows an SDS-PAGE analysis under non-reducing conditions of the 6 other products described in Table 1. Versions 3438, 3440, 3441 contain the characteristic HC2LC2 dimeric structure characteristic of antibodies and were >95% pure following Protein A purification. In contrast, versions 3442, 3443, and 3444 after protein A purification were very heterogeneous. They contained the expected mixture of components one would anticipate for heteromer designs reflecting the 3 different possibilities of generating the desired heteromer and homomers of either arm. Size exclusion chromatography (SEC) was used to enrich the heteromers from the mixture. For SEC fractionation, the protein A eluates were concentrated and then subjected to SEC on a 24 mL Superdex 200 column at a flow rate of 0.5 mL/min in 20 mM Na₂HPO₄ pH 7.4, 150 mM NaCl. Column fractions were collected, analyzed by SDS-PAGE, and those containing heteromers were pooled, filtered, aliquoted, and stored at −70° C. FIGS. 7 (right panel) shows SEC fractionation of sample 3443. Fractions shown in lanes 7 and 8 (denoted with arrows) were pooled to generate the sample shown in the left panel in lane 7.

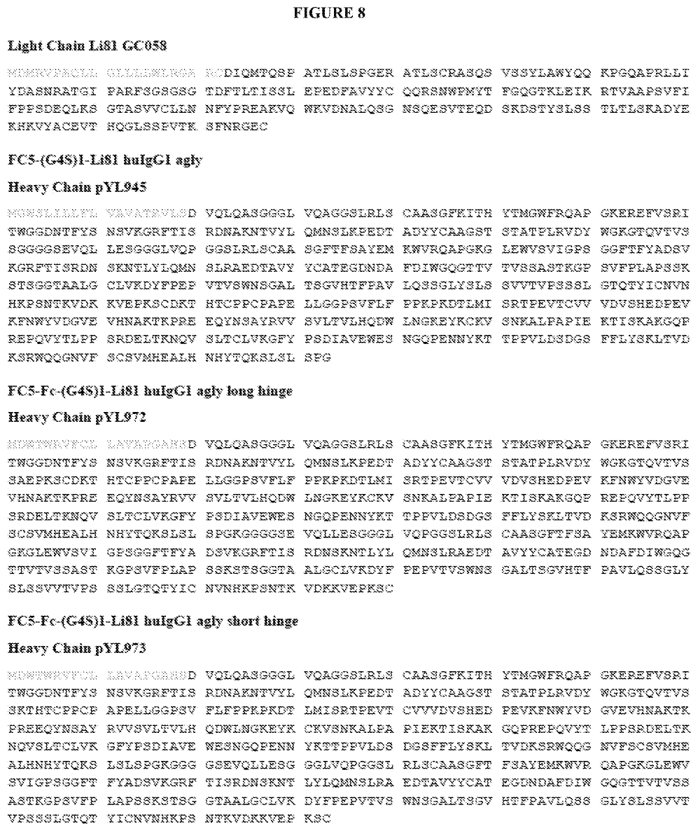

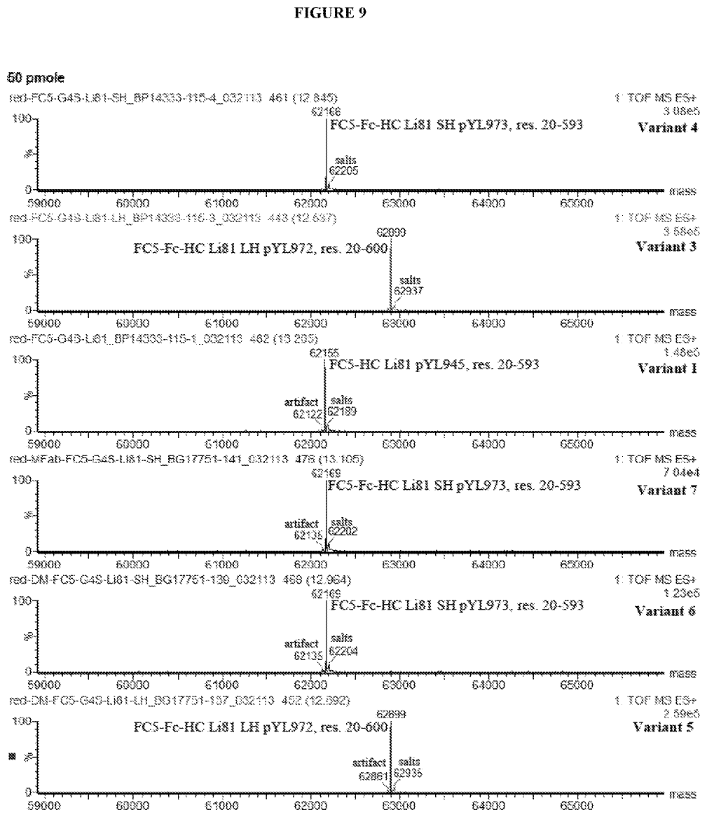

FIG. 8 shows the amino acid sequence of heavy and light chains for the various FC5-(G₄S)1-Li81 constructs. Intact mass analysis of the reduced heavy chain 3438 (predicted mass 62,153.9 Da, observed mass 62155 Da), reduced heavy chain 3440 (predicted mass 62,897.8 Da, observed mass 62899 Da), reduced heavy chain 3441 (predicted mass 62,167.0 Da, observed mass 62168 Da), reduced heavy chain 3442 (predicted mass 62,897.8 Da observed mass 62899 Da), reduced heavy chain 3443 (predicted mass 62,167.0 Da, observed mass 62169 Da), reduced heavy chain 3444 (predicted mass 62,167.0 Da, observed mass 62169 Da). All of the constructs contained the Li81 light chain (reduced light chain predicted mass 23,628.4, observed mass 23629 Da). These analyses showed that the major components in all samples contained the predicted light chain and heavy chain. The corresponding deconvoluted spectra for the heavy chains are shown in FIG. 9. No unexpected modifications on the major component were observed.

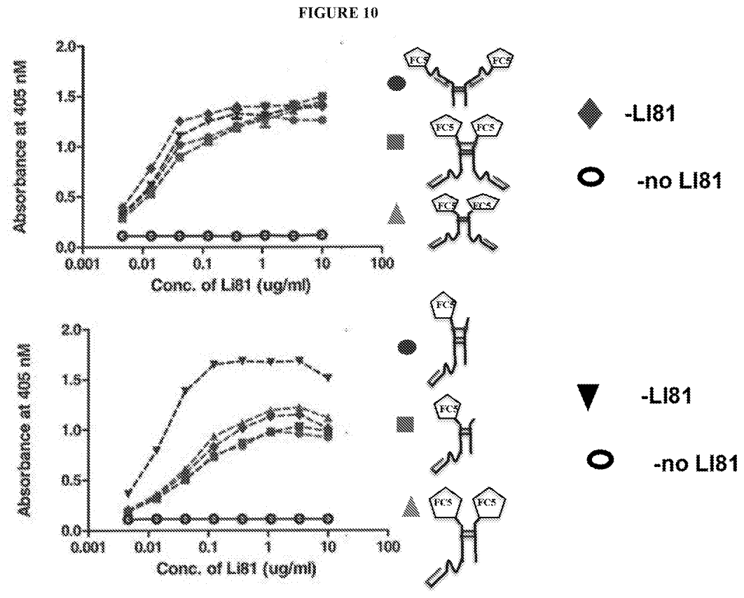

To test if the FC5 fusions impacted Li81 function, samples were analyzed for binding to LINGO-1 in an ELISA format in which 96 well plates were coated with LINGO-1, then treated with serial dilutions of the FC5-Li81 bispecifics. FIG. 10 shows the ELISA data for the 6 FC5-(G₄S)1-Li81 constructs. The 3 bivalent constructs (3438, 3440, 3441) were all equipotent with Li81 without FC5 attached, indicating that the addition of the FC5 did not impact the binding of the constructs to LINGO-1. In contrast the 3 heteromeric versions (3442, 3443, and 3444), containing a single copy of the Li81 Fab showed a reduced signal, consistent with the presence of one versus two binding sites present in the bivalent constructs. The three FC5-(G₄S)3-Li81 constructs were also tested in the ELISA and were equipotent with Li81 without FC5 attached. The 9 samples were also tested for function in an oligodendrocyte differentiation assay and all were active. The EC50 for promoting differentiation in the potency assay were all about 3-fold lower that Li81 without FC5 attached. Samples were characterized for activity by ELISA and in the differentiation assay using the methods previously described for characterization of the Li81 antibody (Pepinsky et al., Journal of Pharmacology and Experimental Therapeutics, 339:519-529 (2011)).

The biological activity of the FC5 moiety of the FC5-Li81 samples was assessed in an in vitro BBB transwell cell culture assay on Simian virus 40 immortalized-adult rat brain endothelial cells (SV-ARBEC). The molecules were co-applied to the upper chamber of the in vitro BBB model in paired combinations (control and test molecule) and quantified by mass spectrometry quantitation (MRM) in the bottom compartment; P_{APP} value for each was calculated over 90 min. The (top chamber) input concentration of FC5-Li81 was between 1.5 and 3 μM (linear phase) with equimolar input of various co-administered control antibodies. Results from this analysis are shown in FIG. 11. All of the samples showed increased FC5-mediated transport relative to the controls with improvements in P_{APP} values ranging from 2-15 fold. The 3 bivalent FC5-(G₄S)3-Li81 constructs (3014, 3015, 3016) were all equipotent, indicating that whether the addition of the FC5 was on the HC, LC, or both did not impact the activity of the constructs. All showed about a 3-fold improvement in P_{APP} value. Most significant was the fold improvement seen with FC5-(G₄S)1-Li81 (3438), as this version showed about a 15-fold increase in its P_{APP} value, ˜5-fold greater than the corresponding FC5-(G₄S)3-Li81 (3015). The FC5-(G₄S)3-Li81 (3015) and FC5-(G₄S)1-Li81 (3438) constructs only differ in the length of the G₄S sequence (SEQ ID NO:5) connecting FC5 with Li81 as both contain the FC5 fused to the Li81 HC. FC5-(G₄S)1-Li81 (3440) construct in which FC5 was fused to the Fc and the Li81 Fab was attached to the C-terminus of the Fc through a (G₄S)1 linker also showed a large ˜12-fold improvement in its P_{APP} value. In contrast, when the same construct design with a shorter but more flexible hinge (3441) was tested it showed only about a 6-fold improvement in its P_{APP} value. Thus similar to what was seen with the linker length that connects constructs 3015 and 3438, the design of the hinge in constructs 3440 and 3441 impacted the in vitro transport activity of the constructs. The 3 heteromeric versions (3442, 3443, and 3444), showed improvements relative the control ranging from 2-5 fold. While the increases in transport were significant, these constructs were not tested in in vivo studies.

The in vitro transwell assay was performed as follows. SV-ARBECs were seeded at 80,000 cells/membrane on rat-tail collagen coated 0.83 cm² Falcon cell inserts, 1 μm pore size in 1 mL SV-ARBEC feeding media without phenol red. The wells of a 12-well tissue culture plate (i.e., bottom chamber for transport) contained 2 mL of 50:50 (v/v) mixture of SV-ARBEC media without phenol red and rat astrocyte-conditioned media. The model characterization is described in detail in Garberg et al., Toxicol In Vitro, 19(3):299-334 (2005). The model was used when Pe[sucrose] was between 0.4-0.6 [×10⁻³] cm/min. Transport experiments were performed as described in Haqqani et al., Mol. Pharm., 10:1542-1556 (2012) by adding a mixture of test sample and control in equimolar concentrations to the top chamber and by collecting the 100 μl aliquots (with subsequent replacement with 100 μl of transport buffer) from the bottom chamber at 15, 30, 60 and 90 min for simultaneous quantification of all antibodies using multiplexed SRM-ILIS method. The apparent permeability coefficient Papp was calculated as described previously (Haqqani et al., 2012 (supra)).

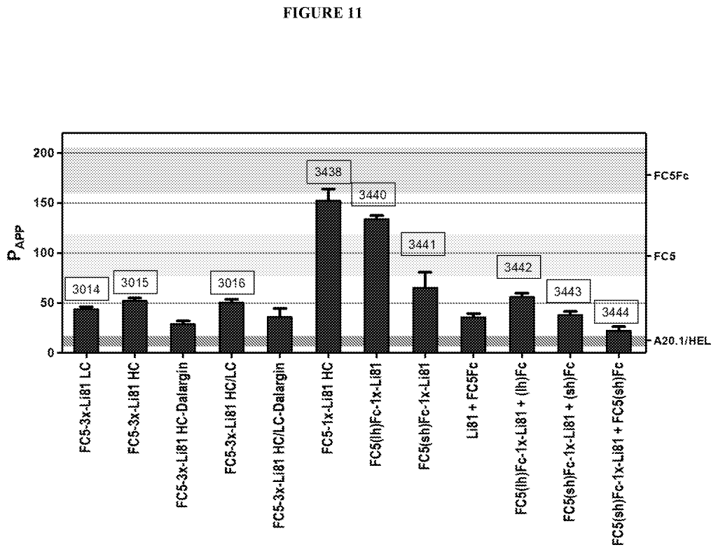

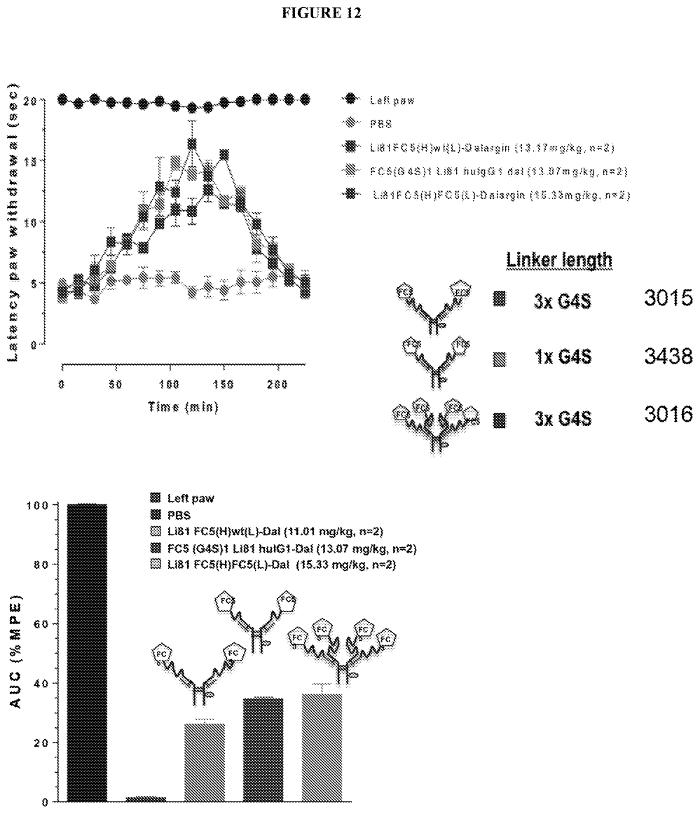

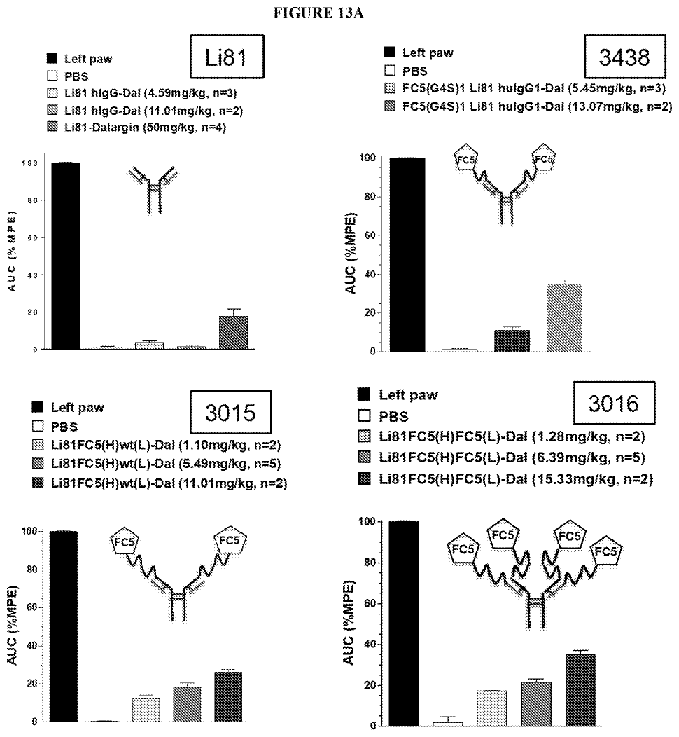

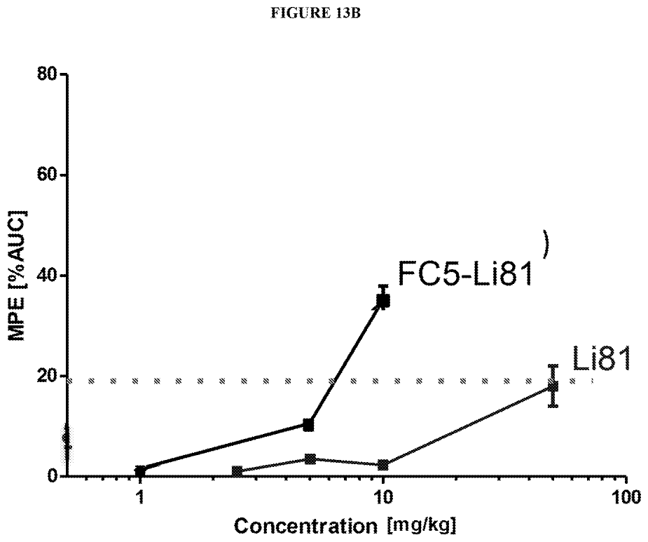

To test if the FC5-Li81 constructs showed BBB transport activity in vivo, constructs 3015, 3438, and 3440 were tested in the Hargreaves model of inflammatory hyperalgesia. In this model the constructs were chemically conjugated to dalargin, administered by intravenous (IV) injection, and pain response measured overtime. The methodology for running the model is as previously described (Farrington et al, A novel platform for engineering blood-brain barrier-crossing bispecific biologics, FASEB J., 28(11):4764-78 (2014)). Opioid peptide dalargin does not cross the BBB and is not analgesic after systemic dosing. As shown in FIG. 12, systemic dosing of the FC5-Li81 Dalargin conjugates induced a significant analgesic response. The data show that dalargin chemically linked to FC5-Li81 can engage central opioid receptors after systemic administration. AUC values for the three treatments were similar ranging from 25-35% of the maximum possible effect (MPE). FIG. 13A shows the dose dependence of the responses of the three constructs and Li81 as in control in the model. FIG. 13B is a plot of construct 3438 and Li81. Treatment of FC5-Li81 lead to a 20% MPE as a % of area under the curve (AUC) occurred at 10-fold lower dose than for Li81 alone, thus indicating the addition of FC5 to the Li81 improved its ability to cross the blood brain barrier and engage opioid receptors.

The FC5-Li81 fusions and Li81 alone were conjugated to dalargin analog Tyr-dAla-Gly-Phe-Leu-Arg-cysteamide (Dal-Cys) using the general method as described by Mattson et al., Mol. Biol. Rep., 17:167-183 (1993). Briefly sulfo-succinimidyl-44N-maleimidomethyl]cyclohexane-1-carboxylate (SMCC; Pierce Rockland, Ill., USA) at 10 mg/mL in N,N-dimethylformamide was added at a 7.5-fold molar excess to the test antibody at 1-2 mg/mL in PBS. The reaction was incubated for 2.5 h at room temperature, and Dal-Cys in 50 mM MES (pH 6.0) was added in a 4-fold molar excess to the SMCC. The Dal-labeled protein was concentrated in a centrifugal device and desalted to remove unconjugated Dal-Cys and SMCC. The stoichiometry of Dalargins/molecule was determined from intact mass spectrometry analysis of labeled proteins. FIG. 11 shows the transport activity of Dalargin conjugates of 3015 and 3016. Under these conditions the conjugation resulted in a 20-40% decrease in P_{APP} values relative to the corresponding proteins without dalargin added.

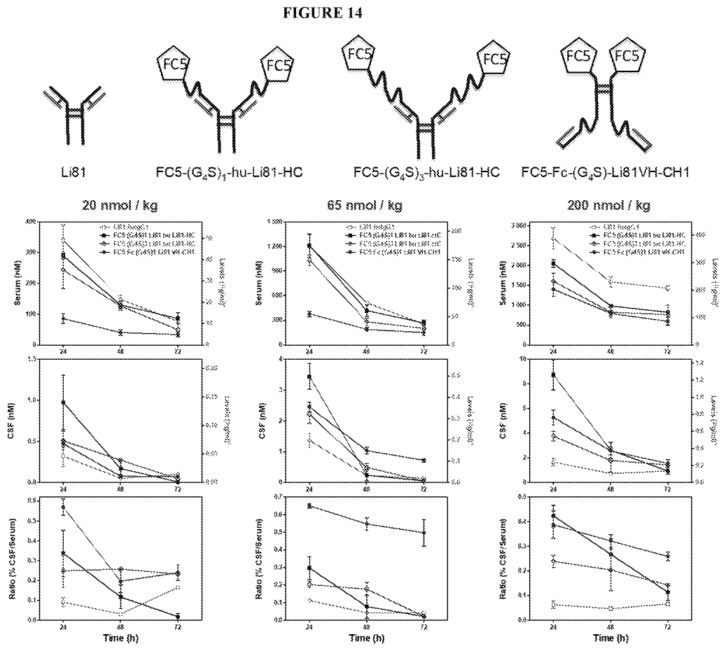

While the assessment of dalargin conjugates of Li81 and the FC5-Li81 constructs provided clear in vivo evidence that fusions an antibody to FC5 can improve its delivery into the central nervous system (CNS), the duration of the opioid readout lasted only 4 hours. In order to obtain a more quantitative readout of the transporter and explore the duration of the effect, we performed a pharmacokinetic analysis assessing serum and CNS levels by mass spectrometry at 24, 48, and 72 h post intravenous administration. FIG. 14 shows results from the analysis of Li81 and three FC5-Li81 constructs (3015, 3438, and 3441) at doses of 20, 65, and 200 nmol/kg. All of the FC5 fusions at all concentrations led to elevated CSF levels at 24 hours relative to levels observed with Li81 mAb and there was a clear dependence of the CSF levels with administered dose. In contrast, at the 72 h time point the difference from the control was diminished. Consistent with this, the CSF/serum ratios for the FC5 fusions decreased over time, while Li81 CSF/serum ratios were relatively constant at all time points and at all doses. These data verify that FC5 improves delivery of Li81 to the CNS, but the effect is reduced over time.

### Example 2: Production of FC5-(G₄S)1-Li81

FC5-(G₄S)1-Li81
Light Chain GC058
(SEQ ID NO: 2)
MDMRVPAQLL GLLLLWLRGA RCDIQMTQSP
ATLSLSPGER ATLSCRASQS VSSYLAWYQQ
KPGQAPRLLI YDASNRATGI PARFSGSGSG
TDFTLTISSL EPEDFAVYYC QQRSNWPMYT
FGQGTKLEIK RTVAAPSVFI FPPSDEQLKS
GTASVVCLLN NFYPREAKVQ WKVDNALQSG
NSQESVTEQD SKDSTYSLSS TLTLSKADYE
KHKVYACEVT HQGLSSPVTK SFNRGEC
Heavy Chain pYL945
(SEQ ID NO: 7)
MGWSLILLFL VAVATRVLSD VQLQASGGGL
VQAGGSLRLS CAASGFKITH YTMGWFRQAP
GKEREFVSRI TWGGDNTFYS NSVKGRFTIS
RDNAKNTVYL QMNSLKPEDT ADYYCAAGST
STATPLRVDY WGKGTQVTVS SGGGGSEVQL
LESGGGLVQP GGSLRLSCAA SGFTFSAYEM
KWVRQAPGKG LEWVSVIGPS GGFTFYADSV
KGRFTISRDN SKNTLYLQMN SLRAEDTAVY
YCATEGDNDA FDIWGQGTTV TVSSASTKGP
SVFPLAPSSK STSGGTAALG CLVKDYFPEP
VTVSWNSGAL TSGVHTFPAV LQSSGLYSLS
SVVTVPSSSL GTQTYICNVN
HKPSNTKVDK KVEPKSCDKT HTCPPCPAPE
LLGGPSVFLF PPKPKDTLMI SRTPEVTCVV
VDVSHEDPEV KFNWYVDGVE VHNAKTKPRE
EQYNSAYRVV SVLTVLHQDW LNGKEYKCKV
SNKALPAPIE KTISKAKGQP REPQVYTLPP
SRDELTKNQV SLTCLVKGFY PSDIAVEWES
NGQPENNYKT TPPVLDSDGS FFLYSKLTVD
KSRWQQGNVF SCSVMHEALH NHYTQKSLSL
SPG

FC5-(G₄S)1-Li81 was expressed in CHO cells. For preparation of the conditioned medium, transfected CHO cells from an unsorted pool were expanded in serum-free media, grown to high density, fed with supplements, and shifted to a reduced temperature. Cultures were held at this reduced temperature for 14 days and then harvested by centrifugation and clarified by 0.45-micron filtration. 1.8 L of clarified and filtered culture medium (estimated titer of FC5-Li81 of 150 mg/L) was loaded onto a 20 mL protein A column. The column was washed with 10 mM Na₂HPO₄ pH 7.0, 150 mM NaCl and FC5-(G₄S)1-Li81 was eluted from the column with 25 mM Na₂HPO₄ pH 2.8, 100 mM NaCl pH 2.8 and neutralized with 25 mM NaH₂PO₄ pH 8.6 diluted from a 0.5 M stock solution. The protein content of the eluted samples was estimated from the absorbance at 280 nm using an extinction coefficient of 1.4 for a 1 mg/mL solution. 290 mg of purified FC5-(G₄S)1-Li81 was recovered. The preparation was filtered, aliquoted, and stored at −70° C.

### Example 3: Assessing the Stability of FC5 (G₄S)1-Li81 in Rat Serum

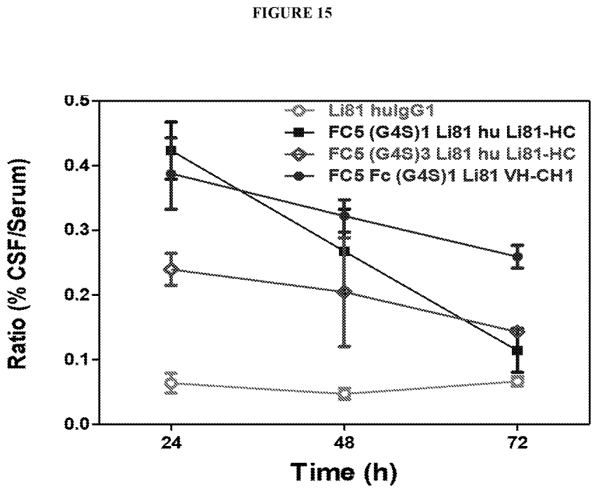

A pharmacokinetic analysis of FC5-(G₄S)1-Li81 in rats following intravenous dosing at 30 mg/Kg and measuring serum and CSF levels at 24, 48, and 72 h, revealed that the transporter activity of FC5 decreased over time where it was highest in CSF at the earliest time point tested 24 h with a CSF/serum ratio of 0.45%, decreased to a CSF/serum ratio of 0.28% at 48 h, and further decreased to a CSF/serum ratio of 0.13% after 72 h. Results from this analysis are presented in FIG. 15. Serum levels of FC5-(G₄S)1-Li81 at the 72 h time point were ˜150 μg/mL and therefore did not account for the reduction of protein detected in the CSF.

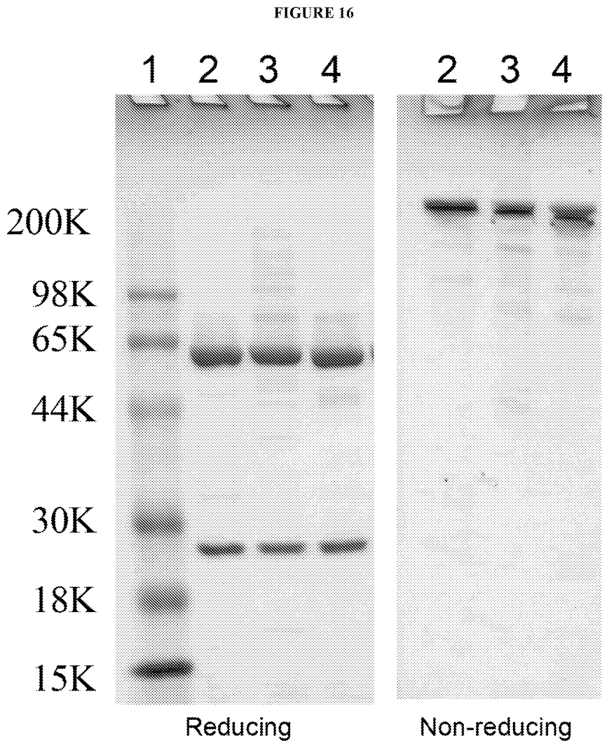

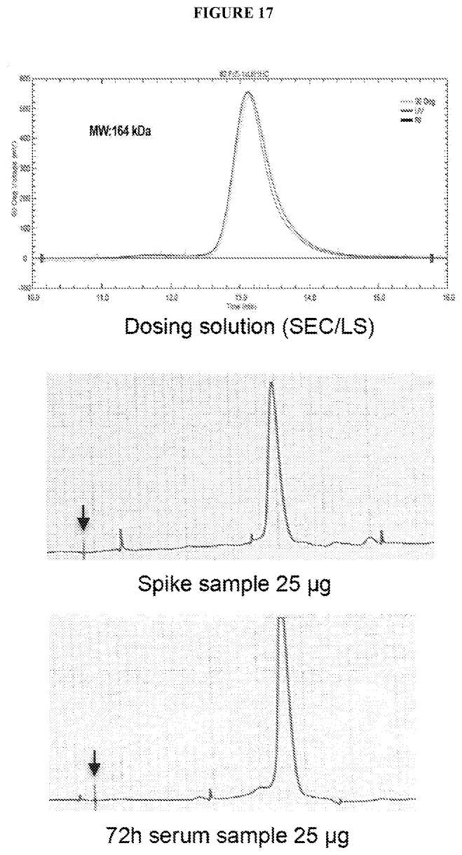

To determine if loss of transport activity resulted from a change in the biochemical or functional properties of FC5-(G₄S)1-Li81, three rats were dosed intravenous with 30 mg/Kg FC5-(G₄S)1-Li81 and the protein was purified from the serum after 72 h and characterized as follows. After 72 h, the dosed animals were exsanguinated and serum was prepared. The serum was filtered through a 0.45 μsyringe driven filter unit. 12 mL of pooled serum from the 3 rats was batch loaded for 1.5 h onto 350 μL Ig-Select (GE Healthcare). The resin was collected in a column, washed with 12 column volumes of 20 mM Na₂HPO₄ pH 7.0, 150 mM NaCl (PBS), 12 column volumes of 25 mM NaH₂PO₄ pH 5.5, 100 mM NaCl, and then eluted with 25 mM NaH₂PO₄ pH 2.8, 100 mM NaCl pH 2.8. 200 μL fractions were collected. The protein content of the eluted samples was estimated from the absorbance at 280 nm using an extinction coefficient of 1.4 for a 1 mg/mL solution (fractions 4-7 pooled and neutralized with 25 mM NaH₂PO₄ pH 8.6 diluted from a 0.5 M stock solution). The elution pool 800 μL had a concentration of 1.9 mg/mL. Recoveries were >90% based on the measured concentrations of FC5-(G₄S)1-Li81 in the serum as determined by MRM. The sample was aliquoted, flash frozen on dry ice, and stored at −70° C. As a control for sample processing, 1.2 mg of FC5-(G₄S)1-Li81 was spiked into 10 mL of Wistar rat serum (Innovative Research, Inc.) and subjected to the sample purification process as the sample that was recovered from the in vivo study. FIG. 16 shows an analysis of the purified samples by SDS-PAGE under reducing and non-reducing conditions. The FC5-Li81 heavy chain (HC) 60 kDa and light chain (LC) 25 kDa bands were observed under reducing conditions and characteristic 175 kDa tetrameric 2FC5Li81HC-2LC complex was observed under non-reducing conditions. The Li81 samples showed the expected size exclusion chromatography (SEC) elution profiles (FIG. 17) eluting as single prominent peaks with molecular masses of 165 kDa.

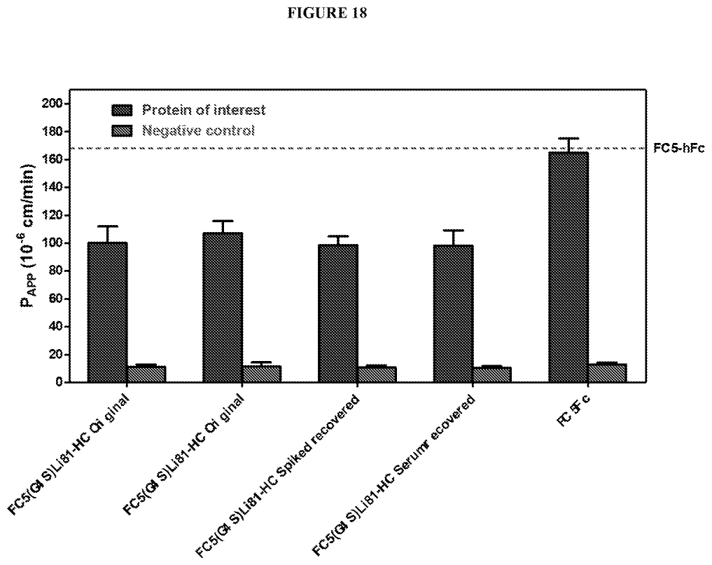

The biological activity of the FC5-(G₄S)1-Li81 samples were assessed in an in vitro BBB transwell cell culture assay on SV-ARBEC cells. The molecules were co-applied to the upper chamber of the in vitro BBB model in various paired combinations (control and test molecule) and quantified by MRM in the bottom compartment; Papp value for each was calculated over 90 min. The (top chamber) input concentration of FC5-Li81 samples was between 1.5 and 3 μM (linear phase) with equimolar input of various co-administered control antibodies. Results from this analysis are shown in FIG. 18. Transport activity of FC5-(G₄S)1-Li81 was not significantly altered after 72h in rats.

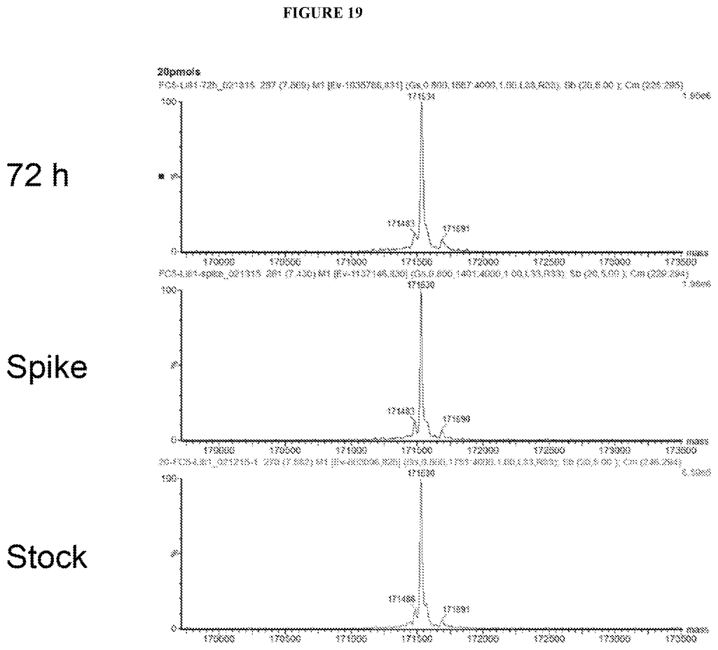

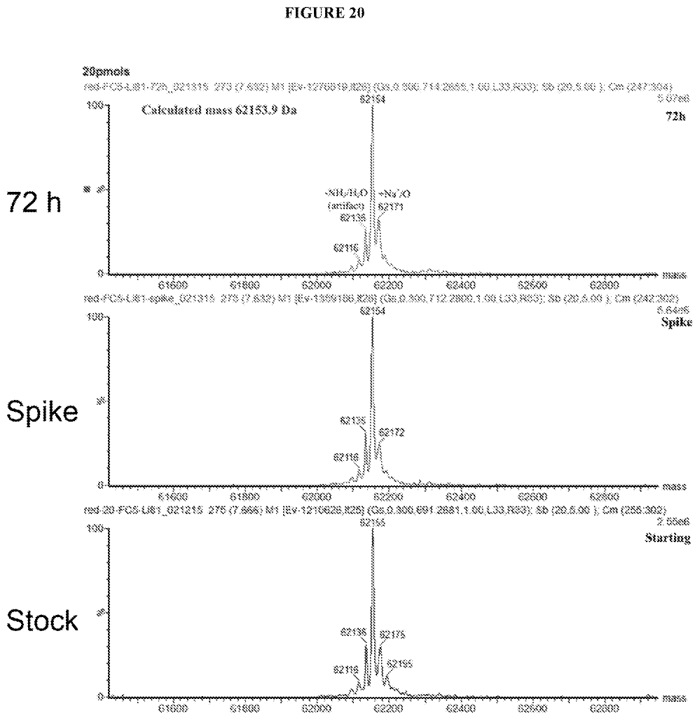

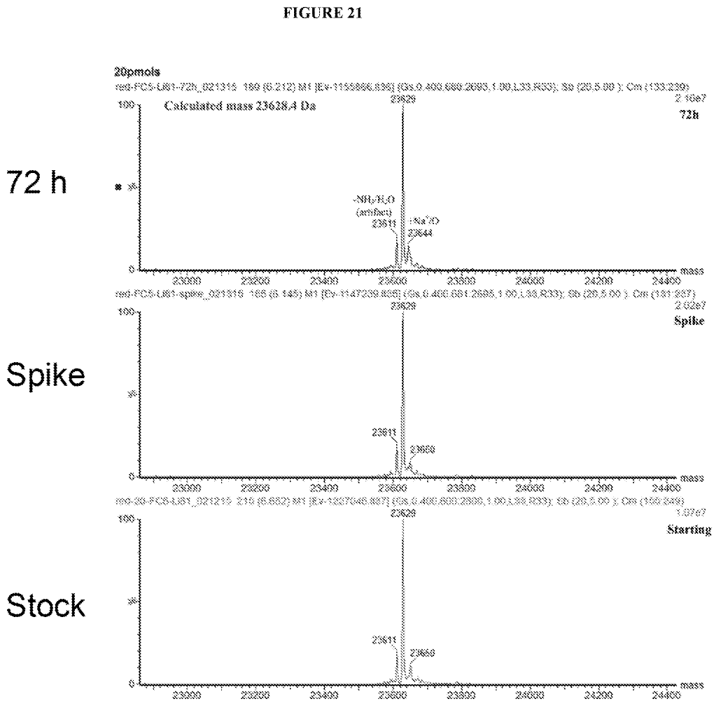

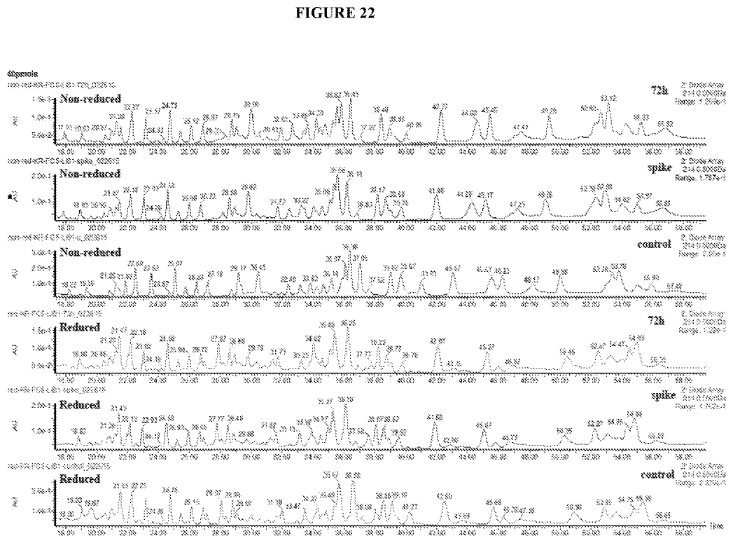

The FC5-(G₄S)1-Li81 samples were subjected to extensive characterization by mass spectrometry. First, intact mass analysis under both reducing and non-reducing conditions showed that the major component in all samples was the predicted protein, FC5-(G₄S)1-Li81 (predicted mass non reduced 171,528.3 Da; observed mass dosing solution 171530 Da, spiked sample 171530 Da, 72 h serum sample 171534 Da: predicted mass reduced FC5Li81 HC 62,153.9 Da; observed mass dosing solution 62155 Da, spiked sample 62154 Da, 72 h serum sample 62154 Da: predicted mass reduced LC 23,628.4 Da; observed mass dosing solution 23629 Da, spiked sample 23629 Da, 72 h serum sample 23629 Da). Results from these analyses are shown in FIGS. 19, 20, and 21. No unexpected modifications on the major component were observed. For these analysis about 20 pmol of each sample was analyzed under non-reducing and reducing (40 mM DTT in 4 M urea and 10 mM EDTA at 37° C. for 1 h) conditions on the Xevo G2 mass spectrometer using a MassPREP Micro Desalting 20 μm column (2.1 id×5 mm Phenyl, Waters) for separation. The molecular masses were generated by deconvolution using the MaxEnt 1 program. Second, the disulfide structure for all the cysteines were determined by peptide mapping with LC/MS detection. FIG. 22 shows reduced and non-reduced peptide maps for the 3 test samples. Analysis of disulfide containing peptides showed that the major disulfide linkages are all as expected for each sample (Table 2).

TABLE 2
List of major disulfide linked peptides in FC5(G₄S)1Li81

| | | Calculated | | | | | | | |
|---|---|---|---|---|---|---|---|---|---|
| | | Peptide | RT | | Charge | Mass | Intensity (Counts) | | |
| Peptide | #Cys | Mass (Da) | (Min) | m/z | State | (Da) | control | spike | 72 h |
| LC19-24═LC62-91 | C1═C2 | 3951.793 | 50.0 | 1118.278 | 3 | 3951.810 | 2419334 | 1699454 | 1256865 |
| LC128-143═LC192-208 | C3═C4 | 3555.749 | 45.5 | 1186.263 | 3 | 3555.765 | 4302261 | 2820901 | 2137050 |
| LC209-215═HC347-350 | C5═C7 | 1265.486 | 18.2 | 631.254 | 2 | 1260.492 | 188979 | 114375 | 114513 |
| LC209-215═HC347-350 | C5═C7 | 1292.458 | 19.4 | 647.241 | 2 | 1292.466 | 6948 | 3439 | N/D |
| | (trisulfide) | | | | | | ~4.4% | ~4.3% | |
| HC20-28═HC77-108 | C1═C2 | 4361.029 | 36.4 | 3454.693 | 3 | 4361.056 | 1779579 | 1013912 | 493717 |
| HC147-162═HC215-249 | C3═C4 | 5382.348 | 48.2 | 1795.132 | 3 | 5382.371 | 682418 | 328427 | 185398 |
| HC262-275═HC276-338 | C5═C6 | 7916.919 | 53.8 | 1594.397 | 5 | 7916.944 | 2073621 | 1556210 | 1178534 |
| HC351-376═HC351-376 | C8═C8, | 5454.783 | 53.1 | 1364.709 | 4 | 5454.806 | 1205007 | 818185 | 721037 |
| | C9═C9 | | | | | | | | |
| HC351-376═HC351-376 | C8═C8/C9═C9 | 5486.755 | 52.5 | 1372.697 | 4 | 5486.757 | *1.1% | 1288 | N/D |
| | (trisulfide) | | | | | | | ~1.2% | |
| HC351-376═HC351-374 | C8═C8, | 5229.036 | 55.2 | 1308.422 | 4 | 5229.654 | 1033732 | 679782 | 568233 |
| | C9═C9 | | | | | | | | |
| HC384-402═HC449-450 | C10═C11 | 2328.098 | 30.5 | 777.044 | 3 | 2328.198 | 2859382 | 1817917 | 1267722 |
| HC489-498═HC543-567 | C12═C13 | 4087.957 | 35.4 | 1023.003 | 4 | 4087.982 | 100423 | 98857 | 110635 |
| HC489-498═HC545-567 | C12═C13 | 3844.824 | 36.4 | 1282.623 | 3 | 3844.844 | 1578858 | 1150987 | 589348 |

*Amount calculated from EIC

Notably the presence of the heavy chain C1-C2 disulfide indicates that the FC5 domain is properly folded in the fusion protein. Analysis showed that the trisulfide level, based on peak area from extracted ion chromatograms (EICs), was ˜4.5% of the linkage between the heavy chain (Cys348) and light chain (Cys215) and −1% in the hinge of the heavy chain in control and spike samples. Trisulfides were not observed in the 72 h sample, consistent with the previously published trisulfide data for Li81 (mAb1-B in Gu et al. 2010. Anal Biochem. 400(1):89-98). The methods used for disulfide peptide mapping of the FC5-(G₄S)1-Li81 samples are as follows. About 1.4 μL of 1:10 diluted 4-vinylpyridine [in 8 M guanidine hydrochloride was added to a solution containing ˜24 μg (240 pmol, non-reduced) of protein, immediately followed by adding 60 μL of 8M guanidine HCl (final volume was ˜70μ). The solution was held at room temperature in the dark for 45 min. Protein in each vial was recovered by precipitation with 40 volumes of cooled ethanol. The mixture was stored at −20° C. for 1 h and then centrifuged at 14000 g for 12 min at 4° C. The supernatant was discarded and the precipitate was washed once with cooled ethanol. The protein pellet was re-dissolved with 100 μL 0.1% trifluoroacetic acid in 50% acetonitrile and dried in a Speed-Vac for 1.5 h. Two different peptide mapping methods were used to analyze the samples; one utilizing endoproteinase Lys-C and trypsin, and the other with endoproteinase AspN. For the LysC/trypsin maps, about 8 μg of the 4-vinylpyridine-treated protein in each sample was digested with 5% (w/w) endoproteinase Lys-C(Wako) in 2 M urea, 0.15 M Tris-HCl, pH 6.7, 2 mM CaCl₂, 5 mM methylamine, at room temperature for 5 h, then 5% (w/w) trypsin (Promega) was added. The digest was held for an additional 4 h at room temperature followed by the addition of 5% (w/w) trypsin and then the digest was held at room temperature overnight. The total volume was 50 μL. For digestions with AspN, about 4 μg of the 4-vinylpyridine-treated protein was digested with 5% (w/w) Asp-N(Roche) in 2 M urea, 0.15 M Tris-HCl, pH 6.7, 5 mM MgCl₂, 5 mM methylamine, at room temperature for 8 h, then 5% (w/w) Asp-N was added and the digest was held overnight. The total volume was 50 μL. Prior to analysis of the digests by LC-MS, each digest was quenched by adding 1 μL of 25% trifluoroacetic acid and 4 M urea. Half of the Lys-C/tryptic digest and all of Asp-N digest were reduced with 100 mM TCEP in 1 mM EDTA at room temperature for 1 h. An aliquot (˜4 μg) of the non-reduced and reduced Lys-C/tryptic digests and the reduced Asp-N digests was analyzed on an LC-MS system composed of a UPLC-Xevo G2-S system. The column and separation gradient were the same as described in the peptide mapping section. Disulfide mapping data were processed using BioPharmaLynx 1.3.3.

The reduced Lys-C/tryptic and Asp-N maps of 4-vinylpyridine-treated, non-reduced samples were examined to evaluate free thiol levels and to assess if there were any significant difference in the level of free thiols in the control, spike, and 72 h samples. The amounts of free thiols were estimated from extracted ion chromatograms (EIC). The analysis revealed small amounts of free thiols in all samples, but none in the FC5 region. Free thiol levels were 2-3 folds higher in the 72 h sample than that in the control and spike samples.

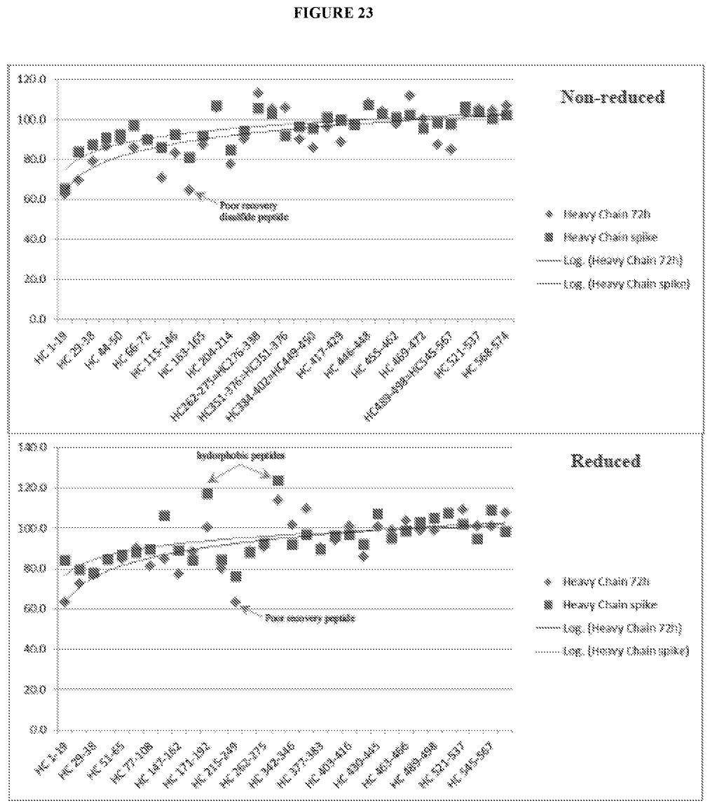

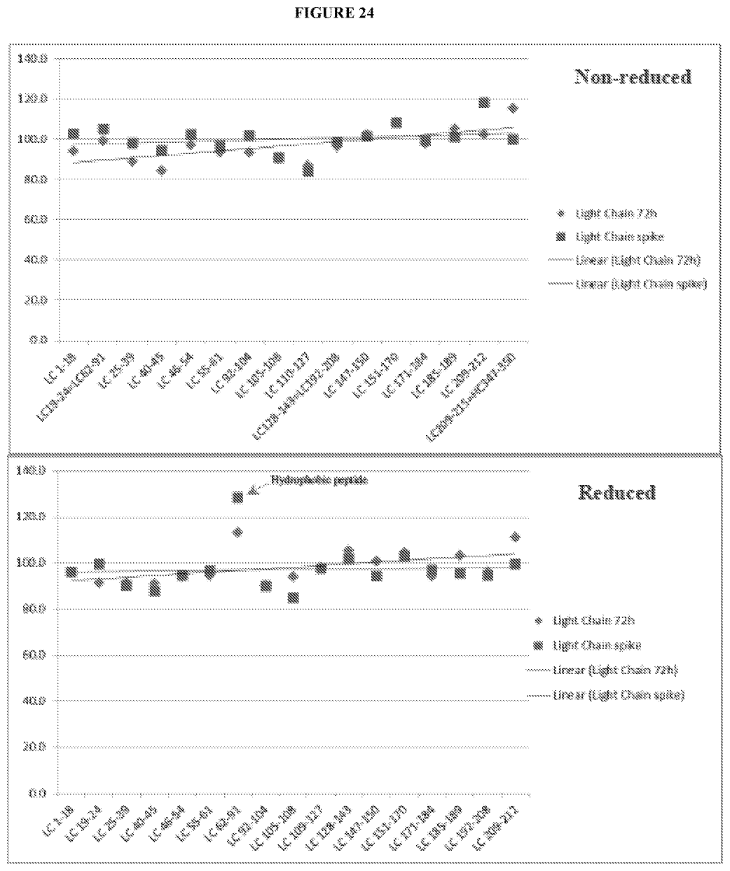

Systematic comparison of the peptide recoveries in the peptide maps of the 72 h and spike samples with those in the maps of the control sample without exposure to rat serum indicated that N-terminal clipping of the FC5-Li81 heavy chain occurred in serum because the peptide recovery gradually decreased starting from the end of the HC variable region (peptide HC 215-249) to the N-terminus (peptide HC 1-19) (FIG. 22). Recovery of the N-terminal peptide (HC residues 1-19) was 60%, indicating that 40% of the protein was lacking this peptide. Recovery of the next peptide (HC29-38) was 70% and recovery of peptide 51-65 was 80%. In contrast recovery of peptides from within the Li81 HC were ˜100%, indicating that clipping was largely restricted to within the FC5 portion of the fusion protein. N-terminal clipping was detected in the serum spike sample; however, the extent of clipping was much more extensive in the 72 h sample than the spike sample (FIG. 23). In contrast, recovery of light chain peptides was near 100% throughout the sequence for all the samples (FIG. 24). These findings indicate that the FC5 portion of the fusion protein is labile in serum and that proteolysis at multiple sites within the FC5 domain leads to poor recovery of the N-terminal peptide.

### Example 4: Humanization of FC5

To reduce the risk of immunogenicity of FC5 in humans, while maintaining solubility and stability, a series of FC5 sequence variants with varying degrees of homology to the human VH3 consensus framework were designed. For all variants, the following nine humanization mutations were included: Q5V, A6E, A14P, A75S, K87R, P88A, D93V, K114Q, and Q117L. Mutations in the remaining six framework positions that distinguish different humanization designs are listed in Table 3. These include mutations at the corresponding human IgG VH/VL interface (amino acid positions 37, 44, 45, and 47) as well as at amino acid positions 1 and 79. Partial humanization of the corresponding interface positions is probed in mutants H31 and H32. Lastly, both the camelid FC5 wild-type and the human VH3 consensus framework contain a methionine in CDR-H1 (M34) that may be partially solvent exposed, based on a computational model of FC5 (see below). Exposed methionine can be a liability for developability due to their potential to be modified by oxidation. Thus, each humanization design was produced with and without a M34T substitution, as indicated in Table 3. In the odd numbered constructs, the methionine at position 34 is retained and in the even numbered constructs the methionine is replaced with a threonine.

TABLE 3
List of Amino Acid Substitutions Distinguishing
FC5 Humanization Designs

| Position | 1 | 34 | 37 | 44 | 45 | 47 | 79 |
|---|---|---|---|---|---|---|---|
| Camelid | D | M | F | E | R | F | V |
| H11 | E | M | V | G | L | W | L |
| H12 | E | T | V | G | L | W | L |
| H31 | E | M | F | G | L | F | V |
| H32 | E | T | F | G | L | F | V |
| H61 | D | M | F | E | R | F | V |
| H62 | D | T | F | E | R | F | V |
| H71 | E | M | F | E | R | F | L |
| H72 | E | T | F | E | R | F | L |

Table 4 provides the full VHH sequences from the eight humanized FC5 variant designs listed in Table 3 (residue substitutions in bold), as well as sequences for the short- and long-hinge hIgG1-Fc agly designs.

TABLE 4
Humanized FC5 Variants

| SEQ | | |
|---|---|---|
| ID | Humanized | |
| NO. | Variant | Amino Acid Sequence |
| 10 | H11 | EVQLVESGGGLVQPGGSLRL |
| | | SCAASGFKITHYTMGWVRQA |
| | | PGKGLEWVSRITWGGDNTFY |
| | | SNSVKGRFTISRDNSKNTLY |
| | | LQMNSLRAEDTAVYYCAAGS |
| | | TSTATPLRVDYWGQGTLVTV |
| | | SS |
| 11 | H12 | EVQLVESGGGLVQPGGSLRL |
| | | SCAASGFKITHYTTGWVRQA |
| | | PGKGLEWVSRITWGGDNTFY |
| | | SNSVKGRFTISRDNSKNTLY |
| | | LQMNSLRAEDTAVYYCAAGS |
| | | TSTATPLRVDYWGQGTLVTV |
| | | SS |
| 12 | H31 | EVQLVESGGGLVQPGGSLRL |
| | | SCAASGFKITHYTMGWFRQA |
| | | PGKGLEFVSRITWGGDNTFY |
| | | SNSVKGRFTISRDNSKNTVY |
| | | LQMNSLRAEDTAVYYCAAGS |
| | | TSTATPLRVDYWGQGTLVTV |
| | | SS |
| 13 | H32 | EVQLVESGGGLVQPGGSLRL |
| | | SCAASGFKITHYTTGWFRQA |
| | | PGKGLEFVSRITWGGDNTFY |
| | | SNSVKGRFTISRDNSKNTVY |
| | | LQMNSLRAEDTAVYYCAAGS |
| | | TSTATPLRVDYWGQGTLVTV |
| | | SS |
| 14 | H61 | DVQLVESGGGLVQPGGSLRL |
| | | SCAASGFKITHYTMGWFRQA |
| | | PGKEREFVSRITWGGDNTFY |
| | | SNSVKGRFTISRDNSKNTVY |
| | | LQMNSLRAEDTAVYYCAAGS |
| | | TSTATPLRVDYWGQGTLVTV |
| | | SS |
| 15 | H62 | DVQLVESGGGLVQPGGSLRL |
| | | SCAASGFKITHYTTGWFRQA |
| | | PGKEREFVSRITWGGDNTFY |
| | | SNSVKGRFTISRDNSKNTVY |
| | | LQMNSLRAEDTAVYYCAAGS |
| | | TSTATPLRVDYWGQGTLVTV |
| | | SS |
| 16 | H71 | EVQLVESGGGLVQPGGSLRL |
| | | SCAASGFKITHYTMGWFRQA |
| | | PGKEREFVSRITWGGDNTFY |
| | | SNSVKGRFTISRDNSKNTLY |
| | | LQMNSLRAEDTAVYYCAAGS |
| | | TSTATPLRVDYWGQGTLVTV |
| | | SS |
| 17 | H72 | EVQLVESGGGLVQPGGSLRL |
| | | SCAASGFKITHYTTGWFRQA |
| | | PGKEREFVSRITWGGDNTFY |
| | | SNSVKGRFTISRDNSKNTLY |
| | | LQMNSLRAEDTAVYYCAAGS |
| | | TSTATPLRVDYWGQGTLVTV |
| | | SS |
| 18 | Short hinge | KTHTCPPCPAPELLGGPSVF |
| | hIgG1 Fc | LFPPKPKDTLMISRTPEVTC |
| | agly | VVVDVSHEDPEVKFNWYVDG |
| | | VEVHNAKTKPREEQYNSAYR |
| | | VVSVLTVLHQDWLNGKEYKC |
| | | KVSNKALPAPIEKTISKAKG |
| | | QPREPQVYTLPPSRDELTKN |
| | | QVSLTCLVKGFYPSDIAVEW |
| | | ESNGQPENNYKTTPPVLDSD |
| | | GSFFLYSKLTVDKSRWQQGN |
| | | VFSCSVMHEALHNHYTQKSL |
| | | SLSPGC |
| 33 | Long hinge | AEPKSCDKTHTCPPCPAPEL |
| | hIgG1 Fc | LGGPSVFLFPPKPKDTLMIS |
| | agly | RTPEVTCVVVDVSHEDPEVK |
| | | FNWYVDGVEVHNAKTKPREE |
| | | QYNSAYRVVSVLTVLHQDWL |
| | | NGKEYKCKVSNKALPAPIEK |
| | | TISKAKGQPREPQVYTLPPS |
| | | RDELTKNQVSLTCLVKGFYP |
| | | SDIAVEWESNGQPENNYKTT |
| | | PPVLDSDGSFFLYSKLTVDK |
| | | SRWQQGNVFSCSVMHEALHN |
| | | HYTQKSLSLSPGK |

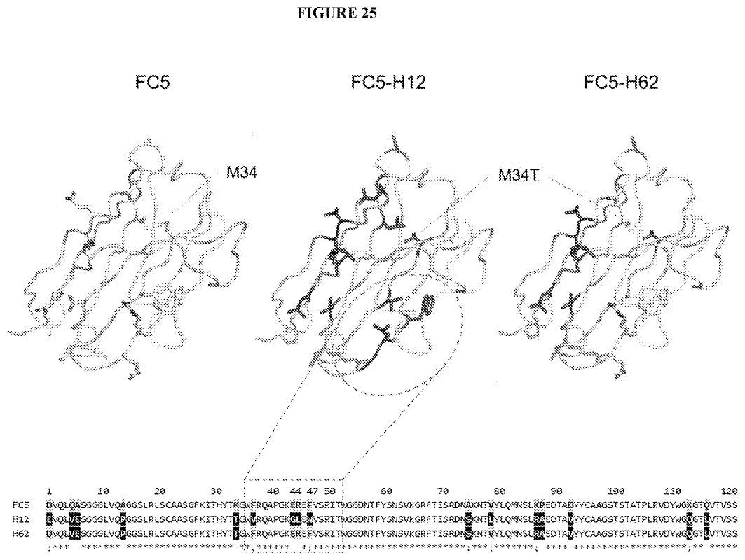

A computer model of FC5 was generated by combining assignments of the backbone atoms from an NMR study of FC5, that revealed structural features of the FC5 framework and CDR-H3, with an analysis of likely conformations for CDRs H1 and H2, using the PyIgClassify web server (http://dunbrack2.fccc.edu/PyIgClassify/). This information was used to guide the selection of X-ray crystal structures from the Protein Data Bank (PDB) as templates. The models combined coordinates for the framework and CDRs HI and H2 from crystal structures and for the CDR-H3 from the NMR study. Models of the humanized variant designs H62 and H12 are shown in FIG. 25. The side-chain residues on the surface of the corresponding human VH/VL interface are indicated.

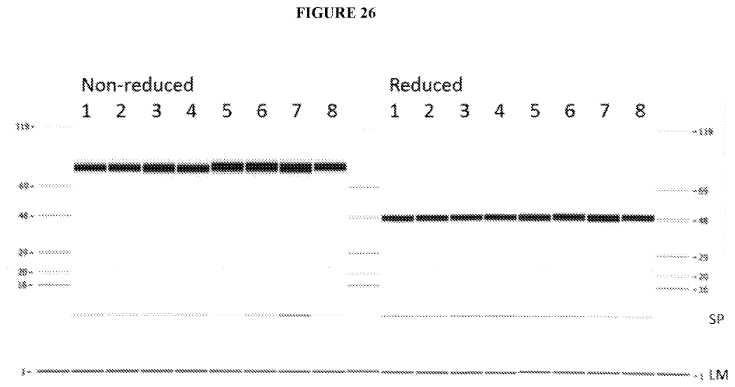

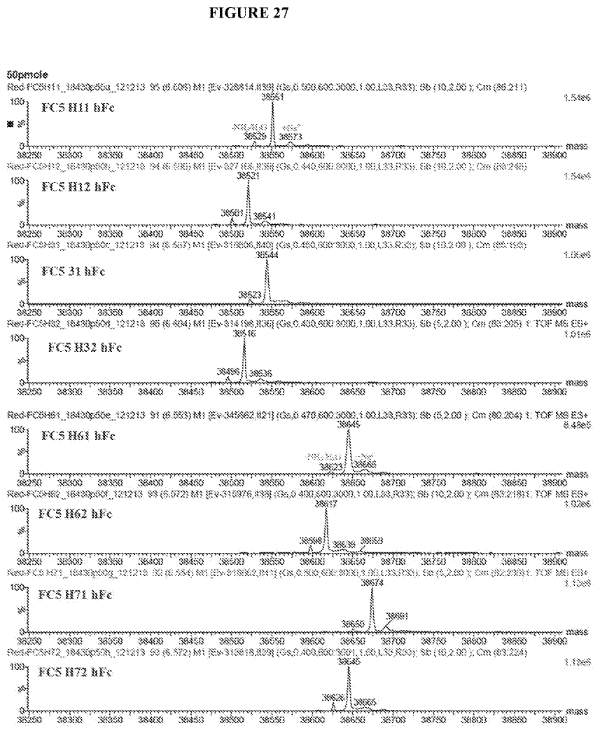

FC5 humanization variants were expressed as hIgG1 Fc fusions (aglycosylated), with the short hinge linker (KTHTCPPCP (SEQ ID NO:19)), in DG44 CHO cells and purified by Protein A Sepharose affinity chromatography. For preparation of the conditioned medium, transfected CHO cells from an unsorted pool were expanded in serum-free media, grown to high density, fed with supplements, and shifted to a reduced temperature. Cultures were held at this reduced temperature for 14 days and then harvested by centrifugation and clarified by 0.45-micron filtration. 1.8 L of clarified and filtered culture medium was loaded onto a 25 mL Protein A column (MabSelect SuRe; GE). The column was washed with 20 mM Na₂HPO₄ pH 7.4, 150 mM NaCl and FC5-hFc molecules were eluted with 25 mM NaH₂PO₄ pH 2.8, 100 mM NaCl, neutralized with 12.5 mM Na₂HPO₄ pH 8.6 diluted from a 0.5 M stock solution. Purified proteins were analyzed for size and homogeneity by denaturing microfluidic capillary electrophoresis (LabChip GXII Protein Express Assay; Perkin Elmer). All molecules showed the expected size of a VHH-hFc dimer under non-reducing conditions and of the corresponding monomer under reducing conditions (FIG. 26), with no apparent aggregates or proteolysis products. Intact mass spectrometry analysis of reduced samples confirmed correspondence between predicted and measured mass for all FC5-hFc humanization variants (within 5 Da; FIG. 27).

Size exclusion chromatography analysis was performed for all FC5-hFc variants on a Biosep s3000 7.8×300 mm silica column (Phenomenex) at 0.6 mL/min in 100 mM Na₂HPO₄, 200 mM NaCl, pH 6.8. All humanization variants eluted as single peaks (FIG. 28), but H11, H12, H31, H32, and H72 had significantly delayed elution times, indicative of interactions with the silica resin. Notably H31 and H32 showed extensive peak broadening with shallow ascending and descending shoulders.

Additional tests of molecular stability were performed on FC5-hFc humanization variants. Thermal stability profiles generated by differential scanning calorimetry (VP-DSC, MicroCal) for all variants were similar, with melting temperatures for the VHH and CH2 domains in the range of 55−62° C. and the melting temperature of the CH3 domain >90° C. (Table 5). Two main melting transitions observed by DSC at temperatures Tm1, characterizing unfolding of the hFc CH2 domain and the FC5 VHH, and Tm2, characterizing the unfolding of the hFc CH3 domain.

TABLE 5
Thermal Stability of FC5-hFc Humanization Variants

| | Tm₁ | Tm₂ |
|---|---|---|
| Molecule | CH2/VHH | CH3 |
| FC5 Fc hIgG1 | 56.5 | 84.4 |
| FC5 H11 hFc | 59.9 | 92.6 |
| FC5 H12 hFc | 55.6 | 91.6 |
| FC5 H31 hFc | 59.5 | 92.3 |
| FC5 H32 hFc | 56.0 | 91.6 |
| FC5 H61 hFc | 61.5 | 92.2 |
| FC5 H62 hFc | 56.8 | 91.9 |
| FC5 H71 hFc | 61.3 | 92.2 |
| FC5 H72 hFc | 56.7 | 92.1 |

Small amounts of fine precipitate were visible in the samples of H11, H12, and H32 variants after four months of storage at 4° C.

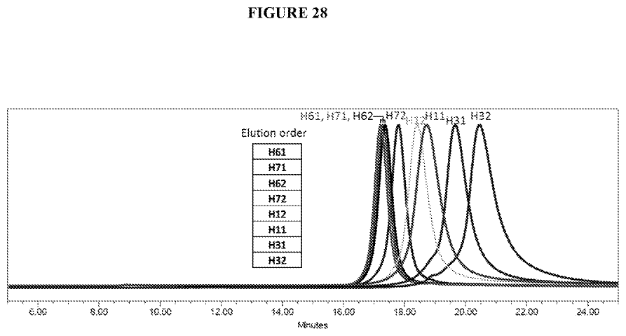

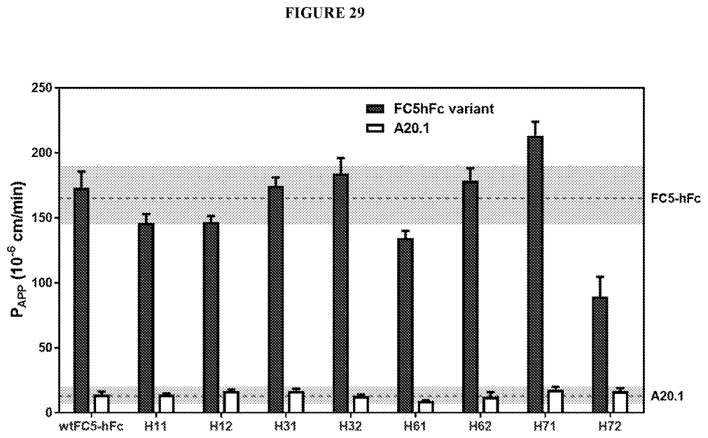

The biological activity of the FC5-hFc humanization variants was assessed in the in vitro BBB transwell cell culture assay using SV-ARBEC cells as described in Example 2. The molecules were co-applied to the upper chamber of the in vitro BBB model at 1.25 μM in paired combinations (A20.1 control antibody and test molecule) and quantified by MRM in the bottom compartment. An apparent permeability (P_{APP}) value for each sample was calculated over 60 min. Each test molecule was tested in triplicate transwell cultures. Results from this analysis for FC5-hFc and the humanization variants are shown in FIG. 29. All the test samples, except the H72 variant, showed FC5 mediated transport with average P_{APP} values of 130-220×10⁻⁶ cm/min, and most fell within the historical range for wild type FC5-hFc (145-190×10⁻⁶ cm/min). H72 showed significantly lower transport activity than the other humanization variants

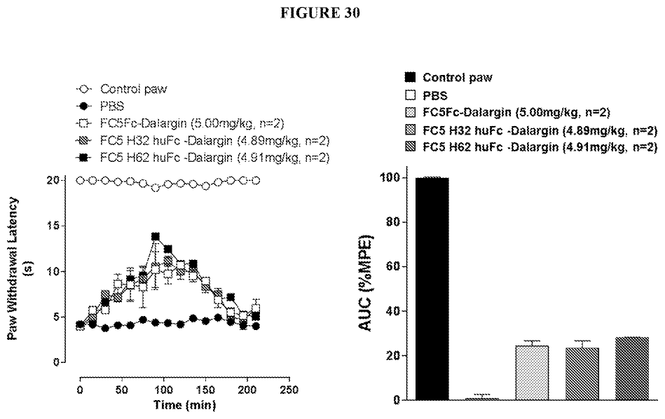

To test if the FC5-hFc humanization variants showed BBB transport activity in vivo, wild-type FC5-hFc as well as the H32 and H62 variants were tested in the Hargreaves model of inflammatory hyperalgesia. In this model the constructs were chemically conjugated to dalargin-cysteine, administered by i.v. injection, and pain response measured overtime (as described in Example 1). As shown in FIG. 30, systemic dosing of the FC5-hFc dalargin conjugates induced a significant analgesic response that is not significantly different between wild type and either the H32 or H62 humanization variants. The integrated analgesic response (AUC values) for the three molecules were similar, ranging from 20-30% of the maximum possible effect.

### Example 5: Engineering a Second Disulfide in FC5 to Protect Against the Degradation in Rat Serum

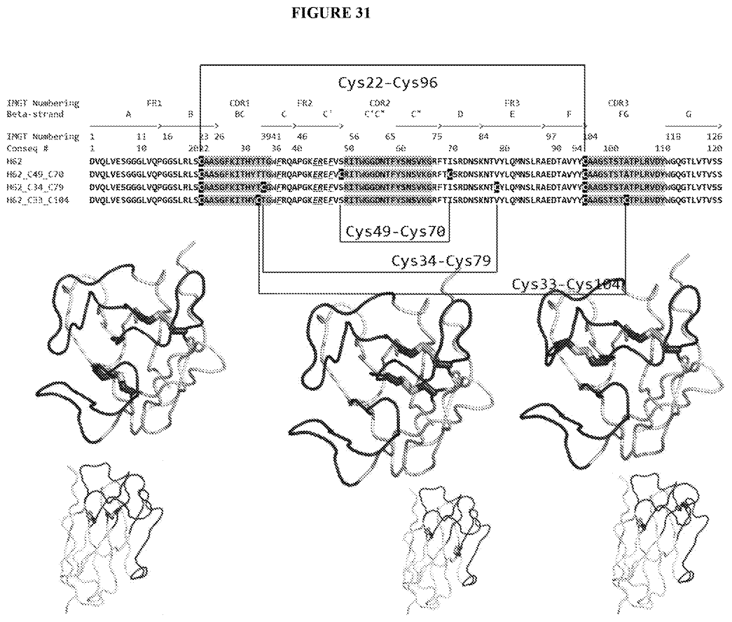

A series of nine constructs (Table 6) were designed to stabilize FC5 from degradation by insertion of a second disulfide at the following sites: S49C-170C, T34C-V79C, and T33C paired with S102C, T103C, A104C or T105C in FC5 humanization design H62, as well as S49C-170C, T34C-V79C, and T33C-T103C in FC5 humanization design H32. Molecular models of the engineered disulfides within the FC5 VHH are shown in FIG. 31. In the models, the engineered cysteines are oriented at positions that would allow for the disulfides to form. The T34C-V79C creates a disulfide connecting framework region 2 (between CDR1 and CDR2) to framework region 3 (between CDR2 and CDR3), S49C-170C creates a different disulfide connecting framework regions 2 and 3, and the S33C series create disulfides connecting CDRs 1 and 3.

TABLE 6
Exemplary Disulfide Stabilized FCS Sequences

| SEQ | | | |
|---|---|---|---|
| ID | | Position | |
| NO. | 4D# | modified | Amino Acid Sequence |
| 20 | 4662 | H62- | DVQLVESGGGLVQPGGSLRL |
| | | S49C/I70C | SCAASGFKITHYTTGWFRQA |
| | | | PGKEREFVCRITWGGDNTFY |
| | | | SNSVKGRFTCSRDNSKNTVY |
| | | | LQMNSLRAEDTAVYYCAAGS |
| | | | TSTATPLRVDYWGQGTLVTV |
| | | | SS |
| 21 | 4663 | H32- | EVQLVESGGGLVQPGGSLRL |
| | | S49C/I70C | SCAASGFKITHYTTGWFRQA |
| | | | PGKGLEFVCRITWGGDNTFY |
| | | | SNSVKGRFTCSRDNSKNTVY |
| | | | LQMNSLRAEDTAVYYCAAGS |
| | | | TSTATPLRVDYWGQGTLVTV |
| | | | SS |
| 22 | 4664 | H32- | EVQLVESGGGLVQPGGSLRL |
| | | T34C/V79C | SCAASGFKITHYTCGWFRQA |
| | | | PGKGLEFVSRITWGGDNTFY |
| | | | SNSVKGRFTISRDNSKNTCY |
| | | | LQMNSLRAEDTAVYYCAAGS |
| | | | TSTATPLRVDYWGQGTLVTV |
| | | | SS |
| 23 | 4665 | H62- | DVQLVESGGGLVQPGGSLRL |
| | | T34C/V79C | SCAASGFKITHYTCGWFRQA |
| | | | PGKEREFVSRITWGGDNTFY |
| | | | SNSVKGRFTISRDNSKNTCY |
| | | | LQMNSLRAEDTAVYYCAAGS |
| | | | TSTATPLRVDYWGQGTLVTV |
| | | | SS |
| 24 | 4666 | H62- | DVQLVESGGGLVQPGGSLRL |
| | | T33C/S102C | SCAASGFKITHYCTGWFRQA |
| | | | PGKEREFVSRITWGGDNTFY |
| | | | SNSVKGRFTISRDNSKNTVY |
| | | | LQMNSLRAEDTAVYYCAAGS |
| | | | TCTATPLRVDYWGQGTLVTV |
| | | | SS |
| 25 | 4667 | H62- | DVQLVESGGGLVQPGGSLRL |
| | | T33C/T103C | SCAASGFKITHYCTGWFRQA |
| | | | PGKEREFVSRITWGGDNTFY |
| | | | SNSVKGRFTISRDNSKNTVY |
| | | | LQMNSLRAEDTAVYYCAAGS |
| | | | TSCATPLRVDYWGQGTLVTV |
| | | | SS |
| 26 | 4668 | H62- | DVQLVESGGGLVQPGGSLRL |
| | | T33C/A104C | SCAASGFKITHYCTGWFRQA |
| | | | PGKEREFVSRITWGGDNTFY |
| | | | SNSVKGRFTISRDNSKNTVY |
| | | | LQMNSLRAEDTAVYYCAAGS |
| | | | TSTCTPLRVDYWGQGTLVTV |
| | | | SS |
| 27 | 4669 | H62- | DVQLVESGGGLVQPGGSLRL |
| | | T33C/T105C | SCAASGFKITHYCTGWFRQA |
| | | | PGKEREFVSRITWGGDNTFY |
| | | | SNSVKGRFTISRDNSKNTVY |
| | | | LQMNSLRAEDTAVYYCAAGS |
| | | | TSTACPLRVDYWGQGTLVTV |
| | | | SS |
| 28 | 4670 | H32- | EVQLVESGGGLVQPGGSLRL |
| | | T33C/T103C | SCAASGFKITHYCTGWFRQA |
| | | | PGKGLEFVCRITWGGDNTFY |
| | | | SNSVKGRFTCSRDNSKNTVY |
| | | | LQMNSLRAEDTAVYYCAAGS |
| | | | TSCATPLRVDYWGQGTLVTV |
| | | | SS |

The constructs were expressed in CHO cells and purified by Protein A Sepharose chromatography. Table 7 shows a list of the nine constructs and the purification yields from 250 mL of conditioned medium.

TABLE 7
List of Constructs

| | 4D# | Plasmid1 | Plasmid 2 | Protein | Yield mg | Conc. mg/ml |
|---|---|---|---|---|---|---|
| 1 | 4662 | YL1289 | GC058 | FC5-H62(S49C/170C)-G4S-Li81 T299A hulgG1, kappa | 3 | 1.34 |
| 2 | 4663 | YL1290 | GC058 | FC5-H32(S49C/170C)-G4S-Li81 T299A hulgG1, kappa | 0.5 | 0.81 |
| 3 | 4664 | YL1291 | GC058 | FC5-H32(T34C/V79C)-G4S-Li81 T299A hulgG1, kappa | 2 | 0.96 |
| 4 | 4665 | YL1292 | GC058 | FC5-H62(T34C/V79C)-G4S-Li81 T299A hulgG1, kappa | 6 | 1.49 |
| 5 | 4666 | YL1293 | GC058 | FC5-H62(T33C/S102C)-G4S-Li81 T299A hulgG1, kappa | 3 | 0.64 |
| 6 | 4667 | YL1294 | GC058 | FC5-H62(T33C/T103C)-G4S-Li81 T299A hulgG1, kappa | 3.7 | 0.75 |
| 7 | 4668 | YL1295 | GC058 | FC5-H62(T33C/A104C)-G4S-Li81 T299A hulgG1, kappa | 2.4 | 0.86 |
| 8 | 4669 | YL1296 | GC058 | FC5-H62(T33C/T105C)-G4S-Li81 T299A hulgG1, kappa | 1 | 1.83 |
| 9 | 4670 | Yl1297 | GC058 | FC5-H32(T33C/T103C)-G4S-Li81 T299A hulgG1, kappa | 1 | 0.8 |

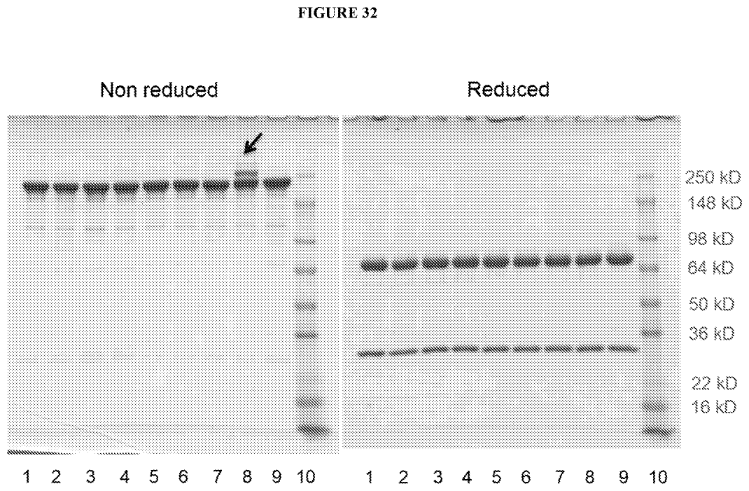

Expression levels of the constructs were estimated at 7-20 mg/L by Octet. For purification, clarified and filtered culture media were loaded onto 0.5-0.8 mL protein A columns, washed with 5 column volumes of 20 mM Na₂HPO₄ pH 7.4, 100 mM NaCl, and then with 25 mM NaH₂PO₄ pH 5.5, 100 mM NaCl. FC5-Li81 was eluted from the columns with 25 mM NaH₂PO₄ pH 2.8, 100 mM NaCl pH 2.8 and neutralized with 25 mM Na₂HPO₄ pH 8.6 diluted from a 0.5 M stock solution. The protein content of the eluted samples was estimated from the absorbance at 280 nm using an extinction coefficient of 1.4 for a 1 mg/mL solution. SDS-PAGE analysis shown in FIG. 32 confirmed expression of the HC and LC and assembly into the characteristic HC2LC2 tetrameric complex under non-reducing conditions. Sample 4669 showed some scrambling of disulfides reflected in the heterogeneity in bands seen under non-reducing conditions (denoted with arrow). The FC5-Li81 samples all showed the expected size exclusion chromatography profiles, each eluting as single prominent peaks with molecular masses of ˜165 kDa.

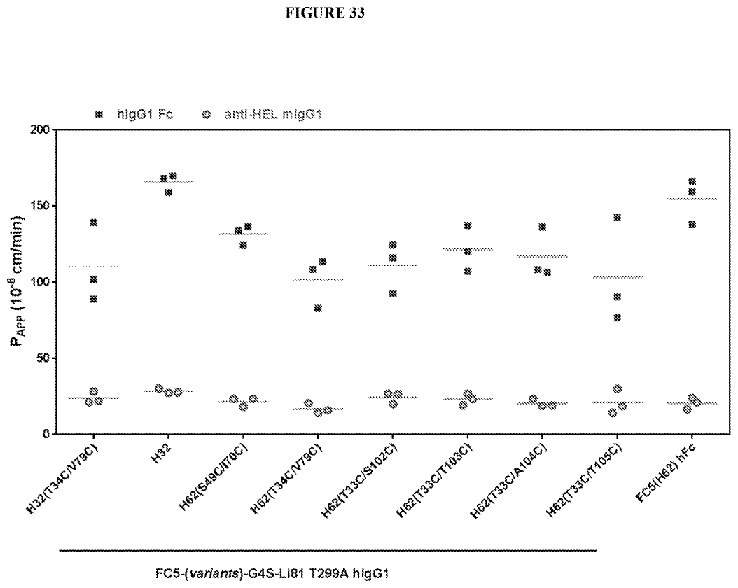

The biological activity of the disulfide stabilized FC5-(G₄S)1-Li81 samples were assessed in the in vitro BBB transwell cell culture assay on SV-ARBEC cells as described in Example 2. The molecules were co-applied to the upper chamber of the in vitro BBB model in paired combinations (Anti-HEL control antibody and test molecule) and quantified by MRM in the bottom compartment; A P_{APP} value for each sample was calculated over 90 min. The (top chamber) input concentration was between 1.5 and 3 μM (linear phase) with equimolar input of the co-administered control antibody. Results from this analysis for 7 of the disulfide stabilized constructs are shown in FIG. 33. All of the test samples showed FC5 mediated transport with average P_{APP} values of 100-130×10′ cm/min. Transport activity of FC5-(G₄S)1-Li81 was not significantly altered by insertion of the second disulfide.

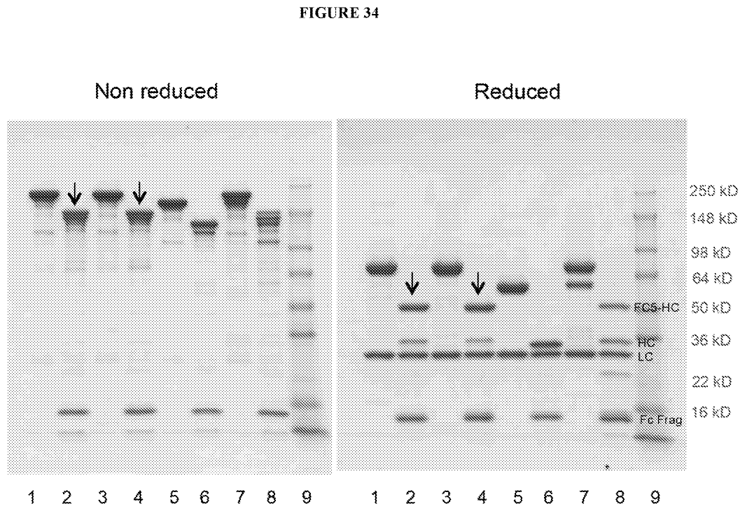

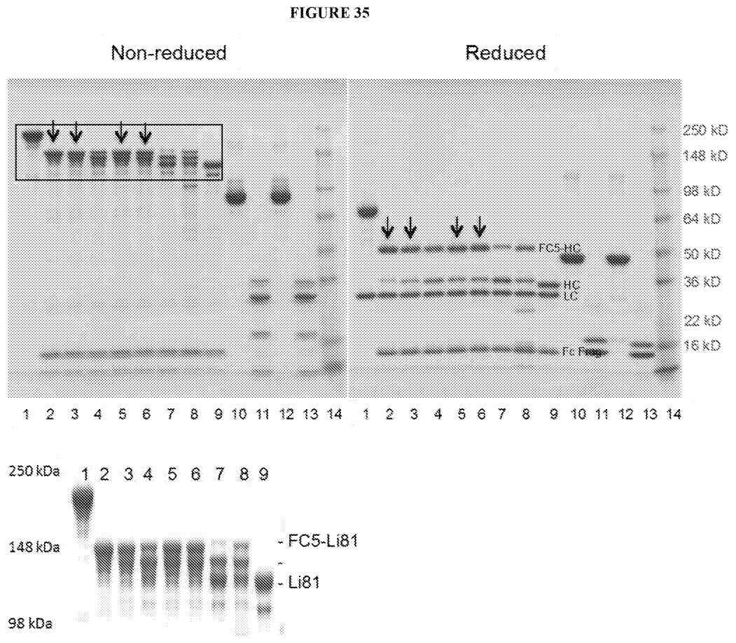

To evaluate if the inserted cysteines/disulfides stabilized the constructs against proteolysis, samples were incubated with pepsin at a 1:30 (pepsin:sample) ratio for 2.5 h at 37° C. and analyzed by SDS-PAGE. FIGS. 34 and 35 show results from this analysis. Pepsin digestion of Li81 (FIG. 34 lane 6) led to formation of the characteristic Fab′2 fragment (100 kDa) and low molecular weight Fc fragments that are routinely seen when an antibody is treated with pepsin and specifically described for Li81 mAb (Pepinsky et al., J Pharmacol Exp Ther, 339:519-529 (2011)). Pepsin digestion of FC5-H32-G₄S-Li81 (FIG. 34 lane 8) led to a series of slightly larger fragments consistent with the presence of the FC5 VIM and fragmentation within the FC5 domain. As seen with Li81 mAb, pepsin treatment of this construct also led to cleavage of the Fc into low molecular weight fragments. In contrast, FC5-H32(T34C/V79C)-G₄S-Li81 and FC5-H32(T33C/T103C)-G₄S-Li81 showed less cleavage of the bands specific to the FC5 domain, indicating stabilization of the FC5. Pepsin digestion of FC5-H62-G₄S-Li81 constructs (FIG. 35) also led to changes in the fragmentation patterns of bands of similar molecule weights. Notably FC5-H62(S49C/I70C)-G₄S-Li81, FC5-H62(T34C/V79C)-G₄S-Li81, FC5-H62(T33C/T103C)-G₄S-Li81, and FC5-H62(T33C/A104C)-G₄S-Li81 were more stable than FC5-H62(T33C/S102C)-G₄S-Li81 and FC5-H62(T33C/T105C)-G₄S-Li81. These findings indicate that the addition of a second disulfide can protect the FC5 VHH from pepsin treatment and that the position of the added disulfide can impact the extent to which the FC5 is stabilized. As an independent measure of stabilization, the samples were also analyzed for changes in thermal stability by differential scanning calorimetry. All of the disulfide engineered constructs showed a 12-13° increase in Tm of the transition corresponding to FC5 with a value of 60° C. without stabilization and 72.7-73.2° C. for all of constructs with the engineered disulfides.

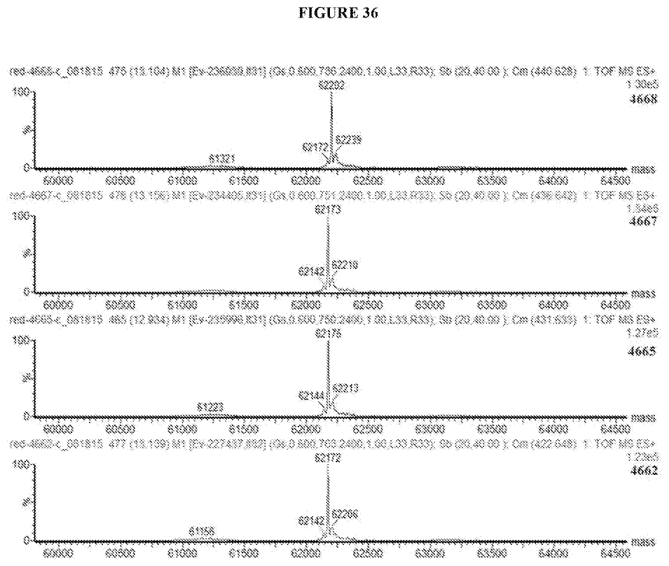

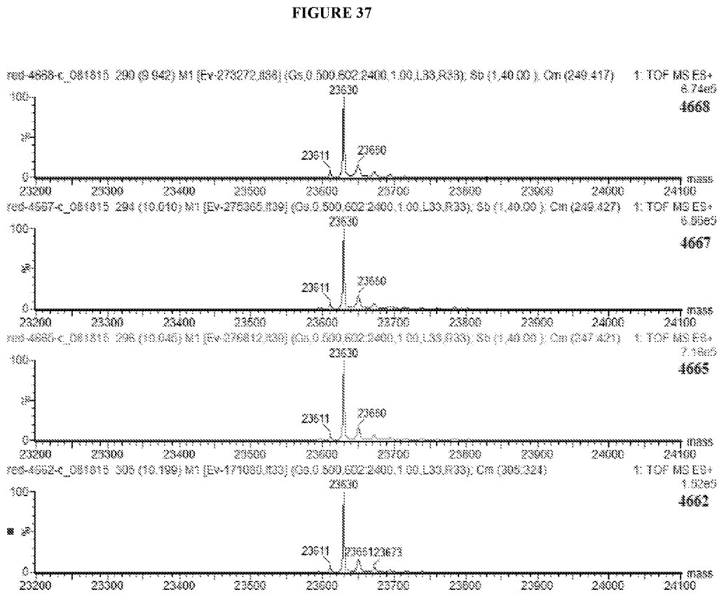

Samples 4662, 4665, 4667, and 4668 were subjected to extensive characterization by mass spectrometry and stability assessment in rat serum. First, intact mass analysis of the reduced heavy chain 4662 (predicted mass 62,172.0 Da, observed mass 62172 Da), 4665 (predicted mass 62,172.0 Da, observed mass 62175 Da), 4667 (predicted mass 62,170.0 Da, observed mass 62173 Da), and 4668 (predicted mass 62,200.0 Da observed mass 62202 Da) samples and light chains (predicted mass 23,628.4 Da, observed mass 23630 Da) showed that the major components in all samples were the predicted light chain and heavy chain. The corresponding deconvoluted spectra are shown in FIGS. 36 and 37. No unexpected modifications on the major component were observed.

Serum stability assessment was performed by incubating 4662, 4665, 4667, and 4668 samples for 70 h at 37° C. in rat serum, then purifying them on Ig select and subjecting them to peptide mapping under reducing and non-reducing conditions as described in Example 3. Samples were spiked into Wistar rat serum at 100 μg/mL and purified on Ig-Select at 504 resin/mL of serum. Each sample was compared to its control without serum treatment. Analysis of disulfide peptide maps of the control and 70h samples of FC5-H62-G₄S-Li81 4662, 4665, 4667, and 4668 constructs showed that the major disulfide linkages are all as expected for each construct (Tables 8 to 11).

TABLE 8
List of major disulfide linked peptides in FC5-H62(S49C/I70C)- G₄S-Li81 (4662)

| | | Calculated | | | | | | | | |
|---|---|---|---|---|---|---|---|---|---|---|
| | | Peptide | RT | | Charge | Mass | Intensity (Counts)* | | Recovery (%) | |
| Peptide | Cys No. | Mass (Da) | (Min) | m/z | State | (Da) | control | 70 h | control | 70 h |
| LC19-24═LC62-91 | C1═C2 | 3951.793 | 54.5 | 1318.262 | 3 | 3951.763 | 1423566 | 1489651 | 100 | 105 |
| LC128-143═LC192-208 | C3═C4 | 3555.749 | 49.6 | 1186.247 | 3 | 3555.718 | 2594301 | 2428337 | 100 | 94 |
| LC209-215═HC347-350 | C5═C9 | 1260.486 | 22.9 | 631.245 | 2 | 1260.475 | 137555 | 134140 | 100 | 97 |
| LC209-215═HC347-350 | C5═C9 | 1292.458 | 23.9 | 647.232 | 2 | 1292.448 | 5111 | 4429 | | |
| | (trisulfide) | | | | | | | | | |
| HC20-28═HC88-108 | C1═C4 | 3027.395 | 35.6 | 1010.132 | 3 | 3027.374 | 113731 | 100069 | 100 | 88 |
| HC44-50═HC68-72 | C2═C3 | 1547.697 | 29.3 | 774.851 | 2 | 1547.685 | 225228 | 190093 | 100 | 84 |
| HC147-162═HC215-249 | C5═C6 | 5382.348 | 52.7 | 1346.586 | 4 | 5382.312 | 144594 | 191955 | 100 | 130 |
| HC147-162═HC215-249 | | 5398.343 | 50.5 | 1350.584 | 4 | 5398.306 | 17557 | 17286 | | |
| oxM161 | | | | | | | | | | |
| HC141-162═HC215-249 | | 5414.338 | 48.1 | 1811.445 | 3 | 5431.311 | 3411 | 6701 | | |
| oxM161&W235 | | | | | | | | | | |
| HC262-275═HC276-338 | C7═C8 | 7916.919 | 58.5 | 1584.381 | 5 | 7916.863 | 1796647 | 1925570 | 100 | 106 |
| HC262-275═HC276-338 | | 7948.909 | 57.7 | 1590.778 | 5 | 7948.852 | 112057 | 105877 | | |
| oxW286 | | | | | | | | | | |
| HC351-376═HC351-376 | C10═C10, | 5454.783 | 57.5 | 1364.695 | 4 | 5454.748 | 1196705 | 1351732 | 100 | 113 |
| | C11═C11 | | | | | | | | | |
| HC384-402═HC449-450 | C12═C13 | 2328.098 | 34.9 | 777.034 | 3 | 2328.078 | 1643485 | 1860372 | 100 | 113 |
| HC489-498═HC545-567 | C14═C15 | 3844.824 | 40.8 | 1282.606 | 3 | 3844.794 | 1124492 | 1207385 | 100 | 104 |
| HC489-498═HC545-567 | | 3860.818 | 38.6 | 1287.945 | 3 | 3860.810 | 141956 | 117190 | | |
| oxM.556 | | | | | | | | | | |
| HC489-498═HC545-567 | | 3876.813 | 40.2 | 1293.266 | 3 | 3876.774 | 15506 | 15738 | | |
| oxW546 | | | | | | | | | | |
| HC489-498═HC.545-567 | | 3892.808 | 37.9 | 1298.603 | 3 | 3892.785 | 50953 | 39773 | | |
| oxM.556&oxW546 | | | | | | | | | | |

*Intensities normalized to 2 heavy chain Fc region and 2 light chain peptides in the control sample.

TABLE 9
List of major disulfide linked peptides in FC5-H62(T34C/V79C)-G₄S-Li81 (4665)

| | | Calculated | | | | | | | | |
|---|---|---|---|---|---|---|---|---|---|---|
| | | Peptide | RT | | Charge | | Intensity (Counts)* | | Recovery (%) | |
| Peptide | Cys No. | Mass (Da) | (Min) | m/z | State | Mass (Da) | control | 70 h | control | 70 h |
| LC19-24═LC62-91 | C1═C2 | 3951.793 | 54.6 | 1318.260 | 3 | 3951.757 | 1425942 | 1368097 | 100 | 96 |
| LC128-143═LC192-208 | C3═C4 | 3555.749 | 49.8 | 1186.245 | 3 | 3555.712 | 2311821 | 2244808 | 100 | 97 |
| LC209-215═HC347-350 | C5═C9 | 1260.486 | 22.9 | 631.243 | 2 | 1260.470 | 117616 | 111744 | 100 | 94 |
| LC209-215═HC347-350 | C5═C9 | 1292.458 | 23.9 | 647.229 | 2 | 1292.443 | 4941 | 3708 | | |
| | (trisulfide) | | | | | | | | | |
| HC20-28═HC88-108 | C1═C4 | 3027.395 | 35.7 | 1010.128 | 3 | 3027.361 | 339597 | 238932 | 100 | 70 |
| HC29-38═HC77-87 | C2═C3 | 2622.1931 | 41.8 | 875.062 | 3 | 2622.163 | 223896 | 164272 | 100 | 77 |
| HC29-38═HC77-87 oxM83 | | 2638.1880 | 38.5 | 880.3936 | 3 | 2638.157 | 50152 | 37979 | | |
| HC29-38═HC77-87 oxW36 | | 2654.1829 | 39.7 | 885.7226 | 3 | 2654.141 | 3300 | 4532 | | |
| HC29-38═HC77-87 oxM83 & | | 2638.1880 | 38.5 | 880.3936 | 3 | 2638.157 | 50152 | 37979 | | |
| W36 | | | | | | | | | | |
| HC147-162═HC215-249 | C5═C6 | 5382.348 | 52.8 | 1346.584 | 4 | 5328.304 | 281793 | 218362 | 100 | 75 |
| HC147-162═HC215-249 | | 5398.343 | 50.7 | 1350.582 | 4 | 5398.297 | 34092 | 21025 | | |
| oxM161 | | | | | | | | | | |
| HC147-162═HC215-249 | | 5414.338 | 48.3 | 1811.435 | 3 | 5431.281 | 12215 | 6748 | | |
| oxM161&W235 | | | | | | | | | | |
| HC262-275═HC276-338 | C7═C8 | 7916.919 | 58.6 | 1584.381 | 5 | 7916.863 | 1829138 | 2057157 | 100 | 107 |
| HC262-275═HC276-337 | | 7948.909 | 57.7 | 1590.779 | 5 | 7948.857 | 122091 | 39398 | | |
| oxW286 | | | | | | | | | | |
| HC351-376═HC351-376 | C10═C10, | 5454.783 | 57.6 | 1364.693 | 4 | 5454.740 | 1183538 | 1159419 | 100 | 98 |
| | C11═C11 | | | | | | | | | |
| HC384-402═HC449-450 | C12═C13 | 2328.098 | 35.0 | 777.031 | 3 | 2328.069 | 1608257 | 1690745 | 100 | 105 |
| HC489-498═HC545-567 | C14═15 | 3844.824 | 40.9 | 1282.600 | 3 | 3844.777 | 1207967 | 1093265 | 100 | 90 |
| HC489-498═HC545-567 | | 3860.818 | 38.7 | 1287.935 | 3 | 3860.783 | 129822 | 115462 | | |
| oxM556 | | | | | | | | | | |
| HC489-498═HC545-567 | | 3876.813 | 40.3 | 1293.265 | 3 | 3876.764 | 15835 | 12474 | | |
| oxW546 | | | | | | | | | | |
| HC489-498═HC545-567 | | 3892.508 | 37.9 | 1298.596 | 3 | 3892.765 | 43580 | 38199 | | |
| oxM556 & oxW546 | | | | | | | | | | |

*intensities normalized to 2 heavy chain Fc region and 2 light chain peptides in the control sample.

TABLE 10
List of major disulfide linked peptides in FC5-H62(T33C/T103C)-G₄S-Li81 (4667)

| | | Calculated | | | | | | | | |
|---|---|---|---|---|---|---|---|---|---|---|
| | | Peptide | RT | | Charge | Mass | Intensity (Counts)* | | Recovery (%) | |
| Peptide | Cys No. | Mass (Da) | (Min) | m/z | State | (Da) | control | 70 h | control | 70 h |
| LC19-24═LC63-91 | C1═C2 | 3951.793 | 54.7 | 1318.252 | 3 | 3951.731 | 1503398 | 1477844 | 100 | 98 |
| LC128-143═LC192-208 | C3═C4 | 3555.749 | 49.9 | 1186.235 | 3 | 3555.690 | 2509561 | 2326577 | 100 | 93 |
| LC209-215═HC347-350 | C5═C9 | 1260.486 | 22.9 | 631.243 | 2 | 1260.470 | 126372 | 127719 | 100 | 101 |
| LC209-215═HC347-350 | C5═C9 | 1292.458 | 23.9 | 647.229 | 2 | 1292.443 | 1145 | 1193 | | |
| | (trisulfide) | | | | | | | | | |
| HC20-28═HC29-38═HC88-108 | C1═C3 | 4309.933 | 39.3 | 1078.472 | 4 | 4309.857 | 558117 | 512343 | 100 | 92 |
| HC20-28═HC29-38═BC88-108 | C2═C4 | 4341.923 | 37.8 | 1448.291 | 3 | 4341.849 | 37311 | 37369 | | |
| oxW36 | | | | | | | | | | |
| HC20-28═HC29-38═HC88-108/ | C1═C3 | 4341.905 | 39.8 | 1086.466 | 4 | 4341.830 | 70183 | 60037 | | |
| HC20-28═HC29-38═HC88-108 | C2═C4/ | | | | | | | | | |
| | C1═C3 | | | | | | | | | |
| | C2═C4 | | | | | | | | | |
| | (trisulfide) | | | | | | | | | |
| HC147-162═HC215-249 | C5═C6 | 5382.348 | 52.8 | 1346.575 | 4 | 5382.269 | 191461 | 207595 | 100 | 106 |
| HC147-162═HC215-249 oxM161 | | 5398.343 | 50.7 | 1350.573 | 4 | 5398.261 | 25976 | 22073 | | |
| HC262-275═HC276-338 | C7═C8 | 7916.919 | 58.7 | 1584.366 | 5 | 7916.790 | 2120976 | 3125042 | 100 | 98 |
| HC262-275═HC276-338 oxW286 | | 7948.909 | 57.8 | 1590.766 | 5 | 7948.791 | 163884 | 123893 | | |
| HC351-376═HC351-376 | C10═C10, | 5454.783 | 57.7 | 1364.683 | 4 | 5454.701 | 1253039 | 1295230 | 100 | 103 |
| | C11═C11 | | | | | | | | | |
| HC384-402═HC449-450 | C12═C13 | 2328.098 | 35.0 | 777.026 | 3 | 2328.055 | 1717043 | 1913877 | 100 | 111 |
| HC489-498═HC545-567 | C14═C15 | 3844.824 | 41.0 | 1282.594 | 3 | 3844.757 | 1170518 | 1156914 | 100 | 97 |
| HC459-498═HC545-367 oxM556 | | 3860.518 | 38.7 | 1287.931 | 3 | 3860.770 | 142015 | 127815 | | |
| HC459-498═HC545-567 oxM546 | | 3876.813 | 40.4 | 1293.253 | 3 | 3876.737 | 16963 | 14321 | | |
| HC489-498═HC545-567 oxM556 & | | 3892.808 | 38.0 | 1298.590 | 3 | 3892.745 | 57799 | 44735 | | |
| oxW546 | | | | | | | | | | |

*intensities normalized to 2 heavy chain Fc region and 2 light chain peptides in the control sample.

TABLE 11
List of major disulfide linked peptides in FC5-H62(T33C/A104C)-G₄S-Li81 (4668)

| | | Calculated | | | | | | | | |
|---|---|---|---|---|---|---|---|---|---|---|
| | | Peptide | RT | | Charge | Mass | Intensity (Counts)* | | Recovery (%) | |
| Peptide | Cys No. | Mass (Da) | (Min) | m/z | State | (Da) | control | 70 h | control | 70 h |
| LC19-24═LC62-91 | C1═C2 | 3951.793 | 66.9 | 1318.280 | 3 | 3951.817 | 2250228 | 2381326 | 100 | 106 |
| LC128-143═LC192-208 | C3═C4 | 3555.749 | 58.2 | 1186.263 | 3 | 3555.6765 | 3066559 | 3344356 | 100 | 109 |
| LC209-215═HC347-350 | C5═C9 | 1260.486 | 22.9 | 631.252 | 2 | 1260.487 | 353717 | 380399 | 100 | 107 |
| LC209-215═HC347-350 | C5═C9 | 1292.458 | 23.8 | 647.237 | 2 | 1292.459 | 5879 | 5760 | | |
| | (trisulfide) | | | | | | | | | |
| HC20-28═HC29-38═HC88-108 | C1═C3 | 4339.943 | 39.3 | 1085.996 | 4 | 4339.952 | 244385 | 290784 | 100 | 126 |
| | C2═C4 | | | | | | | | | |
| HC20-28═HC29-38═HC88-108 | | 4371.933 | 37.8 | 1093.995 | 3 | 4371.948 | 7310 | 28718 | | |
| oxW36 | | | | | | | | | | |
| HC20-28═HC29-38═HC88-108/ | C1═C3 | 4371.916 | 39.1 | 1093.990 | 4 | 4371.929 | 14748 | 15359 | | |
| HC20-28═HC29-38═HC88-108 | C2═C4/ | | | | | | | | | |
| | C1═C3 | | | | | | | | | |
| | C2═C4 | | | | | | | | | |
| | (trisulfide) | | | | | | | | | |
| HC147-162═HC215-249 | C5═C6 | 5382.348 | 65.1 | 1346.603 | 4 | 5382.381 | 174158 | 244362 | 100 | 144 |
| HC147-162═HC215-249 oxM161 | | 5398.343 | 62.3 | 1350.600 | 4 | 5398.369 | 2158 | 9770 | | |
| HC262-275═HC276-338 | C7═C8 | 7916.919 | 70.5 | 1584.403 | 5 | 7916.978 | 2414802 | 2772867 | 100 | 121 |
| HC262-275═HC276-338 oxW286 | | 7948.909 | 69.6 | 1590.803 | 5 | 7948.974 | 58684 | 209217 | | |
| HC351-376═HC351-376 | C10═C10, | 5454.783 | 69.4 | 1364.713 | 4 | 5454.821 | 2060734 | 2252746 | 100 | 109 |
| | C11═C11 | | | | | | | | | |
| HC384-402═HC449-450 | C12═C13 | 2328.098 | 34.7 | 777.042 | 3 | 2328.103 | 2123906 | 2092584 | 100 | 99 |
| HC489-498═HC545-567 | C14═C15 | 3844.824 | 40.2 | 1282.619 | 3 | 3844.834 | 1882199 | 1833684 | 100 | 100 |
| HG489-498═HC545-567 oxM556 | | 3860.818 | 38.0 | 773.174 | 5 | 3860.829 | 8516 | 18830 | | |
| HC489-498═HC545-567 oxW546 | | 3876.813 | 39.6 | 1293.283 | 3 | 3876.826 | 7189 | 25999 | | |
| HC489-498═HC545-567 oxM556 & | | 3892.808 | 37.3 | 1298.614 | 3 | 3892.819 | 9536 | 36006 | | |
| oxW546 | | | | | | | | | | |

*Intensities normalized to 2 heavy chain Fc region and 2 light chain peptides in the control sample.

In addition, no mislinkages were detected for the Cys residues of the heavy chains FC5 region and variable region. Analysis showed that the constructs 4667 and 4668 contain a trisulfide bond in the FC5 region, i.e., a trisulfide bond in disulfide-linked peptide cluster containing C1, C2, C3, and C4 [HC 20-28 (C1), HC 88-108 (C3, C4), HC 29-38 (C2)]. The level of the trisulfide was ˜11% in 4667 construct and ˜6% in 4668 construct. Trisulfide linkage between the heavy chain (Cys348) and light chain (Cys215) was the highest (13-16%) in 4807 and 4809 constructs (4807=FC5-H32(E1Q)-G₄S-Li81; 4809=FC5-H62(D1Q)-G₄S-Li81), but low (1-4%) in the remaining constructs 4662, 4665, 4667 and 4668. Only small decreases in the trisulfides levels were observed in the 70 h samples incubated in Wistar rat serum, when compared to the level in controls. No other Cys modifications were observed at a significant level for any of the samples. The reduced Lys-C/tryptic maps of 4-vinylpyridine-treated, non-reduced control and 70 h six pair constructs were also examined to evaluate if there are any significant difference in the level of free thiols in the native samples. Analysis revealed small amounts of free thiols in all samples, and no significant differences between control and 70 h samples. The levels of trisulfides in samples vary by differences in culture conditions and therefore the levels reported are not ment to reflect product-specific attributes.

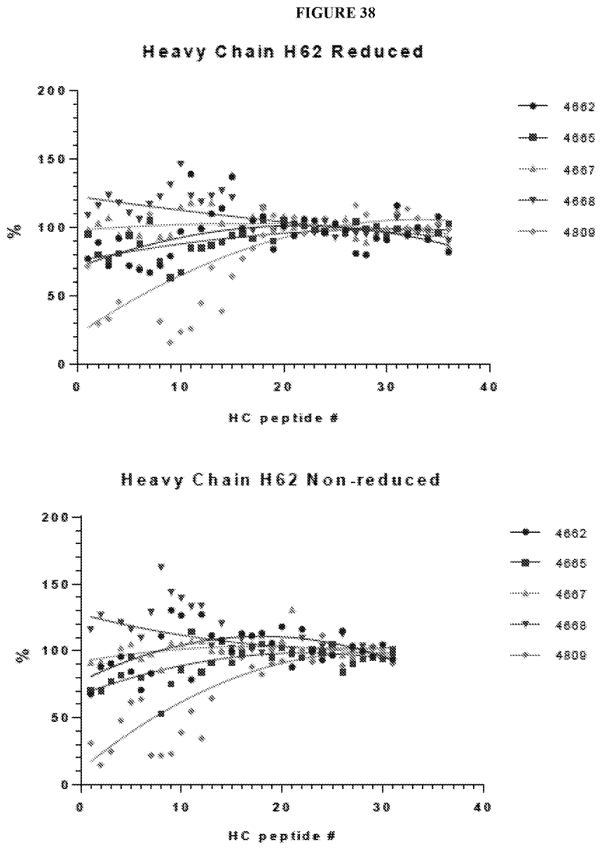

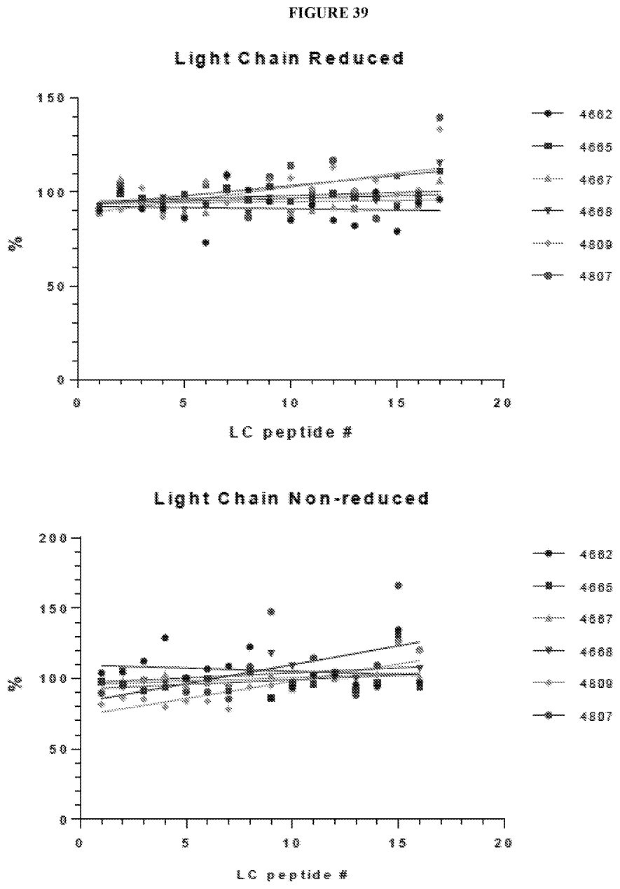

Systematic comparison of the peptide recoveries in the reduced peptide maps of the 70 h samples with those in the maps of the corresponding control samples indicated that previously observed N-terminal clipping of the heavy chain (see Example 3) was significantly reduced/eliminated by addition of a disulfide bridge in the FC5 region of the heavy chain. Results from these analysis are shown in FIGS. 38 and 39. Peptide recovery of the 70 h samples of FC5-H62 mutant constructs 4667 (T33C=T103C) and 4668 (T33C=A104C), with disulfide bonds between C1 (the first Cys) and C3 (the third Cys) and between C2 (the second Cys) and C4 (the fourth Cys), was similar to that for control samples, while peptide recovery of the 70 h samples of FC5-H62 mutant constructs 4662 (S49C=I70C) and 4665 (T34C=V79C), with disulfide bonds between C1 and C4 and between C2 and C3, was about 85% and 70% of that of the control, respectively. These findings indicate that addition of a second disulfide in FC5 protects the FC5 portion of the fusion protein from proteolytic degradation in serum that was seen in FIG. 23 with the wildtype FC5 protein.

The first amino acid in H62 FC5 is an aspartic acid residue. Because antibodies often start with pyroglutamate and this modification could provide protection from amino peptidase activity, we designed a construct (4809) that contained the wildtype H62 FC5 sequence but started with glutamine (D1Q), which would lead to formation of an N-terminal pyroglutamate. Interestingly, when this sample was tested for serum stability (FIG. 38) it was greatly destabilized showing only 30% recovery of the N-terminal peptide versus control. Light chain peptide recovery of 70 h sample was very similar to that of corresponding control sample. The plot for the recovery of the light chain peptides is shown in FIG. 39.

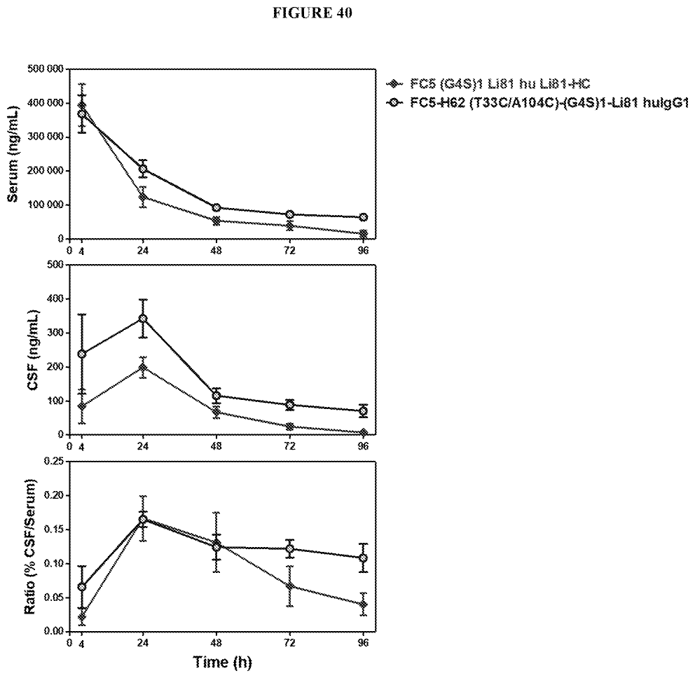

Based on the improvements in the serum stability of H62-T33C/A104C (4D #4668) seen in FIG. 38, it was subjected to PK analysis in rats measuring serum and CSF levels following a 10 mg/kg (˜65 nmol/kg) i.v. administration. Results from this analysis are shown in FIG. 40. Serum and CSF levels for the disulfide stabilized product were higher than the non-stabilized version at all time points. The CSF/serum shown in the bottom panel of FIG. 40 revealed that the disulfide engineering prevented the loss in transport activity over time that was observed with the wild type construct. Brain levels were also measured at 24 h by MRM, where values of FC5, Li81, and hFc reflect concentrations of signature peptides from the respective domains (Table 12).

TABLE 12
Analysis of Brain Levels of FC5-Li81 Samples at 24 h

| | | | Brain IgG peptides | | | |
|---|---|---|---|---|---|---|
| | | Time | (ng/g of tissue) | | | |
| Antibody | Rat | (h) | FC5 | Li81 | hFc | Average |
| FC5-(G4S)1-Li81 hIgG1 | DE7 | 24 | 351 | 401 | 414 | 389 |
| FC5-(G4S)1-Li81 hIgG1 | DE8 | 24 | 442 | 409 | 462 | 438 |
| FC5-H62(T33C/A104C)- | DE9 | 24 | 508 | 657 | 615 | 594 |
| (G4S)1-Li81 hIgG1 | | | | | | |
| FC5-H62(T33C/A104C)- | DE10 | 24 | 639 | 676 | 560 | 625 |
| (G4S)1-Li81 hIgG1 | | | | | | |

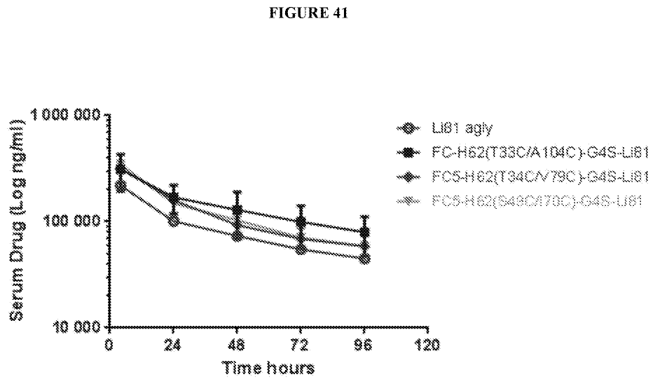

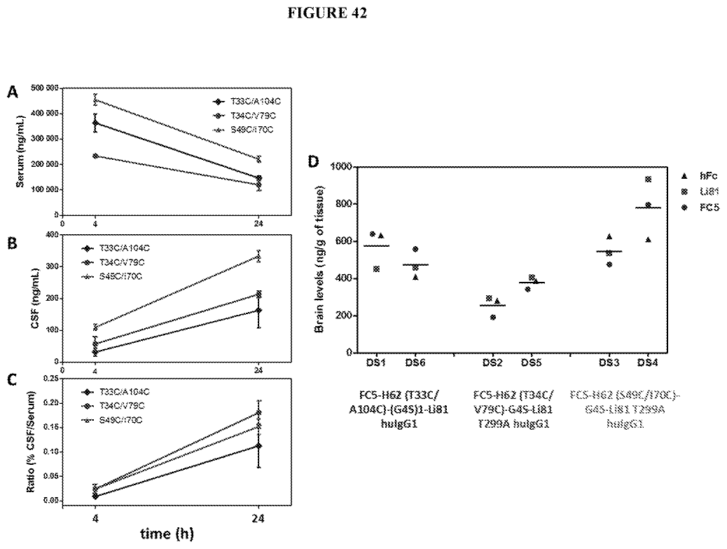

Disulfide stabilization led to about a 30% increase in brain levels. To test if other disulfide stabilized variants improved CNS exposure following systemic administration we tested H62-S49C/I70C (4D #4662), H62-T34C/V79C (4D #4665), H62-T33C/A104C (4D #4668) in side by side analysis. Results from this analysis are shown in FIG. 41 (serum analysis) and 42 (CSF and analysis of brain tissue). All three forms showed higher serum levels than Li81 and led to increased levels in CNS. These improvements translated into higher CSF and brain levels.

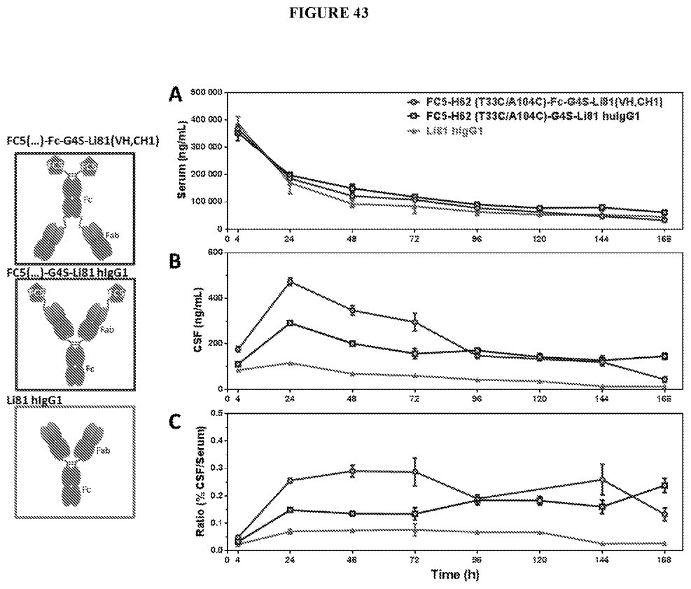

In our analysis of different framework designs in Example 1, we observed that both construct 3438, in which FC5 was attached to the N-terminus of Li81, and construct 3440, in which FC5 was attached to the Fc with a long hinge and the Li81 Fab was attached at the C-terminus of the Fc (Table 1, FIG. 11), showed good transport activity. As a result of this finding we engineered a disulfide stabilized version of the 3440 construct containing the FC5 H62 T33C/A104C design, in addition to the stabilized version of the 3438 construct described above (FIG. 40). PK analysis in rats of both samples shown in FIG. 43 revealed that both led improved CSF levels, with the FC5-H62(T33C/A104C)-Fc-G₄S-Li81(VH,CH1) (format 3440) showing higher levels over the first 72 h post IV administration and the FC5-H62(T33C/A104C)-G₄S-Li81 huIgG1 (format 3438) showing more modest but more sustained CSF levels over 168 h post IV administration. Schematics at the left of the panel show the two engineering designs.

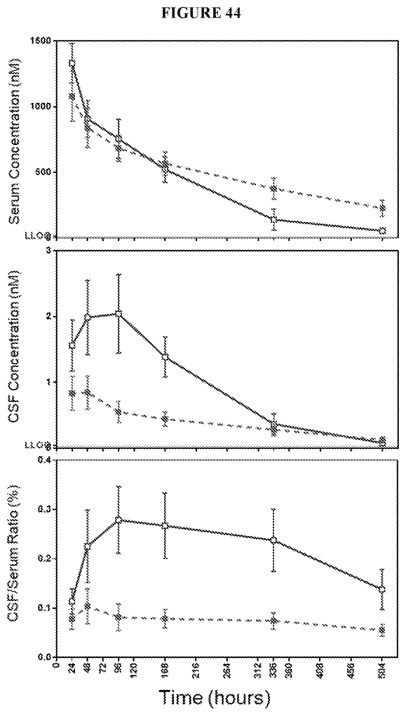

Further investigation of FC5-H62(T33C/A104C)-G₄S-Li81 (in the format of construct 3438) in cynomolgus monkey showed that the improved CSF exposure of this bispecific antibody, relative to the monospecific Li81, does translate to non-human primates. Monkeys were implanted with lumbar intrathecal catheters for repeated CSF collection and administered with 65 nmol/kg of antibody test articles via single IV bolus injection, with 12 naïve animals per group. Antibody levels in serum and CSF sample were quantified by nanoLC-MRM following heat-denaturation and tryptic digest, monitoring two peptides for Li81 and three peptides for FC5-H62(T33C/A104C)-G₄S-Li81 relative to protein standards in matching matrices. Results for serum and CSF antibody levels over a three-week period following IV administration are shown in FIG. 44. Serum PK was similar for both molecules, although a drop in serum drug levels of FC5-H62(T33C/A104C)-G₄S-Li81 compared to Li81 was observed in some animals >10 days after dosing. This corresponded to detection of anti-drug antibodies and a reduction in mean half-life (Table 13). CSF levels were elevated for FC5-H62(T33C/A104C)-G₄S-Li81 compared to Li81 over the first week post IV administration. CSF/serum ratios were elevated ˜3-fold for FC5-H62(T33C/A104C)-G₄S-Li81 over Li81, persisting from day 4 to 14 following IV administration. Non-compartmental pharmacokinetics analysis was performed using Pheonix 8.1 software and pharmacokinetic parameters are reported in Table 13.

TABLE 13
PK parameter estimates from noncompartmental analysis
of FC5-Li81 and Li81 serum and CSF concertation-
time profiles in cynomolgus monkeys

| | | | FC5-H62(T33C/A104C)- |
|---|---|---|---|
| Parameter | Unit | Li81 | G₄S-Li81 |
| Cₘₐₓ | nM | 1088 ± 125 | 1277 ± 167 |
| t_{1/2} | hr | 269 ± 69 | 106 ± 14 |
| AUC_{0-τ} | nmol*hr/mL | 266 ± 17 | 223 ± 20 |
| AUC_{0-∞} | nmol*hr/mL | 358 ± 40 | 232 ± 23 |
| CL | mL/hr/kg | 0.19 ± 0.02 | 0.30 ± 0.03 |
| CSF AUC_{0-τ}/ | % | 0.08 ± 0.01 | 0.21 ± 0.02 |
| serum AUC_{0-τ} | | | |

Cₘₐₓ: observed peak concentration in serum, t_{1/2}: half life in serum. AUC_{0-τ}: area under the serum concentration time curve from 0 to last measured concentration. AUC_{0-∞}: area under the serum concentration time curve from 0 to infinity. CL: total body clearance. Estimates are reported as mean ± sd.

To investigate whether the humanized and disulfide stabilized form of FC5 (FC5-H62(T33C/A104C); also referred to as hFC5.2) can increase brain endothelial cell permeability of other antibodies against brain targets, without interfering with binding to these targets, we generated fusions of hFC5.2 to anti-amyloid beta (12F6A), anti-alpha synuclein (12F4), and anti-tau (6C5) antibodies in different formats. All constructs were expressed in CHO and purified by Protein A affinity chromatography as described for the FC5-Li81 fusions. Size exclusion chromatography was used to remove aggregates and free heavy-chain or light-chain only contaminants in samples of molecules where FC5 was fused to the light chain.

For antibody 12F6A, FC5-H62(T33C/A104C) was fused to the N-terminus of the heavy chain via a G₄S linker (SEQ ID NO: 5) to form the FC5-H62(T33C/A104C)-12F6A construct.

### FC5-H62(T33C/A104C)-G₄S-12F6A

hreavy chain pYL1446
(SEQ ID NO: 34)
MGWSLILLFL VAVATRVLSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SGGGGSQVQL
VESGGGVVQP GRSLRLSCAA SGFAFSSYGM
HWVRQAPGKG LEWVAVIWFD GTKKYYTDSV
KGRFTISRDN SKNTLYLQMN TLRAEDTAVY
YCARDRGIGA RRGPYYMDVW GKGTTVTVSS
ASTKGPSVFP LAPSSKSTSG GTAALGCLVK
DYFPEPVTVS WNSGALTSGV HTFPAVLQSS
GLYSLSSVVT VPSSSLGTQT YICNVNHKPS
NTKVDKRVEP KSCDKTHTCP PCPAPELLGG
PSVFLFPPKP KDTLMISRTP EVTCVVVDVS
HEDPEVKFNW YVDGVEVHNA KTKPREEQYN
STYRVVSVLT VLHQDWLNGK EYKCKVSNKA
LPAPIEKTIS KAKGQPREPQ VYTLPPSREE
MTKNQVSLTC LVKGFYPSDI AVEWESNGQP
ENNYKTTPPV LDSDGSFFLY SKLTVDKSRW
QQGNVFSCSV MHEALHNHYT QKSLSLSPG
Light chain pJ0010
(SEQ ID NO: 35)
MDMRCPAQLL GLLLWFPGSR CDIQMIQSPS
SLSASVGDRV TITCRASQSI SSYLNWYQQK
PGKAPKLLIY AASSLQSGVP SRFSGSGSGT
DFTLTISSLQ PEDFATYYCQ QSYSTPLTFG
GGTKVEIKRT VAAPSVFIFP PSDEQLKSGT
ASVVCLLNNF YPREAKVQWK VDNALQSGNS
QESVTEQDSK DSTYSLSSTL TLSKADYEKH
KVYACEVTHQ GLSSPVTKSF NRGEC

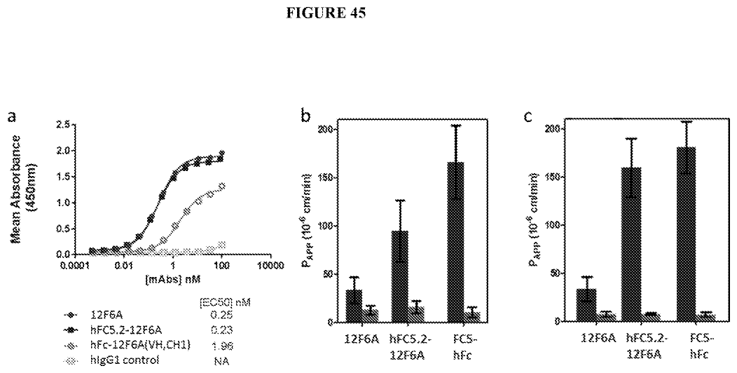

This fusion showed very similar binding to amyloid beta in an ELISA binding assay as the 12F6A antibody (FIG. 45A). A construct with the 12F6A Fab fused to the C-terminus of hIgG1 Fc showed significant loss in binding to amyloid beta, so FC5-H62(T33C/A104C) fusions were not pursued in this format. FC5-H62(T33C/A104C)-12F6A also showed increased penetration across rat brain endothelial cells in the SVARBEC transwell assay (FIG. 45B), with P_{APP} values 4-8 fold higher than a control antibody (anti-HEL murine IgG1; Goldbaum et al., J Immunol., 162:6040-6045 (1999); Genbank AF110316 VH and AY277254.1 VL). However, 12F6A alone also showed ˜˜2-fold higher P_{APP} than control, such that the P_{APP} for FC5-H62(T33C/A104C)-12F6A was ˜3-fold higher than 12F6A alone. Transcytosis on human brain endothelial cells was also demonstrated in a human pluripotent stem cell (hu-iPSC) derived transwell BBB model (Ribecco-Lutkiewicz et al. Sci Rep., 8(1):1873 (2018); FIG. 45C), in which significantly larger P_{APP} values were observed for FC5-H62(T33C/A104C)-12F6A (>10-fold over control antibody; ˜5-fold over 12F6A alone).

For antibody 12F4, fusion of FC5-H62(T33C/A104C) was made to the N-terminus of the light chain using a single G₄S linker (SEQ ID NO:5) to create FC5-H62(T33C/A104C)-12F4(LC), as the modification of the N-terminus of the heavy chain was previously determined to slightly reduce binding to alpha-synuclein.

Light chain pYL1593
(SEQ ID NO: 36)
MGWSLILLFL VAVATRVLSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SGGGGSSYEL
TQPPSVSVSP GQTARITCSG EALPMQFAHW
YQQRPGKAPV IVVYKDSERP SGVPERFSGS
SSGTTATLTI TGVQAEDEAD YYCQSPDSTN
TYEVFGGGTK LTVLSQPKAA PSVTLFPPSS
EELQANKATL VCLISDFYPG AVTVAWKADS
SPVKAGVETT TPSKQSNNKY AASSYLSLTP
EQWKSHRSYS CQVTHEGSTV EKTVAPTECS

To investigate whether FC5-based transcytosis is affected by differences in the Fc region, which modulate binding to Fc receptors, FC5-H62(T33C/A104C)-12F4(LC) was co-expressed with 12F4 heavy chains with wild type hIgG1 subclass as well as three scaffolds with reduced effector function: hIgG1 agly (N297Q), hIgG2 SAA (see, Vafa et al. Methods. 65(1):114-26 (2014), and hIgG4P/G1 agly (see, US 2012/0100140 A1).

12F4 hIgG1 (HC)
(SEQ ID NO: 29)
EVQLVESGGGLVEPGGSLRLSCAVSGFDFEKAWMS
WVRQAPGQGLQWVARIKSTADGGTTSYAAPVEGRF
IISRDDSRNMLYLQMNSLKTEDTAVYYCTSAHWGQ
GTLVTVSSASTKGPSVFPLAPSSKSTSGGTAALGC
LVKDYFPEPVTVSWNSGALTSGVHTFPAVLQSSGL
YSLSSVVTVPSSSLGTQTYICNVNHKPSNTKVDKR
VEPKSCDKTHTCPPCPAPELLGGPSVFLFPPKPKD
TLMISRTPEVTCVVVDVSHEDPEVKFNWYVDGVEV
HNAKTKPREEQYNSTYRVVSVLTVLHQDWLNGKEY
KCKVSNKALPAPIEKTISKAKGQPREPQVYTLPPS
REEMTKNQVSLTCLVKGFYPSDIAVEWESNGQPEN
NYKTTPPVLDSDGSFFLYSKLTVDKSRWQQGNVFS
CSVMHEALHNHYTQKSLSLSPG
12F4 hIgG1 agly
(SEQ ID NO: 30)
EVQLVESGGGLVEPGGSLRLSCAVSGFDFEKAWMS
WVRQAPGQGLQWVARIKSTADGGTTSYAAPVEGRF
IISRDDSRNMLYLQMNSLKTEDTAVYYCTSAHWGQ
GTLVTVSSASTKGPSVFPLAPSSKSTSGGTAALGC
LVKDYFPEPVTVSWNSGALTSGVHTFPAVLQSSGL
YSLSSVVTVPSSSLGTQTYICNVNHKPSNTKVDKR
VEPKSCDKTHTCPPCPAPELLGGPSVFLFPPKPKD
TLMISRTPEVTCVVVDVSHEDPEVKFNWYVDGVEV
HNAKTKPREEQWSTYRVVSVLTVLHQDWLNGKEYK
CKVSNKALPAPIEKTISKAKGQPREPQVYTLPPSR
EEMTKNQVSLTCLVKGFYPSDIAVEWESNGQPENN
YKTTPPVLDSDGSFFLYSKLTVDKSRWQQGNVFSC
SVMHEALHNHYTQKSLSLSPG
12F4 hIgG2 SAA
(SEQ ID NO: 31)
EVQLVESGGGLVEPGGSLRLSCAVSGFDFEKAWMS
WVRQAPGQGLQWVARIKSTADGGTTSYAAPVEGRF
IISRDDSRNMLYLQMNSLKTEDTAVYYCTSAHWGQ
GTLVTVSSASTKGPSVFPLAPCSRSTSESTAALGC
LVKDYFPEPVTVSWNSGALTSGVHTFPAVLQSSGL
YSLSSVVTVPSSNFGTQTYTCNVDHKPSNTKVDKT
VERKCVECPPCPAPPAAAPSVFLFPPKPKDTLMIS
RTPEVTCVVVDVSHEDPEVQFNWYVDGVEVHNAKT
KPREEQFNSTFRVVSVLTVVHQDWLNGKEYKCKVS
NKGLPAPIEKTISKTKGQPREPQVYTLPPSREEMT
KNQVSLTCLVKGFYPSDIAVEWESNGQPENNYKTT
PPMLDSDGSFFLYSKLTVDKSRWQQGNVFSCSVMH
EALHNHYTQKSLSLSPG
12F4 hIgG4P/G1
(SEQ ID NO: 32)
EVQLVESGGGLVEPGGSLRLSCAVSGFDFEKAWMS
WVRQAPGQGLQWVARIKSTADGGTTSYAAPVEGRF
IISRDDSRNMLYLQMNSLKTEDTAVYYCTSAHWGQ
GTLVTVSSASTKGPSVFPLAPCSRSTSESTAALGC
LVKDYFPEPVTVSWNSGALTSGVHTFPAVLQSSGL
YSLSSVVTVPSSSLGTKTYTCNVDHKPSNTKVDKR
VESKYGPPCPPCPAPEFLGGPSVFLFPPKPKDTLM
ISRTPEVTCVVVDVSQEDPEVQFNWYVDGVEVHNA
KTKPREEQFSTYRVVSVLTVLHQDWLNGKEYKCKV
SNKGLPSSIEKTISKAKGQPREPQVYTLPPSRDEL
TKNQVSLTCLVKGFYPSDIAVEWESNGQPENNYKT
TPPVLDSDGSFFLYSKLTVDKSRWQQGNVFSCSVM
HEALHNHYTQKSLSLSPG

In addition, a construct with FC5-H62(T33C/A104C) fused to long-hinge hIgG1 Fc and the 12F4 Fab through G₄S linkage from the C-terminus of Fc to the 12F4 light chain (FC5-H62(T33C/A104C)-Fc-12F4 Fab(LC) was generated.

FC5-H62(T33C/A104C)-Fc-12F4
Fab(LC), pYL1607
(SEQ ID NO: 37)
MGWSLILLFL VAVATRVLSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SAEPKSCDKT
HTCPPCPAPE LLGGPSVFLF PPKPKDTLMI
SRTPEVTCVV VDVSHEDPEV KFNWYVDGVE
VHNAKTKPRE EQYNSTYRVV SVLTVLHQDW
LNGKEYKCKV SNKALPAPIE KTISKAKGQP
REPQVYTLPP SREEMTKNQV SLTCLVKGFY
PSDIAVEWES NGQPENNYKT TPPVLDSDGS
FFLYSKLTVD KSRWQQGNVF SCSVMHEALH
NHYTQKSLSL SPGGGGGSSY ELTQPPSVSV
SPGQTARITC SGEALPMQFA HWYQQRPGKA
PVIVVYKDSE RPSGVPERFS GSSSGTTATL
TITGVQAEDE ADYYCQSPDS TNTYEVFGGG
TKLTVLSQPK AAPSVTLFPP SSEELQANKA
TLVCLISDFY PGAVTVAWKA DSSPVKAGVE
TTTPSKQSNN KYAASSYLSL TPEQWKSHRS
YSCQVTHEGS TVEKTVAPTE CS
12F4W(VH-VH1) pYL1595
(SEQ ID NO: 38)
MDMRVPAQLL GLLLLWFPGS RCEVQLVESG
GGLVEPGGSL RLSCAVSGFD FEKAWMSWVR
QAPGQGLQWV ARIKSTADGG TTSYAAPVEG
RFIISRDDSR NMLYLQMNSL KTEDTAVYYC
TSAHWGQGTL VTVSSASTKG PSVFPLAPSS
KSTSGGTAAL GCLVKDYFPE PVTVSWNSGA
LTSGVHTFPA VLQSSGLYSL SSVVTVPSSS
LGTQTYICNV NHKPSNTKVD KRVEPKSC

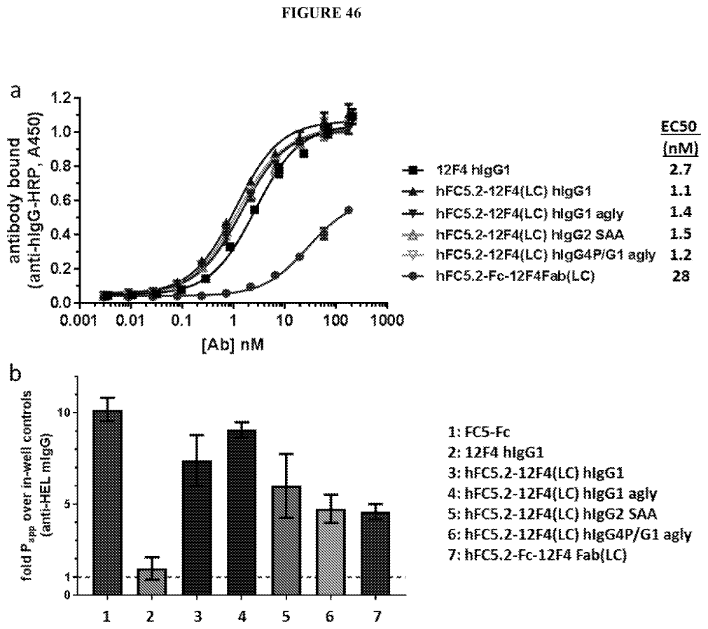

Analysis of these molecules by alpha-synuclein direct binding ELISA showed that the FC5-H62(T33C/A104C) domain fused directly to the 12F4 light chain does not impair binding to alpha-synuclein, compared to the binding of 12F4 hIgG1 (FIG. 46A). In contrast, the FC5-H62(T33C/A104C)-Fc-12F4 Fab(LC) molecule showed severely impaired binding to alpha-synuclein. The series of effector function variants of FC5-H62(T33C/A104C)-12F4(LC) hIgG molecules were further tested for functional transcytosis in the rat brain endothelial cell (SVARBEC) transwell assay (FIG. 46B). All molecules, regardless of the Fc framework, showed improved transport, with P_{APP} values that were 4- to 9-fold higher than an anti-HEL mIgG1 control antibody included in each transwell, whereas 12F4 without FC5 attached did not show significant difference in trans-cell permeability from control.

To identify a bifunctional anti-tau binding antibody with enhanced BBB penetration, FC5-H62(T33C/A104C)-6C5 hIgG1 was generated both as heavy chain and light chain fusion molecules.

FCS-H62(T33C/A104C)-G4S-6C5 hIgG1 (HC)
Heavy Chain pYL1611
(SEQ ID NO: 39)
MGWSLILLFL VAVATRVLSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SGGGGSQVQL
VESGGGVVQP GRSLRVSCAA SGFTFSSYDM
HWVRQAPGKG LEWVAVIWFD GSNEFYADSV
KGRFTISRDN SKNTLFLQMN SLRAEDTAVY
YCARDLGASV TTSNAENFHH WGQGTLVTVS
SASTKGPSVF PLAPSSKSTS GGTAALGCLV
KDYFPEPVTV SWNSGALTSG VHTFPAVLQS
SGLYSLSSVV TVPSSSLGTQ TYICNVNHKP
SNTKVDKRVE PKSCDKTHTC PPCPAPELLG
GPSVFLFPPK PKDTLMISRT PEVTCVVVDV
SHEDPEVKFN WYVDGVEVHN AKTKPREEQY
NSTYRVVSVL TVLHQDWLNG KEYKCKVSNK
ALPAPIEKTI SKAKGQPREP QVYTLPPSRE
EMTKNQVSLT CLVKGFYPSD IAVEWESNGQ
PENNYKTTPP VLDSDGSFFL YSKLTVDKSR
WQQGNVFSCS VMHEALHNHY TQKSLSLSPG
Light chain, pEAG2942
(SEQ ID NO: 40)
MDMRVPAQLL GLLLLWFPGS RCSYELTQPP
SVSVSPGQTA RITCSGDALP KRYVYWYQQK
SGQAPVLVIY EDSKRPSGIP ETFSGSSSGT
MATLTISGAQ VEDEADYYCY STDSNGHHWV
FGGGTKLTVL GQPKAAPSVT LFPPSSEELQ
ANKATLVCLI SDFYPGAVTV AWKADSSPVK
AGVETTTPSK QSNNKYAASS YLSLTPEQWK
SHRSYSCQVT HEGSTVEKTV APTECS
FCS-H62(T33C/A104C)-G4S-6C5 (LC)
Light chain, pYL1613
(SEQ ID NO: 41)
MGWSLILLFL VAVATRVLSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SGGGGSSYEL
TQPPSVSVSP GQTARITCSG DALPKRYVYW
YQQKSGQAPV LVIYEDSKRP SGIPETFSGS
SSGTMATLTI SGAQVEDEAD YYCYSTDSNG
HHWVFGGGTK LTVLGQPKAA PSVTLFPPSS
EELQANKATL VCLISDFYPG AVTVAWKADS
SPVKAGVETT TPSKQSNNKY AASSYLSLTP
EQWKSHRSYS CQVTHEGSTV EKTVAPTECS
Heavy chain, pEAG2974
(SEQ ID NO: 42)
MDMRVPAQLL GLLLLWFPGSS RCQVQLVESG
GGVVQPGRSL RVSCAASGFT FSSYDMHWVR
QAPGKGLEWV AVIWFDGSNE FYADSVKGRF
TISRDNSKNT LFLQMNSLRA EDTAVYYCAR
DLGASVTTSN AENFHHWGQG TLVTVSSAST
KGPSVFPLAP SSKSTSGGTA ALGCLVKDYF
PEPVTVSWNS GALTSGVHTF PAVLQSSGLY
SLSSVVTVPS SSLGTQTYIC NVNHKPSNTK
VDKRVEPKSC DKTHTCPPCP APELLGGPSV
FLFPPKPKDT LMISRTPEVT CVVVDVSHED
PEVKFNWYVD GVEVHNAKTK PREEQYNSTY
RVVSVLTVLH QDWLNGKEYK CKVSNKALPA
PIEKTISKAK GQPREPQVYT LPPSREEMTK
NQVSLTCLVK GFYPSDIAVE WESNGQPENN
YKTTPPVLDS DGSFFLYSKL TVDKSRWQQG
NVFSCSVMHE ALHNHYTQKS LSLSPG

Also, the alternate format FC5-H62(T33C/A104C)-Fc-6C5 Fab was generated as heavy chain and light chain fusion molecules.

FCS-H62(T33C/A104C)-Fc-6C5 Fab(HC)
Heavy chain, pYL1612
(SEQ ID NO: 43)
MGWSLTLLFL VAVTRVLDSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SAEPKSCDKT
HTCPPCPAPE LLGGPSVFLF PPKPKDTLMI
SRTPEVTCVV VDVSHEDPEV KFNWYVDGVE
VHNAKTKPRE EQYNSTYRVV SVLTVLHQDW
LNGKEYKCKV SNKALPAPIE KTISKAKGQP
REPQVYTLPP SREEMTKNQV SLTCLVKGFY
PSDIAVEWES NGQPENNYKT TPPVLDSDGS
FFLYSKLTVD KSRWQQGNVF SCSVMHEALH
NHYTQKSLSL SPGGGGGSQV QLVESGGGVV
QPGRSLRVSC AASGFTFSSY DMHWVRQAPG
KGLEWVAVIW FDGSNEFYAD SVKGRFTISR
DNSKNTLFLQ MNSLRAEDTA VYYCARDLGA
SVTTSNAENF HHWGQGTLVT VSSASTKGPS
VFPLAPSSKS TSGGTAALGC LVKDYFPEPV
TVSWNSGALT SGVHTFPAVL QSSGLYSLSS
VVTVPSSSLG TQTYICNVNH KPSNTKVDKR
VEPKSC
Light chain, pEAG2942
(SEQ ID NO: 44)
MDMRVPAQLL GLLLLWFPGS RCSYELTQPP
SVSVSPGQTA RITCSGDALP KRYVYWYQQK
SGQAPVLVIY EDSKRPSGIP ETFSGSSSGT
MATLTISGAQ VEDEADYYCY STDSNGHHWV
FGGGTKLTVL GQPKAAPSVT LFPPSSEELQ
ANKATLVCLI SDFYPGAVTV AWKADSSPVK
AGVETTTPSK QSNNKYAASS YLSLTPEQWK
SHRSYSCQVT HEGSTVEKTV APTECS
FCS-H62(T33C/A104C)-Fc-6C5 Fab(LC)
pYL1614
(SEQ ID NO: 45)
MGWSLILLFL VAVTRVLDSD VQLVESGGGL
VQPGGSLRLS CAASGFKITH YCTGWFRQAP
GKEREFVSRI TWGGDNTFYS NSVKGRFTIS
RDNSKNTVYL QMNSLRAEDT AVYYCAAGST
STCTPLRVDY WGQGTLVTVS SAEPKSCDKT
HTCPPCPAPE LLGGPSVFLF PPKPKDTLMI
SRTPEVTCVV VDVSHEDPEV KFNWYVDGVE
VHNAKTKPRE EQYNSTYRVV SVLTVLHQDW
LNGKEYKCKV SNKALPAPIE KTISKAKGQP
REPQVYTLPP SREEMTKNQV SLTCLVKGFY
PSDIAVEWES NGQPENNYKT TPPVLDSDGS
FFLYSKLTVD KSRWQQGNVF SCSVMHEALH
NHYTQKSLSL SPGGGGGSSY ELTQPPSVSV
SPGQTARITC SGDALPKRYV YWYQQKSGQA
PVLVIYEDSK RPSGIPETFS GSSSGTMATL
TISGAQVEDE ADYYCYSTDS NGHHWVFGGG
TKLTVLGQPK AAPSVTLFPP SSEELQANKA
TLVCLISDFY PGAVTVAWKA DSSPVKAGVE
TTTPSKQSNN KYAASSYLSL TPEQWKSHRS
YSCQVTHEGS TVEKTVAPTE CS
pYL1596
(SEQ ID NO: 46)
MDMRVPAQLL GLLLLWFPGS RCQVQLVESG
GGVVQPGRSL RVSCAASGFT FSSYDMHWVR
QAPGKGLEWV AVIWFDGSNE FYADSVKGRF
TISRDNSKNT LFLQMNSLRA EDTAVYYCAR
DLGASVTTSN AENFHHWGQG TLVTVSSAST
KGPSVFPLAP SSKSTSGGTA ALGCLVKDYF
PEPVTVSWNS GALTSGVHTF PAVLQSSGLY
SLSSVVTVPS SSLGTQTYIC NVNHKPSNTK
VDKRVEPKSC

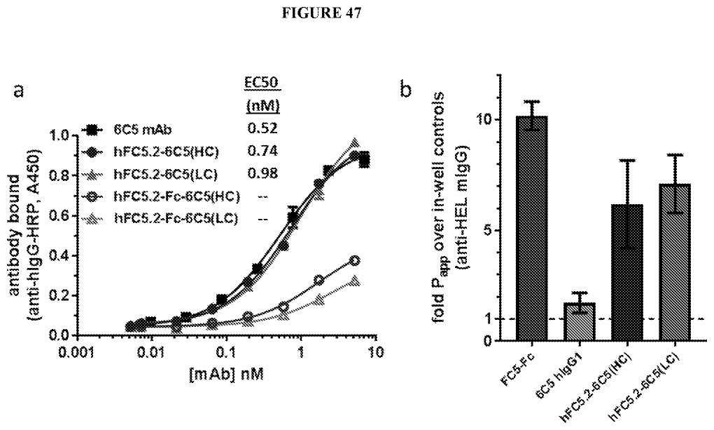

FC5-H62(T33C/A104C)-6C5 hIgG1 showed similar binding to tau as 6C5 hIgG1 (FIG. 47A), whereas FC5-H62(T33C/A104C)-Fc-6C5 Fab molecules showed significantly reduced tau binding. FC5-H62(T33C/A104C)-6C5(HC) hIgG1 and FC5-H62(T33C/A104C)-6C5(LC) hIgG1 molecules were further tested for transcytosis in the SVARBEC transwell assay (FIG. 47B), and showed 4- to 8-fold higher P_{APP} than control antibody. 6C5 hIgG1 showed slightly higher P_{APP} than control (1.7-fold over anti-HEL mIgG1).

To validate that disulfide engineered FC5, fused to 12F4 and 12F6A antibodies, has improved molecular stability over non-disulfide engineered variants, as was shown for disulfide engineered FC5-H62(T33C/A104C)-Li81 (FIG. 35), pepsin digest was performed. FC5-H62(T33C/A104C)-Li81 hIgG1 agly was used as an example of a molecule with good stability against pepsin digest, FC5-H32-Li81 hIgG1 agly was used as an example of a molecule with poor stability, and pepsin digests of these were compared to digests of disulfide engineered FC5-H62(T33C/A104C)-12F4 (on both wild type and aglycosylated hIgG1 scaffolds), FC5-H62(T33C/A104C)-12F6A hIgG1, and non-stabilized FC5-H32-12F6A hIgG1 (FIG. 48). All protein digests were performed in 20 mM sodium acetate, pH 3.6 at 1 mg/mL and 0.033 mg/mL pepsin, incubated at 37° C. for either 1 h or 2.5 h. All molecules with the additional disulfide (FC5-H62(T33C/A104C) fusions) showed higher intensity of the higher molecular weight bands by SDS-PAGE analysis following digest as compared to the molecules that were not disulfide engineered (FC5-H32 fusions). A reduction in the lower molecular weight bands, highlighted with arrows in FIG. 48, was observed for the FC5-H32 fusions, is a positive control for protease sensitivity without disulfide stabilization.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody variable domain that transmigrates across the blood brain barrier, wherein the antibody variable domain comprises an amino acid sequence that is at least 85% identical to the sequence set forth in SEQ ID NO:1, wherein the amino acid sequence comprises:
(a) complementarity determining region (CDR)1, CDR2, and CDR3, wherein CDR2 comprises the sequence set forth in SEQ ID NO:50, and wherein:
(i) CDR1 comprises the sequence set forth in SEQ ID NO:47, CDR3 comprises the sequence set forth in SEQ ID NO:51, and the amino acid sequence comprises cysteines at the positions corresponding to positions 49 and 70 of SEQ ID NO:1;
(ii) CDR1 comprises the sequence set forth in SEQ ID NO:48, CDR3 comprises the sequence set forth in SEQ ID NO:51, and the amino acid sequence comprises cysteine at the position corresponding to position 79 of SEQ ID NO:1;
(iii) CDR1 comprises the sequence set forth in SEQ ID NO:49, and CDR3 comprises the sequence set forth in SEQ ID NO:52;
(iv) CDR1 comprises the sequence set forth in SEQ ID NO:58, and CDR3 comprises the sequence set forth in SEQ ID NO:52;
(v) CDR1 comprises the sequence set forth in SEQ ID NO:49, and CDR3 comprises the sequence set forth in SEQ ID NO:53;
(vi) CDR1 comprises the sequence set forth in SEQ ID NO:58, and CDR3 comprises the sequence set forth in SEQ ID NO:53;
(vii) CDR1 comprises the sequence set forth in SEQ ID NO:49, and CDR3 comprises the sequence set forth in SEQ ID NO:54;
(viii) CDR1 comprises the sequence set forth in SEQ ID NO:58, and CDR3 comprises the sequence set forth in SEQ ID NO:54;
(ix) CDR1 comprises the sequence set forth in SEQ ID NO:49, and CDR3 comprises the sequence set forth in SEQ ID NO:55; or
(x) CDR1 comprises the sequence set forth in SEQ ID NO:58, and CDR3 comprises the sequence set forth in SEQ ID NO:55;
or
(b) (i) amino acid substitutions, as compared to SEQ ID NO:1, at one or more of the positions corresponding to positions 5, 6, 14, 75, 87, 88, 93, 114, and 117 of the sequence set forth in SEQ ID NO:1; and (ii) CDR1 comprising the sequence set forth in SEQ ID NO:47 or 57, CDR2 comprising the sequence set forth in SEQ ID NO:50, and CDR3 comprising the sequence set forth in SEQ ID NO:51.

2.-23. (canceled)

24. The antibody variable domain of claim 1, wherein the amino acid sequence of part (b) comprises amino acid substitutions, as compared to SEQ ID NO:1, at at least four, at least five, at least six, or at each of the positions corresponding to positions 5, 6, 14, 75, 87, 88, 93, 114, and 117 of the sequence set forth in SEQ ID NO:1.

25.-47. (canceled)

48. The antibody variable domain of claim 1, wherein the amino acid sequence of part (b) is the sequence set forth in any one of SEQ ID NOs:10 to 17.

49.-50. (canceled)

51. A chimeric molecule comprising the antibody variable domain of claim 1.

52. The chimeric molecule of claim 51, comprising an antibody Fc region.

53. The chimeric molecule of claim 51, comprising an antibody, an antigen-binding fragment of an antibody, a peptide, an enzyme, an oligonucleotide, a small molecule drug, or a liposome or a lipid nanoparticle encapsulating a nucleic acid, small molecule drug, or peptide.

54. The chimeric molecule of claim 51, wherein the antibody variable domain is linked
(i) directly, or
(ii) via an intervening amino acid sequence,
to the N-terminus of a hinge region of an antibody, and wherein the hinge region is fused to an agent selected from the group consisting of:
(i) an Fc moiety;
(ii) a liposome or lipid nanoparticle;
(iii) a whole antibody; and
(iv) a viral capsid containing a therapeutic vector.

55.-57. (canceled)

58. The chimeric molecule of claim 54, wherein the agent is linked directly or via a second intervening amino acid sequence to a polypeptide comprising a second antibody variable domain; an Fab; an scFv; a single domain antibody; a peptide; an enzyme; a nucleic acid; or an oligonucleotide.

59.-66. (canceled)

67. A composition comprising:
(I)(1) a chimeric protein that comprises (i) the antibody variable domain of claim 1 and (ii) a first protein comprising a first hinge region of an antibody and a first Fc moiety, wherein the C-terminus of the first hinge region is linked to the N-terminus of the first Fc moiety, and wherein the C-terminus of the antibody variable domain is fused directly or via an intervening amino acid sequence to the N-terminus of the first hinge region; (2) a second protein comprising a second hinge region of an antibody and a second Fc moiety, wherein the C-terminus of the second hinge region is linked to the N-terminus of the second Fc moiety, wherein the second protein pairs with the first protein; and (3) a therapeutic agent;
(II)(1) a first chimeric protein that comprises (i) a first antibody variable domain of claim 1; and (ii) a first protein comprising a first hinge region of an antibody and a first Fc moiety, wherein the C-terminus of the first hinge region is linked to the N-terminus of the first Fc moiety, and wherein the C-terminal of the first antibody variable domain is fused directly or via an intervening amino acid sequence to the N-terminus of the first hinge region; (2) a second chimeric protein that comprises (i) a second antibody variable domain of claim 1; and (ii) a second protein comprising a second hinge region of an antibody and a second Fc moiety, wherein the C-terminus of the second hinge region is linked to the N-terminus of the second Fc moiety, wherein the C-terminal of the second antibody variable domain is fused directly or via an intervening amino acid sequence to the N-terminus of the second hinge region, and wherein the second protein pairs with the first protein; and (3) a therapeutic agent or
(III)(1) a first chimeric protein that comprises (i) a first antibody variable domain of claim 1; and (ii) a first protein comprising a first hinge region of an antibody and a first Fc moiety, wherein the C-terminus of the first hinge region is linked to the N-terminus of the first Fc moiety, and wherein the C-terminal of the first antibody variable domain is fused via a first intervening amino acid sequence to the N-terminus of the first hinge region; and (2) a second chimeric protein that comprises (i) a second antibody variable domain of claim 1; and (ii) a second protein comprising a second hinge region of an antibody and a second Fc moiety, wherein the C-terminus of the second hinge region is linked to the N-terminus of the second Fc moiety, wherein the C-terminal of the second antibody variable domain is fused via a second intervening amino acid sequence to the N-terminus of the second hinge region, and wherein the second protein pairs with the first protein; wherein the first and second intervening amino acid sequence are Fabs that act as therapeutic agents;
wherein the first and second antibody variable domains of (II)-(III), are the antibody variable domain of claim 1.

68.-71. (canceled)

72. The composition of claim 67, wherein (a) the first and second Fc moieties have an identical amino acid sequence or different amino acid sequences; (b) the first and second hinge regions have an identical amino acid sequence and/or (c) the therapeutic agent is a binding molecule that comprises an antibody variable domain or an antisense oligonucleotide.

73.-81. (canceled)

82. A composition comprising:
(I)(1) a chimeric protein comprising (i) the antibody variable domain of claim 1, and (ii) a light chain of an antibody, wherein the C-terminus of the antibody variable domain is fused directly or via an intervening amino acid sequence to the N-terminus of the light chain of the antibody; and (2) a heavy chain of the antibody that pairs with the light chain of the antibody;
(II) (1) a chimeric protein comprising (i) the antibody variable domain of claim 1; and (ii) a heavy chain of an antibody, wherein the C-terminus of the antibody variable domain is fused directly or via an intervening amino acid sequence to the N-terminus of the heavy chain of the antibody; and (2) a light chain of the antibody that pairs with the heavy chain of the antibody; or
(III) (1) a first chimeric protein comprising (i) a first antibody variable domain of claim 1, and (ii) a light chain of an antibody, wherein the C-terminus of the first antibody variable domain is fused directly or via an intervening amino acid sequence to the N-terminus of the light chain of the antibody; and (2) a second chimeric protein comprising (i) a second antibody variable domain of claim 1, and (ii) a heavy chain of the antibody, wherein the C-terminus of the second antibody variable domain is fused directly or via an intervening amino acid sequence to the N-terminus of the heavy chain of the antibody;
wherein the first and second antibody variable domains of (III), are the antibody variable domain of claim 1.

83.-85. (canceled)

86. A composition comprising a chimeric protein comprising (i) the antibody variable domain of claim 1, (ii) a protein comprising a hinge region of an antibody and an Fc moiety, wherein the C-terminus of the antibody variable domain is fused directly or via an intervening amino acid sequence to the N-terminus of the hinge region of the protein, and (iii) a therapeutic agent.

87.-98. (canceled)

99. A pharmaceutical composition comprising the antibody variable domain of claim 1, and a pharmaceutically acceptable carrier.

100. A nucleic acid encoding the antibody variable domain of claim 1.

101. A vector comprising the nucleic acid of claim 100.

102. A host cell comprising the vector of claim 101.

103. A method of producing an antibody variable domain, the method comprising:
culturing the host cell of claim 102 in a cell culture medium under conditions that result in the expression of the antibody variable domain; and
isolating the antibody variable domain from the cell culture medium.

104. A method of treating Alzheimer's disease in a human subject in need thereof, the method comprising administering to the subject the antibody variable domain of claim 1 linked directly or via an intervening amino acid sequence to an antibody or antigen-binding fragment thereof that specifically binds human beta-amyloid.

105.-109. (canceled)

110. A method of treating a synucleinopathy in a human subject in need thereof, the method comprising administering to the subject the antibody variable domain of claim 1 linked directly or via an intervening amino acid sequence to an antibody or antigen-binding fragment thereof that specifically binds human alpha synuclein.

111.-115. (canceled)

116. A method of treating a tauopathy in a human subject in need thereof, the method comprising administering to the subject the antibody variable domain of claim 1 linked directly or via an intervening amino acid sequence to an antibody or antigen-binding fragment thereof that specifically binds human tau.

117.-121. (canceled)

122. A method of treating frontotemporal dementia in a human subject in need thereof, the method comprising administering to the subject the antibody variable domain of claim 1 linked directly or via an intervening amino acid sequence to an antibody or antigen-binding fragment thereof that specifically binds human TDP-43.

123.-127. (canceled)

128. A method of treating multiple sclerosis in a human subject in need thereof, the method comprising administering to the subject the antibody variable domain of claim 1 linked directly or via an intervening amino acid sequence to an antibody or antigen-binding fragment thereof that specifically binds human LINGO-1.

129.-133. (canceled)

134. A method of treating spinal muscular atrophy in a human subject in need thereof, the method comprising administering to the subject the antibody variable domain of claim 1 linked directly or via an intervening amino acid sequence to nusinersen.

135.-136. (canceled)

137. A method of assessing the lability of an antibody variable domain, the method comprising
providing an antibody variable domain;
adding the antibody variable domain to a serum sample to create a mixture;
incubating the mixture;
purifying the antibody variable domain; and
performing peptide mapping.

138.-142. (canceled)

143. A method of screening for a stabilized form of FCS, the method comprising:
providing an antibody variable domain comprising a FC5 variant that differs from SEQ ID NO:1 at one or more amino acids;
adding the antibody variable domain to a serum sample to create a mixture;
incubating the mixture;
purifying the antibody variable domain;
performing peptide mapping; and
selecting an antibody variable domain that exhibits increased peptide recovery.

144.-147. (canceled)

148. A nucleic acid encoding the chimeric molecule of claim 51.

149. A vector comprising the nucleic acid of claim 148.

150. A host cell comprising the vector of claim 149.

151. A method of producing a chimeric molecule, the method comprising:
culturing the host cell of claim 150 in a cell culture medium under conditions that result in the expression of the chimeric molecule; and
isolating the chimeric molecule from the cell culture medium.

152. A nucleic acid encoding the composition of claim 67.

153. A vector comprising the nucleic acid of claim 152.

154. A host cell comprising the vector of claim 153.

155. A method of producing a composition, the method comprising:
culturing the host cell of claim 154 in a cell culture medium under conditions that result in the expression of the composition; and
isolating the composition from the cell culture medium.

156.-157. (canceled)

158. A pharmaceutical composition comprising the chimeric molecule of claim 51, and a pharmaceutically acceptable carrier.

159. A pharmaceutical composition comprising the composition of claim 67, and a pharmaceutically acceptable carrier.


SEQUENCE LISTING (204 sequences)

SEQ ID NO: 1 - protein, 122 aa, Unknown
  source: /note=“Description of Unknown: wild type FC5 single domain antibody sequence”
DVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 2 - protein, 237 aa, Artificial Sequence
  source: /note=“Description of Artificial Sequence: Synthetic polypeptide”
MDMRVPAQLLGLLLLWLRGARCDIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 603 aa, Artificial Sequence
  source: /note=“Description of Artificial Sequence: Synthetic polypeptide”
MGWSLILLFLVAVATRVLSDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGGSGGGGSGGGGSEVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 371 aa, Artificial Sequence
  source: /note=“Description of Artificial Sequence: Synthetic polypeptide”
MGWSLILLFLVAVATRVLSDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGGSGGGGSGGGGSDIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Artificial Sequence
  source: /note=“Description of Artificial Sequence: Synthetic peptide”
GGGGS

SEQ ID NO: 6 - protein, 15 aa, Artificial Sequence
  source: /note=“Description of Artificial Sequence: Synthetic peptide”
GGGGSGGGGSGGGGS

SEQ ID NO: 7 - protein, 593 aa, Artificial Sequence
  source: /note=“Description of Artificial Sequence: Synthetic polypeptide”
MGWSLILLFLVAVATRVLSDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGGSEVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 600 aa, Artificial Sequence
  source: /note=“Description of Artificial Sequence: Synthetic polypeptide”
MDWTWRVFCLLAVAPGAHSDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSAEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 593 aa, Artificial Sequence
  source: /note=“Description of Artificial Sequence: Synthetic polypeptide”
MDWTWRVFCLLAVAPGAHSDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 122 aa, Artificial Sequence
  source: /note=“Description of Artificial Sequence: Synthetic polypeptide”
EVQLVESGGGLVQPGGSLRLSCAASGFKITHYTMGWVRQAPGKGLEWVSRITWGGDNTFYSNSVKGRFTISRDNSKNTLYLQMNSLRAEDTAVYYCAAGSTSTATPLRVDYWGQGTLVTVSS

SEQ ID NO: 11 - protein, 122 aa, Artificial Sequence
  source: /note=“Description of Artificial Sequence: Synthetic polypeptide”
EVQLVESGGGLVQPGGSLRLSCAASGFKITHYTTGWVRQAPGKGLEWVSRITWGGDNTFYSNSVKGRFTISRDNSKNTLYLQMNSLRAEDTAVYYCAAGSTSTATPLRVDYWGQGTLVTVSS

SEQ ID NO: 12 - protein, 122 aa, Artificial Sequence
  source: /note=“Description of Artificial Sequence: Synthetic polypeptide”
EVQLVESGGGLVQPGGSLRLSCAASGFKITHYTMGWFRQAPGKGLEFVSRITWGGDNTFYSNSVKGRFTISRDNSKNTVYLQMNSLRAEDTAVYYCAAGSTSTATPLRVDYWGQGTLVTVSS

SEQ ID NO: 13 - protein, 122 aa, Artificial Sequence
  source: /note=“Description of Artificial Sequence: Synthetic polypeptide”
EVQLVESGGGLVQPGGSLRLSCAASGFKITHYTTGWFRQAPGKGLEFVSRITWGGDNTFYSNSVKGRFTISRDNSKNTVYLQMNSLRAEDTAVYYCAAGSTSTATPLRVDYWGQGTLVTVSS

SEQ ID NO: 14 - protein, 122 aa, Artificial Sequence
  source: /note=“Description of Artificial Sequence: Synthetic polypeptide”
DVQLVESGGGLVQPGGSLRLSCAASGFKITHYTMGWFRQAPGKEREFVSRITWGGDNTFYSNSVKGRFTISRDNSKNTVYLQMNSLRAEDTAVYYCAAGSTSTATPLRVDYWGQGTLVTVSS

SEQ ID NO: 15 - protein, 122 aa, Artificial Sequence
  source: /note=“Description of Artificial Sequence: Synthetic polypeptide”
DVQLVESGGGLVQPGGSLRLSCAASGFKITHYTTGWFRQAPGKEREFVSRITWGGDNTFYSNSVKGRFTISRDNSKNTVYLQMNSLRAEDTAVYYCAAGSTSTATPLRVDYWGQGTLVTVSS

SEQ ID NO: 16 - protein, 122 aa, Artificial Sequence
  source: /note=“Description of Artificial Sequence: Synthetic polypeptide”
EVQLVESGGGLVQPGGSLRLSCAASGFKITHYTMGWFRQAPGKEREFVSRITWGGDNTFYSNSVKGRFTISRDNSKNTLYLQMNSLRAEDTAVYYCAAGSTSTATPLRVDYWGQGTLVTVSS

SEQ ID NO: 17 - protein, 122 aa, Artificial Sequence
  source: /note=“Description of Artificial Sequence: Synthetic polypeptide”
EVQLVESGGGLVQPGGSLRLSCAASGFKITHYTTGWFRQAPGKEREFVSRITWGGDNTFYSNSVKGRFTISRDNSKNTLYLQMNSLRAEDTAVYYCAAGSTSTATPLRVDYWGQGTLVTVSS

SEQ ID NO: 18 - protein, 226 aa, Artificial Sequence
  source: /note=“Description of Artificial Sequence: Synthetic polypeptide”
KTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGC

SEQ ID NO: 19 - protein, 9 aa, Artificial Sequence
  source: /note=“Description of Artificial Sequence: Synthetic peptide”
KTHTCPPCP

SEQ ID NO: 20 - protein, 122 aa, Artificial Sequence
  source: /note=“Description of Artificial Sequence: Synthetic polypeptide”
DVQLVESGGGLVQPGGSLRLSCAASGFKITHYTTGWFRQAPGKEREFVCRITWGGDNTFYSNSVKGRFTCSRDNSKNTVYLQMNSLRAEDTAVYYCAAGSTSTATPLRVDYWGQGTLVTVSS

SEQ ID NO: 21 - protein, 122 aa, Artificial Sequence
  source: /note=“Description of Artificial Sequence: Synthetic polypeptide”
EVQLVESGGGLVQPGGSLRLSCAASGFKITHYTTGWFRQAPGKGLEFVCRITWGGDNTFYSNSVKGRFTCSRDNSKNTVYLQMNSLRAEDTAVYYCAAGSTSTATPLRVDYWGQGTLVTVSS

SEQ ID NO: 22 - protein, 122 aa, Artificial Sequence
  source: /note=“Description of Artificial Sequence: Synthetic polypeptide”
EVQLVESGGGLVQPGGSLRLSCAASGFKITHYTCGWFRQAPGKGLEFVSRITWGGDNTFYSNSVKGRFTISRDNSKNTCYLQMNSLRAEDTAVYYCAAGSTSTATPLRVDYWGQGTLVTVSS

SEQ ID NO: 23 - protein, 122 aa, Artificial Sequence
  source: /note=“Description of Artificial Sequence: Synthetic polypeptide”
DVQLVESGGGLVQPGGSLRLSCAASGFKITHYTCGWFRQAPGKEREFVSRITWGGDNTFYSNSVKGRFTISRDNSKNTCYLQMNSLRAEDTAVYYCAAGSTSTATPLRVDYWGQGTLVTVSS

SEQ ID NO: 24 - protein, 122 aa, Artificial Sequence
  source: /note=“Description of Artificial Sequence: Synthetic polypeptide”
DVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTCTATPLRVDYWGQGTLVTVSS

SEQ ID NO: 25 - protein, 122 aa, Artificial Sequence
  source: /note=“Description of Artificial Sequence: Synthetic polypeptide”
DVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSCATPLRVDYWGQGTLVTVSS

SEQ ID NO: 26 - protein, 122 aa, Artificial Sequence
  source: /note=“Description of Artificial Sequence: Synthetic polypeptide”
DVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSS

SEQ ID NO: 27 - protein, 122 aa, Artificial Sequence
  source: /note=“Description of Artificial Sequence: Synthetic polypeptide”
DVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTACPLRVDYWGQGTLVTVSS

SEQ ID NO: 28 - protein, 122 aa, Artificial Sequence
  source: /note=“Description of Artificial Sequence: Synthetic polypeptide”
EVQLVESGGGLVQPGGSLRLSCAASGFKITHYCTGWFRQAPGKGLEFVCRITWGGDNTFYSNSVKGRFTCSRDNSKNTVYLQMNSLRAEDTAVYYCAAGSTSCATPLRVDYWGQGTLVTVSS

SEQ ID NO: 29 - protein, 442 aa, Artificial Sequence
  source: /note=“Description of Artificial Sequence: Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 442 aa, Artificial Sequence
  source: /note=“Description of Artificial Sequence: Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 438 aa, Artificial Sequence
  source: /note=“Description of Artificial Sequence: Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 32 - protein, 439 aa, Artificial Sequence
  source: /note=“Description of Artificial Sequence: Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 233 aa, Artificial Sequence
  source: /note=“Description of Artificial Sequence: Synthetic polypeptide”
AEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 599 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSGGGGSQVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 235 aa, Artificial Sequence
  source: /note=“Description of Artificial Sequence: Synthetic polypeptide”
MDMRVPAQLLGLLLWFPGSRCDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 360 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSGGGGSSYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 592 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 238 aa, Artificial Sequence
  source: /note=“Description of Artificial Sequence: Synthetic polypeptide”
MDMRVPAQLLGLLLLWFPGSRCEVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 39 - protein, 600 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSGGGGSQVQLVESGGGVVQPGRSLRVSCAASGFTFSSYDMHWVRQAPGKGLEWVAVIWFDGSNEFYADSVKGRFTISRDNSKNTLFLQMNSLRAEDTAVYYCARDLGASVTTSNAENFHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 236 aa, Artificial Sequence
  source: /note=“Description of Artificial Sequence: Synthetic polypeptide”
MDMRVPAQLLGLLLLWFPGSRCSYELTQPPSVSVSPGQTARITCSGDALPKRYVYWYQQKSGQAPVLVIYEDSKRPSGIPETFSGSSSGTMATLTISGAQVEDEADYYCYSTDSNGHHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 360 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSGGGGSSYELTQPPSVSVSPGQTARITCSGDALPKRYVYWYQQKSGQAPVLVIYEDSKRPSGIPETFSGSSSGTMATLTISGAQVEDEADYYCYSTDSNGHHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 476 aa, Artificial Sequence
  source: /note=“Description of Artificial Sequence: Synthetic polypeptide”
MDMRVPAQLLGLLLLWFPGSRCQVQLVESGGGVVQPGRSLRVSCAASGFTFSSYDMHWVRQAPGKGLEWVAVIWFDGSNEFYADSVKGRFTISRDNSKNTLFLQMNSLRAEDTAVYYCARDLGASVTTSNAENFHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 606 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSQVQLVESGGGVVQPGRSLRVSCAASGFTFSSYDMHWVRQAPGKGLEWVAVIWFDGSNEFYADSVKGRFTISRDNSKNTLFLQMNSLRAEDTAVYYCARDLGASVTTSNAENFHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 44 - protein, 236 aa, Artificial Sequence; identical to SEQ ID NO: 40
  source: /note=“Description of Artificial Sequence: Synthetic polypeptide”

SEQ ID NO: 45 - protein, 592 aa, Artificial Sequence
  source: /note=“Description of Artificial Sequence: Synthetic polypeptide”
MGWSLILLFLVAVATRVLSDVQLVESGGGLVQPGGSLRLSCAASGFKITHYCTGWFRQAPGKEREFVSRITWGGDNTFYSNSVKGRFTISRDNSKNTVYLQMNSLRAEDTAVYYCAAGSTSTCTPLRVDYWGQGTLVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSYELTQPPSVSVSPGQTARITCSGDALPKRYVYWYQQKSGQAPVLVIYEDSKRPSGIPETFSGSSSGTMATLTISGAQVEDEADYYCYSTDSNGHHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 250 aa, Artificial Sequence
  source: /note=“Description of Artificial Sequence: Synthetic polypeptide”
MDMRVPAQLLGLLLLWFPGSRCQVQLVESGGGVVQPGRSLRVSCAASGFTFSSYDMHWVRQAPGKGLEWVAVIWFDGSNEFYADSVKGRFTISRDNSKNTLFLQMNSLRAEDTAVYYCARDLGASVTTSNAENFHHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 47 - protein, 13 aa, Unknown
  source: /note=“Description of Unknown: Wild type FC5 CDR sequence”
AASGFKITHYTMG

SEQ ID NO: 48 - protein, 13 aa, Artificial Sequence
  source: /note=“Description of Artificial Sequence: Synthetic peptide”
AASGFKITHYTCG

SEQ ID NO: 49 - protein, 13 aa, Artificial Sequence
  source: /note=“Description of Artificial Sequence: Synthetic peptide”
AASGFKITHYCTG

SEQ ID NO: 50 - protein, 17 aa, Unknown
  source: /note=“Description of Unknown: Wild type FC5 CDR sequence”
RITWGGDNTFYSNSVKG

SEQ ID NO: 51 - protein, 15 aa, Unknown
  source: /note=“Description of Unknown: Wild type FC5 CDR sequence”
AAGSTSTATPLRVDY

SEQ ID NO: 52 - protein, 15 aa, Artificial Sequence
  source: /note=“Description of Artificial Sequence: Synthetic peptide”
AAGSTCTATPLRVDY

SEQ ID NO: 53 - protein, 15 aa, Artificial Sequence
  source: /note=“Description of Artificial Sequence: Synthetic peptide”
AAGSTSCATPLRVDY

SEQ ID NO: 54 - protein, 15 aa, Artificial Sequence
  source: /note=“Description of Artificial Sequence: Synthetic peptide”
AAGSTSTCTPLRVDY

SEQ ID NO: 55 - protein, 15 aa, Artificial Sequence
  source: /note=“Description of Artificial Sequence: Synthetic peptide”
AAGSTSTACPLRVDY

SEQ ID NO: 56 - protein, 7 aa, Artificial Sequence
  source: /note=“Description of Artificial Sequence: Synthetic peptide”
AEPKSCD

SEQ ID NO: 57 - protein, 13 aa, Artificial Sequence
  source: /note=“Description of Artificial Sequence: Synthetic peptide”
AASGFKITHYTTG

SEQ ID NO: 58 - protein, 13 aa, Artificial Sequence
  source: /note=“Description of Artificial Sequence: Synthetic peptide”
AASGFKITHYCMG

SEQ ID NO: 59 - protein, 7 aa, Artificial Sequence
  source: /note=“Description of Artificial Sequence: Synthetic peptide”
AEPKSSD

SEQ ID NO: 60 - protein, 15 aa, Artificial Sequence
  source: /note=“Description of Artificial Sequence: Synthetic peptide”
GGGGSGGGGSGGGGS

SEQ ID NO: 61 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 215 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 64 - protein, 215 aa, Artificial Sequence
  source: /note=“Description of Artificial Sequence: Synthetic polypeptide”
APPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 65 - protein, 216 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 66 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 67 - protein, 216 aa, Artificial Sequence
  source: /note=“Description of Artificial Sequence: Synthetic polypeptide”
APEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 69 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQKSLSLSPG

SEQ ID NO: 71 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 72 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVSTLPPSRDELTKNQVSLMCLVYGFYPSDIAVEWESNGQPENNYKTTPPVLDSDGSFFLYSVLTVDKSRWQQGNVFSCSVMHEALHNHYTQKSLSLSPG

SEQ ID NO: 73 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRGDLTKNQVQLTCLVKGFYPSDIAVEWESNGQPENNYKTTPPVLDSDGSFFLASKLTVDKSRWQQGNVFSCSVMHEALHNHYTQKSLSLSPG

SEQ ID NO: 74 - protein, 16 aa, Artificial Sequence
  source: /note=“Description of Artificial Sequence: Synthetic peptide”
AEPKSCDKTHTCPPCP

SEQ ID NO: 75 - protein, 16 aa, Artificial Sequence
  source: /note=“Description of Artificial Sequence: Synthetic peptide”
AEPKSSDKTHTCPPCP

SEQ ID NO: 76 - protein, 15 aa, Artificial Sequence
  source: /note=“Description of Artificial Sequence: Synthetic peptide”
GGGGSDKTHTCPPCP

SEQ ID NO: 77 - protein, 4 aa, Artificial Sequence
  source: /note=“Description of Artificial Sequence: Synthetic peptide”
GGGS

SEQ ID NO: 78 - protein, 4 aa, Artificial Sequence
  source: /note=“Description of Artificial Sequence: Synthetic peptide”
SGGG

SEQ ID NO: 79 - protein, 5 aa, Artificial Sequence
  source: /note=“Description of Artificial Sequence: Synthetic peptide”
SGGGG

SEQ ID NO: 80 - protein, 6 aa, Artificial Sequence
  source: /note=“Description of Artificial Sequence: Synthetic peptide”
GGGGGS

SEQ ID NO: 81 - protein, 6 aa, Artificial Sequence
  source: /note=“Description of Artificial Sequence: Synthetic peptide”
SGGGGG

SEQ ID NO: 82 - protein, 7 aa, Artificial Sequence
  source: /note=“Description of Artificial Sequence: Synthetic peptide”
GGGGGGS

SEQ ID NO: 83 - protein, 7 aa, Artificial Sequence
  source: /note=“Description of Artificial Sequence: Synthetic peptide”
SGGGGGG

SEQ ID NO: 84 - protein, 25 aa, Artificial Sequence
  source: /note=“Description of Artificial Sequence: Synthetic peptide”
  MOD_RES (4)..(5): Any amino acid
  MOD_RES (9)..(10): Any amino acid
  MOD_RES (14)..(15): Any amino acid
  MOD_RES (19)..(20): Any amino acid
  SITE (1)..(20): /note=“This region may encompass 0-4 ′Gly Gly Gly Xaa Xaa′ repeating units”
  source: /note=“See specification as filed for detailed description of substitutions and preferred embodiments”
GGGXXGGGXXGGGXXGGGXXGGGGS

SEQ ID NO: 85 - protein, 25 aa, Artificial Sequence
  source: /note=“Description of Artificial Sequence: Synthetic peptide”
  SITE (6)..(25): /note=“This region may encompass 0-4 ′Xaa Gly Gly Gly Ser′ repeating units”
  MOD_RES (6)..(6): Any amino acid
  MOD_RES (11)..(11): Any amino acid
  MOD_RES (16)..(16): Any amino acid
  MOD_RES (21)..(21): Any amino acid
  source: /note=“See specification as filed for detailed description of substitutions and preferred embodiments”
GGGGSXGGGSXGGGSXGGGSXGGGS

SEQ ID NO: 86 - protein, 25 aa, Artificial Sequence
  source: /note=“Description of Artificial Sequence: Synthetic peptide”
  SITE (1)..(20): /note=“This region may encompass 0-4 ′Gly Gly Gly Pro Ser′ repeating units”
GGGPSGGGPSGGGPSGGGPSGGGGS

SEQ ID NO: 87 - protein, 25 aa, Artificial Sequence
  source: /note=“Description of Artificial Sequence: Synthetic peptide”
  SITE (1)..(20): /note=“This region may encompass 0-4 ′Gly Gly Gly Gly Gln′ repeating units”
GGGGQGGGGQGGGGQGGGGQGGGGS

SEQ ID NO: 88 - protein, 25 aa, Artificial Sequence
  source: /note=“Description of Artificial Sequence: Synthetic peptide”
  SITE (1)..(20): /note=“This region may encompass 0-4 ′Gly Gly Gly Gly Ala′ repeating units”
GGGGAGGGGAGGGGAGGGGAGGGGS

SEQ ID NO: 89 - protein, 25 aa, Artificial Sequence
  source: /note=“Description of Artificial Sequence: Synthetic peptide”
  SITE (6)..(25): /note=“This region may encompass 0-4 ′Pro Gly Gly Gly Ser′ repeating units”
GGGGSPGGGSPGGGSPGGGSPGGGS

SEQ ID NO: 90 - protein, 15 aa, Artificial Sequence
  source: /note=“Description of Artificial Sequence: Synthetic peptide”
GGGGAGGGGAGGGGS

SEQ ID NO: 91 - protein, 15 aa, Artificial Sequence
  source: /note=“Description of Artificial Sequence: Synthetic peptide”
GGGGQGGGGQGGGGS

SEQ ID NO: 92 - protein, 15 aa, Artificial Sequence
  source: /note=“Description of Artificial Sequence: Synthetic peptide”
GGGPSGGGPSGGGGS

SEQ ID NO: 93 - protein, 15 aa, Artificial Sequence
  source: /note=“Description of Artificial Sequence: Synthetic peptide”
GGGGSPGGGSPGGGS

SEQ ID NO: 94 - protein, 585 aa, Homo sapiens [sequence omitted]

SEQ ID NO: 95 - protein, 12 aa, Artificial Sequence
  source: /note=“Description of Artificial Sequence: Synthetic peptide”
ERKSCVECPPCP

SEQ ID NO: 96 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVDGFYPSDIAVEWESNGQPENNYKTTPPVLMSDGSFFLASKLTVDKSRWQQGNVFSCSVMHEALHNHYTQKSLSLSPG

SEQ ID NO: 97 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSRDELTKNQVSLVCLVKGFYPSDIAVEWESNGQPENNYKTTPPVLDSDGSFFLYSVLTVDKSRWQQGNVFSCSVMHEALHNHYTQKSLSLSPG

SEQ ID NO: 98 - protein, 140 aa, Artificial Sequence
  source: /note=“Description of Artificial Sequence: Synthetic polypeptide”
TVLHQDWLNGKEYKCKVSNKALPAPIEKTISKAKGQPREPQVYTLPPSRDELTKNQVKLTCLVKGFYPSDIAVEWESNGQPENNYKTTPPVLDSDGSFFLYSLLTVDKSRWQQGNVFSCSVMHEALHNHYTQKSLSLSPG

SEQ ID NO: 99 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVSGFYPSDIAVEWESNGQPENNYKTTPPVLDSDGSFKLYSKLTVDKSRWQQGNVFSCSVMHEALHNHYTQKSLSLSPG

SEQ ID NO: 100 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 101 - protein, 216 aa, Artificial Sequence
  source: /note=“Description of Artificial Sequence: Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 102 - protein, 13 aa, Artificial Sequence
  source: /note=“Description of Artificial Sequence: Synthetic peptide”
VERKCCVECPPCP

SEQ ID NO: 103 - protein, 13 aa, Artificial Sequence
  source: /note=“Description of Artificial Sequence: Synthetic peptide”
VESKYGPPCPSCP

SEQ ID NO: 104 - protein, 17 aa, Artificial Sequence
  source: /note=“Description of Artificial Sequence: Synthetic peptide”
ELKTPLGDTTHTCPRCP

SEQ ID NO: 105 - protein, 15 aa, Artificial Sequence
  source: /note=“Description of Artificial Sequence: Synthetic peptide”
EPKSCDTPPPCPRCP

SEQ ID NO: 106 - protein, 42 aa, Artificial Sequence
  source: /note=“Description of Artificial Sequence: Synthetic polypeptide”
GAPGSPAGSPTSTEEGTSESATPESGPGSEPATSGSETPASS

SEQ ID NO: 107 - protein, 42 aa, Artificial Sequence
  source: /note=“Description of Artificial Sequence: Synthetic polypeptide”
TGGGSPAGSPTSTEEGTSESATPESGPGSEPATSGSETPASS

SEQ ID NO: 108 - protein, 42 aa, Artificial Sequence
  source: /note=“Description of Artificial Sequence: Synthetic polypeptide”
GTSESATPESGPGSEPATSGSETPGTSESATPESGPGSEPAT

SEQ ID NO: 109 - protein, 78 aa, Artificial Sequence
  source: /note=“Description of Artificial Sequence: Synthetic polypeptide”
GAPTSESATPESGPGSEPATSGSETPGTSESATPESGPGSEPATSGSETPGTSESATPESGPGTSTEPSEGSAPGASS

SEQ ID NO: 110 - protein, 72 aa, Artificial Sequence
  source: /note=“Description of Artificial Sequence: Synthetic polypeptide”
GTSESATPESGPGSEPATSGSETPGTSESATPESGPGSEPATSGSETPGTSESATPESGPGTSTEPSEGSAP

SEQ ID NO: 111 - protein, 72 aa, Artificial Sequence
  source: /note=“Description of Artificial Sequence: Synthetic polypeptide”
SPAGSPTSTEEGTSESATPESGPGSEPATSGSETPGTSESATPESGPGTSTEPSEGSAPGTSTEPSEGSAPG

SEQ ID NO: 112 - protein, 143 aa, Artificial Sequence
  source: /note=“Description of Artificial Sequence: Synthetic polypeptide”
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESAPESGPGSEPATSGSETPGTSTEPSEGSAP

SEQ ID NO: 113 - protein, 144 aa, Artificial Sequence
  source: /note=“Description of Artificial Sequence: Synthetic polypeptide”
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEE

SEQ ID NO: 114 - protein, 144 aa, Artificial Sequence
  source: /note=“Description of Artificial Sequence: Synthetic polypeptide”
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAP

SEQ ID NO: 115 - protein, 144 aa, Artificial Sequence
  source: /note=“Description of Artificial Sequence: Synthetic polypeptide”
GTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSP

SEQ ID NO: 116 - protein, 288 aa, Artificial Sequence
  source: /note=“Description of Artificial Sequence: Synthetic polypeptide”
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 117 - protein, 288 aa, Artificial Sequence
  source: /note=“Description of Artificial Sequence: Synthetic polypeptide”
PGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGS

SEQ ID NO: 118 - protein, 576 aa, Artificial Sequence
  source: /note=“Description of Artificial Sequence: Synthetic polypeptide”
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 119 - protein, 576 aa, Artificial Sequence
  source: /note=“Description of Artificial Sequence: Synthetic polypeptide”
PGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGS

SEQ ID NO: 120 - protein, 864 aa, Artificial Sequence
  source: /note=“Description of Artificial Sequence: Synthetic polypeptide”
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 121 - protein, 864 aa, Artificial Sequence
  source: /note=“Description of Artificial Sequence: Synthetic polypeptide”
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 122 - protein, 5 aa, Artificial Sequence
  source: /note=“Description of Artificial Sequence: Synthetic peptide”
AYEMK

SEQ ID NO: 123 - protein, 17 aa, Artificial Sequence
  source: /note=“Description of Artificial Sequence: Synthetic peptide”
VIGPSGGFTFYADSVKG

SEQ ID NO: 124 - protein, 9 aa, Artificial Sequence
  source: /note=“Description of Artificial Sequence: Synthetic peptide”
EGDNDAFDI

SEQ ID NO: 125 - protein, 11 aa, Artificial Sequence
  source: /note=“Description of Artificial Sequence: Synthetic peptide”
RASQSVSSYLA

SEQ ID NO: 126 - protein, 7 aa, Artificial Sequence
  source: /note=“Description of Artificial Sequence: Synthetic peptide”
DASNRAT

SEQ ID NO: 127 - protein, 10 aa, Artificial Sequence
  source: /note=“Description of Artificial Sequence: Synthetic peptide”
QQRSNWPMYT

SEQ ID NO: 128 - protein, 10 aa, Artificial Sequence
  source: /note=“Description of Artificial Sequence: Synthetic peptide”
GFTFSRYAMS

SEQ ID NO: 129 - protein, 17 aa, Artificial Sequence
  source: /note=“Description of Artificial Sequence: Synthetic peptide”
EISSGGSYPYYPDTVTG

SEQ ID NO: 130 - protein, 16 aa, Artificial Sequence
  source: /note=“Description of Artificial Sequence: Synthetic peptide”
VLYYDYDGDRIEVMDY

SEQ ID NO: 131 - protein, 16 aa, Artificial Sequence
  source: /note=“Description of Artificial Sequence: Synthetic peptide”
RSSQSLVSSKGNTYLH

SEQ ID NO: 132 - protein, 7 aa, Artificial Sequence
  source: /note=“Description of Artificial Sequence: Synthetic peptide”
KVSNRFS

SEQ ID NO: 133 - protein, 9 aa, Artificial Sequence
  source: /note=“Description of Artificial Sequence: Synthetic peptide”
SQSTHFPRT

SEQ ID NO: 134 - protein, 5 aa, Artificial Sequence
  source: /note=“Description of Artificial Sequence: Synthetic peptide”
SYGMH

SEQ ID NO: 135 - protein, 17 aa, Artificial Sequence
  source: /note=“Description of Artificial Sequence: Synthetic peptide”
VIWFDGTKKYYTDSVKG

SEQ ID NO: 136 - protein, 15 aa, Artificial Sequence
  source: /note=“Description of Artificial Sequence: Synthetic peptide”
DRGIGARRGPYYMDV

SEQ ID NO: 137 - protein, 11 aa, Artificial Sequence
  source: /note=“Description of Artificial Sequence: Synthetic peptide”
RASQSISSYLN

SEQ ID NO: 138 - protein, 7 aa, Artificial Sequence
  source: /note=“Description of Artificial Sequence: Synthetic peptide”
AASSLQS

SEQ ID NO: 139 - protein, 9 aa, Artificial Sequence
  source: /note=“Description of Artificial Sequence: Synthetic peptide”
QQSYSTPLT

SEQ ID NO: 140 - protein, 10 aa, Artificial Sequence
  source: /note=“Description of Artificial Sequence: Synthetic peptide”
GFTFSSYDMH

SEQ ID NO: 141 - protein, 17 aa, Artificial Sequence
  source: /note=“Description of Artificial Sequence: Synthetic peptide”
VIWFDGSNEFYADSVKG

SEQ ID NO: 142 - protein, 16 aa, Artificial Sequence
  source: /note=“Description of Artificial Sequence: Synthetic peptide”
DLGASVTTSNAENFHH

SEQ ID NO: 143 - protein, 11 aa, Artificial Sequence
  source: /note=“Description of Artificial Sequence: Synthetic peptide”
SGDALPKRYVY

SEQ ID NO: 144 - protein, 7 aa, Artificial Sequence
  source: /note=“Description of Artificial Sequence: Synthetic peptide”
EDSKRPS

SEQ ID NO: 145 - protein, 11 aa, Artificial Sequence
  source: /note=“Description of Artificial Sequence: Synthetic peptide”
YSTDSNGHHWV

SEQ ID NO: 146-148 - skipped (3 entries)

SEQ ID NO: 149 - protein, 10 aa, Artificial Sequence
  source: /note=“Description of Artificial Sequence: Synthetic peptide”
GFTFPNYVMT

SEQ ID NO: 150 - protein, 18 aa, Artificial Sequence
  source: /note=“Description of Artificial Sequence: Synthetic peptide”
SGISGSGGSTDYADSVKG

SEQ ID NO: 151 - protein, 5 aa, Artificial Sequence
  source: /note=“Description of Artificial Sequence: Synthetic peptide”
GSGGI

SEQ ID NO: 152 - protein, 14 aa, Artificial Sequence
  source: /note=“Description of Artificial Sequence: Synthetic peptide”
TGTSSDVGGYNYVS

SEQ ID NO: 153 - protein, 7 aa, Artificial Sequence
  source: /note=“Description of Artificial Sequence: Synthetic peptide”
DVTKRPS

SEQ ID NO: 154 - protein, 10 aa, Artificial Sequence
  source: /note=“Description of Artificial Sequence: Synthetic peptide”
HSYVGSYTLV

SEQ ID NO: 155 - protein, 5 aa, Artificial Sequence
  source: /note=“Description of Artificial Sequence: Synthetic peptide”
KYGMS

SEQ ID NO: 156 - protein, 17 aa, Artificial Sequence
  source: /note=“Description of Artificial Sequence: Synthetic peptide”
TISSSGSRTYYPDSVKG

SEQ ID NO: 157 - protein, 8 aa, Artificial Sequence
  source: /note=“Description of Artificial Sequence: Synthetic peptide”
SWDGAMDY

SEQ ID NO: 158 - protein, 16 aa, Artificial Sequence
  source: /note=“Description of Artificial Sequence: Synthetic peptide”
KSSQSIVHSNGNTYLE

SEQ ID NO: 159 - protein, 7 aa, Artificial Sequence
  source: /note=“Description of Artificial Sequence: Synthetic peptide”
KVSNRFS

SEQ ID NO: 160 - protein, 9 aa, Artificial Sequence
  source: /note=“Description of Artificial Sequence: Synthetic peptide”
FQGSLVPWA

SEQ ID NO: 161 - protein, 5 aa, Artificial Sequence
  source: /note=“Description of Artificial Sequence: Synthetic peptide”
KAWMS

SEQ ID NO: 162 - protein, 10 aa, Artificial Sequence
  source: /note=“Description of Artificial Sequence: Synthetic peptide”
GFDFEKAWMS

SEQ ID NO: 163 - protein, 19 aa, Artificial Sequence
  source: /note=“Description of Artificial Sequence: Synthetic peptide”
RIKSTADGGTTSYAAPVEG

SEQ ID NO: 164 - protein, 2 aa, Artificial Sequence
  source: /note=“Description of Artificial Sequence: Synthetic peptide”
AH

SEQ ID NO: 165 - protein, 11 aa, Artificial Sequence
  source: /note=“Description of Artificial Sequence: Synthetic peptide”
SGEALPMQFAH

SEQ ID NO: 166 - protein, 7 aa, Artificial Sequence
  source: /note=“Description of Artificial Sequence: Synthetic peptide”
KDSERPS

SEQ ID NO: 167 - protein, 11 aa, Artificial Sequence
  source: /note=“Description of Artificial Sequence: Synthetic peptide”
QSPDSTNTYEV

SEQ ID NO: 168 - protein, 10 aa, Artificial Sequence
  source: /note=“Description of Artificial Sequence: Synthetic peptide”
GFTFSTYYMS

SEQ ID NO: 169 - protein, 17 aa, Artificial Sequence
  source: /note=“Description of Artificial Sequence: Synthetic peptide”
NIKQDGSEKYYVDSVKG

SEQ ID NO: 170 - protein, 4 aa, Artificial Sequence
  source: /note=“Description of Artificial Sequence: Synthetic peptide”
PPGW

SEQ ID NO: 171 - protein, 16 aa, Artificial Sequence
  source: /note=“Description of Artificial Sequence: Synthetic peptide”
KSSQSLLHSDGKTYLY

SEQ ID NO: 172 - protein, 7 aa, Artificial Sequence
  source: /note=“Description of Artificial Sequence: Synthetic peptide”
EVSNRFS

SEQ ID NO: 173 - protein, 9 aa, Artificial Sequence
  source: /note=“Description of Artificial Sequence: Synthetic peptide”
MQSIQLPVT

SEQ ID NO: 174 - protein, 6 aa, Artificial Sequence
  source: /note=“Description of Artificial Sequence: Synthetic peptide”
GGSVSD

SEQ ID NO: 175 - protein, 18 aa, Artificial Sequence
  source: /note=“Description of Artificial Sequence: Synthetic peptide”
RTYTNGKTTYTYNPSLES

SEQ ID NO: 176 - protein, 14 aa, Artificial Sequence
  source: /note=“Description of Artificial Sequence: Synthetic peptide”
WGAVTGDYYYGMDV

SEQ ID NO: 177 - protein, 11 aa, Artificial Sequence
  source: /note=“Description of Artificial Sequence: Synthetic peptide”
RASQNIDKYLN

SEQ ID NO: 178 - protein, 7 aa, Artificial Sequence
  source: /note=“Description of Artificial Sequence: Synthetic peptide”
AASSLHS

SEQ ID NO: 179 - protein, 9 aa, Artificial Sequence
  source: /note=“Description of Artificial Sequence: Synthetic peptide”
QQSYSSFRT

SEQ ID NO: 180 - protein, 16 aa, Artificial Sequence
  source: /note=“Description of Artificial Sequence: Synthetic peptide”
RSPRSLLHTNGYTYLD

SEQ ID NO: 181 - protein, 7 aa, Artificial Sequence
  source: /note=“Description of Artificial Sequence: Synthetic peptide”
LASNRAS

SEQ ID NO: 182 - protein, 9 aa, Artificial Sequence
  source: /note=“Description of Artificial Sequence: Synthetic peptide”
MQGLQPSWT

SEQ ID NO: 183 - protein, 118 aa, Artificial Sequence [sequence omitted]

SEQ ID NO: 184 - protein, 108 aa, Artificial Sequence
  source: /note=“Description of Artificial Sequence: Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIK

SEQ ID NO: 185 - protein, 125 aa, Artificial Sequence
  source: /note=“Description of Artificial Sequence: Synthetic polypeptide”
EVQLVESGGGLVQPGGSLRLSCAASGFTFSRYAMSWVRQAPGKGLEWVAEISSGGSYPYYPDTVTGRFTISRDNAKNSLYLQMNSLRAEDTAVYYCARVLYYDYDGDRIEVMDYWGQGTLVTVSS

SEQ ID NO: 186 - protein, 112 aa, Artificial Sequence
  source: /note=“Description of Artificial Sequence: Synthetic polypeptide”
DVVMTQSPLSLPVTPGEPASISCRSSQSLVSSKGNTYLHWYLQKPGQSPQLLIYKVSNRFSGVPDRFSGSGSGTDFTLKISRVEAEDVGVYYCSQSTHFPRTFGGGTKVEIK

SEQ ID NO: 187 - protein, 124 aa, Artificial Sequence
  source: /note=“Description of Artificial Sequence: Synthetic polypeptide”
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 188 - protein, 108 aa, Artificial Sequence
  source: /note=“Description of Artificial Sequence: Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 189 - protein, 125 aa, Artificial Sequence
  source: /note=“Description of Artificial Sequence: Synthetic polypeptide”
QVQLVESGGGVVQPGRSLRVSCAASGFTFSSYDMHWVRQAPGKGLEWVAVIWFDGSNEFYADSVKGRFTISRDNSKNTLFLQMNSLRAEDTAVYYCARDLGASVTTSNAENFHHWGQGTLVTVSS

SEQ ID NO: 190 - protein, 108 aa, Artificial Sequence
  source: /note=“Description of Artificial Sequence: Synthetic polypeptide”
SYELTQPPSVSVSPGQTARITCSGDALPKRYVYWYQQKSGQAPVLVIYEDSKRPSGIPETFSGSSSGTMATLTISGAQVEDEADYYCYSTDSNGHHWVFGGGTKLTVL

SEQ ID NO: 191 - protein, 114 aa, Artificial Sequence
  source: /note=“Description of Artificial Sequence: Synthetic polypeptide”
EVQLVESGGGLVQPGGSLRLSCAASGFTFPNYVMTWVRQAPGKGLEWVSGISGSGGSTDYADSVKGRFTISRDNSKNTLYLQMNSLRVEDTALYYCAKGSGGIRGQGTMVTVSS

SEQ ID NO: 192 - protein, 110 aa, Artificial Sequence
  source: /note=“Description of Artificial Sequence: Synthetic polypeptide”
QSALTQPRSVSGSPGQSVTISCTGTSSDVGGYNYVSWHQQHPGKAPKLLIYDVTKRPSGVPDRFSGSKSGNTAALTISGLQAEDEADYYCHSYVGSYTLVFGGGTKLTVL

SEQ ID NO: 193 - protein, 117 aa, Artificial Sequence
  source: /note=“Description of Artificial Sequence: Synthetic polypeptide”
EVHLVESGGALVKPGGSLRLSCAASGFSFSKYGMSWVRQAPGKGLEWVATISSSGSRTYYPDSVKGRFTISRDNAKNTLYLQMNSLRAEDTAMYYCSISWDGAMDYWGQGTTVTVSS

SEQ ID NO: 194 - protein, 112 aa, Artificial Sequence
  source: /note=“Description of Artificial Sequence: Synthetic polypeptide”
DVVMTQSPLSLPVTLGQPASISCKSSQSIVHSNGNTYLEWYLQKPGQSPQLLVYKVSNRFSGVPDRFSGSGSGTDFTLKISRVEAEDVGTYYCFQGSLVPWAFGGGTKVEIK

SEQ ID NO: 195 - protein, 113 aa, Artificial Sequence
  source: /note=“Description of Artificial Sequence: Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSS

SEQ ID NO: 196 - protein, 108 aa, Artificial Sequence
  source: /note=“Description of Artificial Sequence: Synthetic polypeptide”
SYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVL

SEQ ID NO: 197 - protein, 123 aa, Artificial Sequence
  source: /note=“Description of Artificial Sequence: Synthetic polypeptide”
QVQLVESGGGVVQPGRSLRLSCAASGFTFSNHAMHWVRQAPGKGLEWVAVISYDGENTYYADSIEGRFTISRDNFKNTLFLQMYSLTADDTAMYFCARGGRRGHFTSYYLDYWGQGTLVTVSS

SEQ ID NO: 198 - protein, 107 aa, Artificial Sequence
  source: /note=“Description of Artificial Sequence: Synthetic polypeptide”
DIQMTQSPSSLSASVGDRVTITCRASQNIDKYLNWYQQIPGKAPKLLIYAASSLHSGVPSRFSGSGSGTDFSLTISSLQPEDFAIYYCQQSYSSFRTFGQGTKLEIK

SEQ ID NO: 199 - protein, 124 aa, Artificial Sequence
  source: /note=“Description of Artificial Sequence: Synthetic polypeptide”
QVQLQESGPGLVKPSETLSLTYTVLGGSVSDYYWSCIRQPAGKGLEWIGRTYTNGKTTYTYNPSLESRLSLSIDTSMNQFSLKLTSVTAADTAVYYCARWGAVTGDYYYGMDVWGPGTLVTVSS

SEQ ID NO: 200 - protein, 112 aa, Artificial Sequence
  source: /note=“Description of Artificial Sequence: Synthetic polypeptide”
EIVLTQSPLSLSVTPGEPASISCRSPRSLLHTNGYTYLDWYLQRPGQSPQLLIFLASNRASGVPDRFSGSGSGTNFTLRISGVEADDVGVYYCMQGLQPSWTFGQGTKVEIK

SEQ ID NO: 201 - protein, 10 aa, Artificial Sequence
  source: /note=“Description of Artificial Sequence: Synthetic peptide”
GFTFSNHAMH

SEQ ID NO: 202 - protein, 17 aa, Artificial Sequence
  source: /note=“Description of Artificial Sequence: Synthetic peptide”
VISYDGENTYYADSIEG

SEQ ID NO: 203 - protein, 14 aa, Artificial Sequence
  source: /note=“Description of Artificial Sequence: Synthetic peptide”
GGRRGHFTSYYLDY

SEQ ID NO: 204 - protein, 12 aa, Artificial Sequence
  source: /note=“Description of Artificial Sequence: Synthetic peptide”
GAGAGAGAGAGA